(19) United States
(12) Patent Application Publication
(10) Pub. No.: US 2008/0118529 A1
(43) Pub. Date: May 22, 2008

(54) Adjuvation Through Cross -Beta Structure

(76) Inventors: Martijn Frans Ben Gerard Gebbink, Eemnes (NL); Barend Bouma, Houten (NL)

Correspondence Address: TRASK BRITT, P.O. BOX 2550, SALT LAKE CITY, UT, 84110, US

(21) Appl. No.: 11/661,537

(22) PCT Filed: Jul. 13, 2006
(86) PCT No.: PCT/NL06/00362

§ 371 Date: Apr. 24, 2007

(30) Foreign Application Priority Data
Jul. 13, 2005 (EP) 05076612.0

(51) Int. Cl. A61K 38/00; C07K 16/00; A61K 39/00; C12Q 1/00

(52) U.S. Cl. 424/204.1; 530/300; 530/350; 435/4; 514/8

(57) ABSTRACT

The invention relates to novel methods and means for providing proteinaceous substances, such as peptides, polypeptides, glycoproteins, lipoproteins and complex compounds comprising the former in combination with other substances, such as nucleic acids, membrane structures, carbohydrate structures, with cross-β structures, which enhance the immunogenicity of said proteinaceous substance. The resulting peptides, proteins, glycoproteins, etc. are preferably used in vaccines. The invention provides a method for producing an immunogenic composition comprising at least one peptide, polypeptide, protein, glycoprotein and/or lipoprotein, comprising providing said composition with at least one cross-β structure. The invention also discloses the use of cross-β structures in the preparation of a vaccine for the prophylaxis of an infectious disease. The invention further provides a method for improving immunogenicity of a composition comprising at least one peptide, polypeptide, protein, glycoprotein and/or lipoprotein, comprising contacting at least one of said peptide, polypeptide, protein, glycoprotein and/or lipoprotein with a cross-β inducing agent, thereby providing said composition with additional cross-β structures.

The invention relates to novel methods and means for providing proteinaceous substances, such as peptides, polypeptides, glycoproteins, lipoproteins and complex compounds comprising the former in combination with other substances, such as nucleic acids, membrane structures, carbohydrate structures, with cross-β structures, which enhance the immunogenicity of said proteinaceous substance. The resulting peptides, proteins, glycoproteins, etc. are used in vaccines.

Vaccines can be divided in two basic groups, i.e. prophylactic vaccines and therapeutic vaccines. Prophylactic vaccines have been made and/or suggested against essentially every known infectious agent (virus, bacterium, yeast, fungi, parasite, mycoplasm, etc.), which has some pathology in man, pets and/or livestock. Therapeutic vaccines have been made and/or suggested for infectious agents as well, but also for treatments of cancer and other aberrancies, as well as for inducing immune responses against other self antigens, as widely ranging as e.g. LHRH for immunocastration of boars, or for use in preventing graft versus host (GvH) and/or transplant rejections.

In vaccines in general there are two vital issues. Vaccines have to be efficacious and vaccines have to be safe. It often seems that the two requirements are mutually exclusive when trying to develop a vaccine. The most efficacious vaccines so far have been modified live infectious agents. These are modified in a manner that their virulence has been reduced (attenuation) to an acceptable level. The vaccine strain of the infectious agent typically does replicate in the host, but at a reduced level, so that the host can mount an adequate immune response, also providing the host with long term immunity against the infectious agent. The downside of attenuated vaccines is that the infectious agents may revert to a more virulent (and thus pathogenic) form.

This may happen in any infectious agent, but is a very serious problem in fast mutating viruses (such as in particular RNA viruses). Another problem with modified live vaccines is that infectious agents often have many different serotypes. It has proven to be difficult in many cases to provide vaccines which elicit an immune response in a host that protects against different serotypes of infectious agents.

Vaccines in which the infectious agent has been killed are safe, but often their efficacy is mediocre at best, even when the vaccine contains an adjuvant.

A type of vaccine that has received a lot of attention since the advent of modern biology is the subunit vaccine. In these vaccines only a few elements of the infectious agent are used to elicit an immune response. Typically a subunit vaccine comprises two or three proteins (glycoproteins) and/or peptides present in proteins of an infectious agent (from one or more serotypes) which have been produced by recombinant means and/or synthetic means. Although these vaccines in theory are the most promising safe and efficacious vaccines, in practice efficacy has proved to be a major hurdle. Molecular biology has provided more alternative methods to arrive at safe and efficacious vaccines that theoretically should also provide cross-protection against different serotypes of infectious agents. Carbohydrate structures derived from infectious agents have been suggested as specific immune response eliciting components of vaccines, as well as lipopolysaccharide structures, even nucleic acid complexes have been proposed. Although these component vaccines are generally safe, their efficacy and cross-protection over different serotypes has been generally lacking. Combinations of different kinds of components have been suggested (carbohydrates with peptides/proteins and lipopolysaccharide (LPS) with peptides/proteins optionally with carriers), but so far the safety vs. efficacy issue remains.

Another approach to provide cross protection is to make hybrid infectious agents which comprise antigenic components from two or more serotypes of an infectious agent. These can be and have been produced by modern molecular biology techniques. They can be produced as modified live vaccines, or as vaccines with inactivated or killed pathogens, but also as subunit vaccines. Cocktail vaccines comprising antigens from completely different infectious agents are also well known. In many countries children are routinely vaccinated with cocktail vaccines against e.g. diphteria, whooping cough, tetanus and polio. Recombinant vaccines comprising antigenic elements from different infectious agents have also been suggested. For instance for poultry a vaccine based on a chicken anemia virus has been suggested to be complemented with antigenic elements of Marek disease virus (MDV), but many more combinations have been suggested and produced.

Another important advantage of modern recombinant vaccines is that they have provided the opportunity to produce marker vaccines. Marker vaccines have been provided with an extra element that is not present in wild type infectious agent, or marker vaccines lack an element that is present in wild type infectious agent. The response of a host to both types of marker vaccines can be distinguished (typically by serological diagnosis) from the response against an infection with wild type.

The present invention provides methods and means which improve the immunogenicity of compositions intended to elicit an immune response.

In particular the invention provides compositions with enhanced immunogenicity for use as vaccines, be it prophylactic or therapeutic. The invention also provides vaccines with improved immunogenicity and improved safety.

In one embodiment the invention provides a method for producing an immunogenic composition comprising at least one peptide, polypeptide, protein, glycoprotein and/or lipoprotein, comprising providing said composition with at least one cross-β structure. A cross-β structure is defined as a part of a protein or peptide, or a part of an assembly of peptides and/or proteins, which comprises an ordered group of β-strands, typically a group of β-strands arranged in a β-sheet, in particular a group of stacked and layered β-sheets. A typical form of stacked β-sheets is in a fibril-like structure in which the β-sheets may be stacked in either the direction of the axis of the fibril or perpendicular to the direction of the axis of the fibril. The term structure can be used interchangeably with the term conformation. Of course the term peptide is intended to include oligopeptides as well as polypeptides, and the term protein includes proteins with and without post-translational modifications, such as glycosylation. It also includes lipoproteins and complexes comprising proteins, such as protein-nucleic acid complexes (RNA and/or DNA), membrane-protein complexes, etc.

To provide an immunogenic composition, particularly an immunogenic composition intended to elicit a specific immune response against a specific (group of) antigens with a cross-β structure, a protein or peptide as defined above comprising a cross-β structure can be simply added to said composition. Preferably said protein or peptide comprising said cross-β structure is an otherwise inert peptide or protein. Inert is defined as not eliciting an unwanted immune response or another unwanted biochemical reaction in a host, at least not to an unacceptable degree, preferably only to a negligible degree. The desired function should of course be present through the presence of cross-β structures. The protein or peptide comprising a cross-β structure may be added to any kind of vaccine, be it therapeutic or prophylactic, be it attenuated or killed whole infectious agent, be it subunit vaccine or carbohydrate or LPS vaccine or combinations thereof. A cross-β structure may be present in a single proteinaceous compound or may be a structure shared by several proteinaceous compounds. Cross-β structures can be induced through many different mechanisms. Many kinds of denaturing processes for proteins and/or polypeptides lead to the formation of cross-β structures. Such denaturing processes can therefore be applied to induce cross-β structures in many kinds of polypeptides and/or proteins. Examples are heating, chemical treatments with e.g. high salts, acid or alkaline materials, pressure and other physical treatments, etc. The addition of a cross-β structure may provide a composition with immunogenicity in a host, it may also enhance any immunogenicity already present in a composition. The cross-β structure providing protein/polypeptide/peptide may be added to a composition by itself, but it is also useful to use said cross-β structure providing proteinaceous substance as a carrier to which elements of the infectious agent(s) and/or antigen(s) are linked. This linkage can be provided through chemical linking (direct or indirect) or by expression of the relevant antigen(s) and the cross-β structure providing proteinaceous substance as a fusion protein. In both cases linkers between the two may be present. In both cases dimers, trimers and/or multimers of the antigen (or one or more epitopes of a relevant antigen) may be coupled to the cross-β structure providing proteinaceous compound. However, normal carriers comprising relevant epitopes or antigens coupled to them may also be used. The simple addition of a cross-β structure comprising proteinaceous substance will enhance the immunogenicity of such a complex. This is more or less generally true. An immunogenic composition according to the invention may typically comprise a number or all of the normal constituents of an immunogenic composition (in particular a vaccine), supplemented with a cross-β structure (conformation) comprising proteinaceous compound.

In a preferred embodiment the polypeptide/protein providing the cross-β structure is itself a vaccine component (i.e. derived from the infectious agent or antigen against which an immune response is desired).

Thus in a further embodiment the invention provides a method according to the invention, wherein said cross-β structure is induced in at least part of said at least one peptide, polypeptide, protein, glycoprotein and/or lipoprotein.

In this embodiment a part of the desired antigen and/or antigens or one or more epitopes from said antigen(s) is used as the cross-β structure providing compound. As stated before cross-β structures can be introduced in many ways. A preferred manner of introducing cross-β structures in an antigen is by one or more treatments of heating, freezing, oxidation, glycation pegylation, sulphatation, exposure to a chaotroph, preferably the chaotroph is urea or guanidinium-HCl, phosphorylation, partial proteolysis, chemical lysis, preferably with HCl or cyanogenbromide, sonication, dissolving in organic solutions, preferably 1,1,1,3,3,3-hexafluoro-2-propanol and trifluoroacetic acid, or a combination thereof.

An epitope in itself may be too small to comprise cross-β structures. As stated before, several epitopes together may form a cross-β structure and/or the epitope can be put (synthetically, chemically or recombinantly) in an environment comprising cross-β structures. Thus, in a subunit vaccine comprising e.g. two proteinaceous antigens from an infectious agent, a part of one or the other or both antigens can be provided with cross-β structures and then put back together with the rest of the antigenic material to provide or at least improve the immunogenicity of a composition comprising these antigens. Again, normal constituents of immunogenic compositions (in particular vaccines) such as carriers, adjuvants, other excipients may be added. If the carrier is proteinaceous it may be advantageous to induce cross-β structures in at least part of said carrier too. One or more of the antigenic components of the subunit vaccine may be coupled to said carrier. Again the antigenic components may be present as monomers, dimers, multimers, in head to tail arrangements (with or without spacers in between), or in other multimeric arrangements (known as “trees”, or “stars” and the like). The immunogenic compositions produced by the methods of the invention are also part of the present invention. Antigenic compositions according to the invention are typically the known vaccines against the known desired antigens, to which at least one proteinaceous compound is added in an amount of up to 30 weight percent of the antigenic material present in the vaccine. In a preferred embodiment the cross-β structure providing compounds are present in 10-30 weight % in relation to the antigenic material. When the cross-β structure comprising material of the invention is used to replace a conventional carrier in a vaccine, it may be used in approximately the same weight ratios as the original carrier. When part of the relevant antigen(s) is used to provide the cross-β structure compounds, then the total amount of antigenic material should preferably be at least the same as in a vaccine without said cross-β structures. Typically this means that the total amount of antigen (normal+cross-β structure comprising) will be 10 to 50, preferably 5-30% higher then the vaccine without cross-β structures. Of the antigen to be used as cross-β structure providing compound typically at least 50% is denatured, more preferably greater than 90% is denatured. The optimal combination of denatured and normal antigen for each vaccine is determined through simple rising dose studies. Ranges of antigenic compositions are produced which comprise 5, 10, 20, 30, 40 50, weight % of denatured antigenic material to determine the proper amount of added cross-β structure containing antigens. When the range is determined it is fine tuned by making a range in between the best doses. The same is done when inert proteinaceous material is used to provide the cross-β structures.

The intended use of the antigenic compositions according to the invention is as vaccines, be it therapeutic or prophylactic. The preferred use is in prophylaxis against infectious agents. The vaccine field is an old field of art. Persons of skill in this art are very well capable of adapting the present invention to known vaccines. In addition, vaccines (e.g. subunit vaccines) which lacked sufficient efficacy (protection) can be enhanced by the methods and means of the present invention.

Thus in a further embodiment the invention provides the use of cross-β structures in the preparation of a vaccine for the prophylaxis of an infectious disease, or more preferably the use of cross-β structures induced in a protein component of an infectious agent in the preparation of a vaccine inducing an immune response against said infectious agent, in particular the use above, wherein said protein component is a viral or bacterial protein and wherein said infectious agent is a virus, or a bacterium.

In another embodiment the invention provides a subunit vaccine comprising at least one viral protein, wherein at least 4-50%, preferably 10-30% of said viral protein is in a conformation comprising cross-β structures.

In yet another embodiment the invention provides a subunit vaccine comprising at least one bacterial protein, wherein at least 4-50%, preferably 10-30% of said bacterial protein is in a conformation comprising cross-β structures.

In yet another embodiment the invention provides a subunit vaccine comprising at least two viral proteins, wherein at least 4-50%, preferably 10-30% of at least one of said viral proteins is in a conformation comprising cross-β structures.

In yet another embodiment the invention provides a subunit vaccine comprising at least two bacterial proteins, wherein at least 4-50%, preferably 10-30% of at least one of said bacterial proteins is in a conformation comprising cross-β structures.

In a further embodiment the invention provides a use of cross-β structures in the preparation of an immunogenic composition for the prophylaxis and/or treatment of cancer. Said immunogenic composition is preferably a vaccine for the prophylaxis and/or treatment of a tumour or metastasis. Cross-β structures induced in a protein component of a vaccine are preferably used for inducing an immune response against a tumour or metastasis. Said protein component preferably comprises a tumour antigen. Hence, induction of cross-β structures in a tumour antigen is particularly suitable for production of an immunogenic composition capable of eliciting an immune response against said tumour. Alternatively, or additionally, said protein component is combined with another compound comprising cross-β structures. Said other compound preferably comprises an adjuvant. Preferably use is made of ovalbumin wherein the formation of cross-β structures has been induced and/or enhanced.

In one preferred embodiment a method according to the invention is used for preparing an immunogenic composition against a tumour which is induced by an infectious agent, such as for instance a virus. Most preferably, cross-β structures are used in the preparation of an immunogenic composition against a Human papillomavirus (HPV)-related tumour. Preferably, cross-β structures are induced and/or enhanced in an HPV E6 protein and/or HPV E7 protein. Such HPV E6 protein and/or HPV E7 protein wherein the formation of cross-β structures has been induced and/or enhanced is particularly suitable for eliciting an immune response against an HPV-related tumour. Alternatively, or additionally, an HPV E6 protein and/or HPV E7 protein is combined with another compound comprising cross-β structure. Said other compound preferably comprises an adjuvant. Preferably use is made of ovalbumin wherein the formation of cross-β structures has been induced and/or enhanced.

In yet another embodiment the invention provides a use of cross-β structures in the preparation of an immunogenic composition for immuno-castration. In this embodiment the formation of cross-β structures is preferably induced and/or enhanced in LHRH. Alternatively, or additionally, an LHRH is combined with another compound comprising cross-β structure. Said other compound preferably comprises an adjuvant.

A use of cross-β structures in the preparation of an immunogenic composition for the prophylaxis and/or treatment of atherosclerosis, amyloidoses, autoimmune diseases, graft-versus-host rejections and/or transplant rejections is also herewith provided. Said immunogenic composition preferably comprises a vaccine. Cross-β structures are preferably induced in a protein component of a vaccine capable of inducing an immune response against a protein component involved in at least one of the above mentioned diseases, preferably atherosclerosis, amyloidoses and/or an auto-immune disease, wherein said protein component is an antigen and wherein said disease is associated with accumulation of said protein component. In yet another embodiment the invention provides the use of cross-β structures in the preparation of an immunogenic composition, preferably a vaccine, for inducing an immune response in the prophylaxis or treatment of other aberrancies, as well as for inducing an immune response against any other moiety or self antigen, preferably, but not limited to, nicotine, haptens and/or LHRH. In one embodiment cross-β structures are induced in a protein component of a vaccine capable of inducing an immune response against components involved in graft versus host (GvH) or transplant rejections.

In yet another embodiment the invention provides an immunogenic composition comprising a bacterial or parasitic or viral antigen, said antigen comprising at least between 4-50%, preferably 10-30%, of said antigen in a cross-β structure conformation. Said antigen preferably comprises HPV E6 protein, HPV E7 protein, Influenza haemaglutinin H5, Influenza haemaglutinin H7, pestivirus E2 protein, Fasciola hepatica CL3 protein and/or Neisseria PorA protein. Said immunogenic composition preferably is a vaccine.

A method according to the invention for producing an immunogenic composition and/or for improving immunogenicity of a composition, said composition comprising at least one peptide, polypeptide, protein, glycoprotein and/or lipoprotein wherein said at least one peptide, polypeptide, protein, glycoprotein and/or lipoprotein, comprises HPV E6, HPV E7, Fasciola hepatica CL3, Influenza H5, Influenza H7, pestivirus E2 protein and/or Neisseria PorA protein, is also herewith provided.

According to the present invention immunogenicity of a protein or peptide is increased after inducing and/or enhancing formation of cross-β structures in said protein. Said protein for instance comprises β2glycoprotein I, which is a self-protein. Increase in immunogenicity of self-proteins is very useful for the induction of an immune response against such proteins, which are normally not easily recognised by the immune system as antigens. Examples of such proteins are for instance LHRH, β2glycoprotein I, and tumour antigens. Inducing and/or enhancing cross-β structure conformation in such protein or antigenic peptide thereof results in an (enhanced) immune response upon administration of said protein or antigenic peptide to an animal or human.

In one aspect the invention provides an immunogenic composition comprising a β2glycoprotein I or an antigenic peptide thereof, said immunogenic composition comprising at least between 4-67%, preferably 10-33% of said protein or peptide in a cross-β structure conformation. Another embodiment provides an immunogenic composition comprising a β2glycoprotein I or an antigenic peptide thereof, wherein said β2glycoprotein I, or an antigenic peptide thereof, is coupled to or mixed with another protein or peptide thereof comprising at least between 4-67%, preferably 10-33% of said another protein or peptide in a cross-β structure conformation. In one embodiment an immunogenic composition is provided which comprises a β2glycoprotein I or an antigenic peptide thereof, wherein said immunogenic composition comprises at least between 4-67%, preferably 10-33% of said β2glycoprotein I protein or peptide in a cross-β structure conformation and wherein said β2glycoprotein I or antigenic peptide is coupled to or mixed with another protein or peptide wherein at least between 4-67%, preferably 10-33% of said another protein or peptide is in a cross-β structure conformation. Such immunogenic compositions are preferably used as a vaccine. In one preferred embodiments said immunogenic compositions are used for the prophylaxis or treatment of an autoimmune disease.

In yet another embodiment the invention provides an immunogenic composition comprising a bacterial or parasitic or viral protein or an antigenic peptide thereof, said protein comprising at least between 4-67%, preferably 10-33% of said protein or peptide in a cross-β structure conformation. An immunogenic composition comprising a bacterial or parasitic or viral protein or an antigenic peptide thereof wherein said protein or antigenic peptide is coupled to or mixed with another protein or peptide thereof comprising at least between 4-67%, preferably 10-33% of said other protein or peptide in a cross-β structure conformation is also herewith provided. One preferred embodiment provides an immunogenic composition comprising a bacterial or parasitic or viral protein or an antigenic peptide thereof, said protein comprising at least between 4-67%, preferably 10-33% of said protein or peptide in a cross-β structure conformation, wherein said protein or antigenic peptide is coupled to or mixed with another protein or peptide wherein at least between 4-67%, preferably 10-33% of said other protein or peptide is in a cross-β structure conformation. The above mentioned immunogenic compositions preferably comprises a vaccine. Said another protein preferably comprises OVA or KLH or a combination of both, since these compounds are particularly well capable of enhancing immunogenicity. The invention therefore further provides an immunogenic composition and/or vaccine according to the present invention, wherein said another protein comprises OVA or YLH or a combination of both.

An immunogenic composition or vaccine according to the invention further comprising an adjuvant is also herewith provided. An adjuvant further enhances immunogenicity.

It is clear that the vaccines according to the invention comprise all kinds of subunit vaccines known, whether they comprise proteins from one or more infectious agents, epitopes from one or more agents (or combinations of epitopes and proteins from one or more agents), optionally with other antigenic compounds (polysaccharides, lipids, LPS, DNA, oligodeoxynucleotides (ODN), ODN-CpG),), or complexes including proteins from one or more agents. It is clear that vaccines according to the invention comprise all kinds of vaccines, including vaccines for prophylaxis of infections caused by, but not limited to virus, bacteria, fungi, yeast, or parasites.

The invention in one embodiment provides compositions which are essentially non-immunogenic with desired immunogenicity. In another embodiment the invention provides known immunogenic compositions with improved or enhanced immunogenicity.

Thus in a further embodiment the invention provides a method for improving immunogenicity of a composition comprising at least one peptide, polypeptide, protein, glycoprotein and/or lipoprotein, comprising contacting at least one of said peptide, polypeptide, protein, glycoprotein and/or lipoprotein with a cross-β structure inducing agent, thereby providing said composition with additional cross-β structures.

In particular the invention aims at improving the immunogenicity of known vaccines. Thus in a further embodiment the invention provides a method for enhancing immunogenicity of a vaccine composition comprising at least one peptide, polypeptide, protein, glycoprotein and/or lipoprotein, comprising contacting at least one of said peptide, polypeptide, protein, glycoprotein and/or lipoprotein with a cross-β structure inducing agent, thereby providing said vaccine composition with additional cross-β structures. In order to determine whether a vaccine composition can be improved in immunogenicity by providing said composition with further cross-β structures, one determines the amount of cross-β structures already present therein by means as disclosed herein, particularly by binding with a cross-β structure binding compound, such as Congo red or Thioflavin T staining. In a preferred manner said amount of cross-β structure is determined by binding of a cross-β structure binding compound, such as listed in Table 1-3, preferably tPA or factor XII, and detecting the amount of bound cross-β structure in a manner known per se and determining whether adding further cross-β structures improves the immune response.

Thus the invention further provides a method for determining the amount of cross-β structures in a vaccine composition, comprising contacting said vaccine composition with at least one cross-β structure binding compound and relating the amount of bound cross-β structures to the amount of cross-β structures present in the vaccine composition.

The invention will be illustrated in further detail in the following experimental section.

### EXAMPLES 1-5

### Experimental Procedures

### Plasminogen Activation Assay, Factor XII Activation Assay and Factor XII/Prekallikrein Activation Assay.

Plasmin (Plm) activity was assayed as described 1. Peptides and proteins that were tested for their stimulatory ability were used at 100 μg ml⁻¹, unless stated otherwise. Tissue-type plasminogen activator (tPA, Actilyse, Boehringer-Ingelheim) and plasminogen (Plg, purified form human plasma by lysine-affinity chromatography) were used at concentrations of 400 pM and 1.1 or 0.22 μM, respectively. Chromogenic substrate S-2251 (Chromogenix, Instrumentation Laboratory SpA, Milano, Italy) was used to measure Pls activity. To determine tPA activating properties of adjuvants, 1600 pM tPA and 0.22 μM Plg were mixed with the following adjuvants: 5 μg/ml dextran-sulphate MW=500 kDa (DXS500k, Pharmacia, Sweden), 20× diluted complete Freund's adjuvant (CFA, DIFCO, Brunswig, #0368-60), 2.1 μg/ml CpG (Coley Pharmaceutical Group, M A, USA), 1% v/v alum suspension (Imject, Pierce, Rockford, Ill., USA) with 0.5% v/v pooled citrated human plasma, or 100 μg/ml dimethyl dioctadecyl ammonium bromide (DDA, Sigma, D2779) suspension. Concentrations are the final adjuvant concentrations used in the assay.

Conversion of the zymogen factor XII (#233490, Calbiochem, EMD Biosciences, Inc., San Diego, Calif.) to proteolytically active factor XII (factor XIIa) was assayed indirectly by measurement of the conversion of chromogenic substrate Chromozym-PK (Roche Diagnostics, Almere, The Netherlands) by kallikrein formed by factor XIIa cleavage of prekallikrein. Chromozym-PK was used at a concentration of 0.3 mM. Factor XII, human plasma-derived prekallikrein (#529583, Calbiochem) and the cofactor for the reaction, human plasma-derived high-molecular weight kininogen (#422686, Calbiochem) were used at concentrations of 1 μg ml⁻¹. The assay buffer contained HBS (10 mM HEPES, 4 mM KCl, 137 mM NaCl, pH 7.2, 5 μM ZnCl₂, 0.1% m/v BSA (A7906, Sigma, St. Louis, Mo., USA)). Assays were performed using microtiter plates (#2595, Costar, Cambridge, Mass., USA or Exiqon peptide/protein Immobilizer, Vadbaek, Denmark). Peptides and proteins were tested for their ability to activate factor XII. 150 μg ml⁻¹ kaolin, an established activator of factor XII, was used as positive control and solvent ((H₂O) as negative control. The conversion of Chromozym-PK was recorded kinetically at 37° C. In control wells factor XII was omitted from the assay solutions.

Alternatively, activation of factor XII was measured directly using chromogenic substrate S-2222 (Chromogenix). Activation of factor XII in plasma was measured using 60% v/v plasma, diluted with substrate and H₂O with or without potential cofactor. Auto-activation of purified factor XII was measured by incubating 53 μg ml⁻¹ purified factor XII in 50 mM Tris-HCl buffer pH 7.5 with 1 mM EDTA and 0.001% v/v Triton-X100, with S-2222 and H₂O, with or without potential cofactor.

### Binding of tPA to Cross-β Structure Conformation Containing Protein Aggregates

Binding of tPA to amyloid-like aggregates was determined with ELISAs. Aggregates with cross-β structure conformation were immobilized on Exiqon (Vadbaek, Denmark), Nunc (amino strips, catalogue #076901) Immobilizer plates or Greiner microlon high-binding plates (Greiner Bio-One, The Netherlands). Binding of tPA was detected with a monoclonal antibody 374b (American Diagnostica, Tebu-Bio, The Netherlands). K2P-tPA, a tPA analogue that lacks the N-terminal F-EGF-like domain-kringle 1 domain (Reteplase, Boehringer-Ingelheim, Germany), was used as control. Binding of tPA and K2P-tPA was tested in the presence of 10 mM ε-amino caproic acid (eACA), a lysine analogue that abolishes the binding of the tPA kringle2 domain to solvent exposed lysine residues.

### Preparation of Amyloid-Like Aggregates and Control Peptide Solutions

Amyloid preparations of human γ-globulins were made as follows. Lyophilized γ-globulins (G4386, Sigma-Aldrich) were dissolved in a 1(:)1 volume ratio of 1,1,1,3,3,3-hexafluoro-2-propanol and trifluoro-acetic acid and subsequently dried under an air stream. Dried γ-globulins were dissolved in H₂O to a final concentration of 1 mg ml⁻¹ and kept at room temperature for at least three days. Aliquots were stored at −20° C.

Other peptide batches with amyloid-like properties were prepared as follows. Peptides used were human Aβ(1-40) Dutch type (DAEFRHDSGYEVHHQKLVFFAQDVGSNKGAIIGLMVGGVV), amyloid fragment of transthyretin (TTR11, YTIAALLSPYS), laminin α1-chain (2097-2108) amyloid core peptide (LAM12, AASIKVAVSADR), mouse non-amyloidogenic IAPP (20-29) core (mIAPP, SNNLGPVLPP), non-amyloid fragment FP10 of human fibrin α-chain (148-157) (KRLEVDIDIK)¹ and human fibrin α-chain (148-160) amyloid fragment with Lys157Ala mutation (FP13, KRLEVDIDIAIRS) (BB, unpublished and ^{1,2}). Aβ, IAPP, FP13 and LAM12 were disaggregated in a 1:1 (v/v) mixture of 1,1,1,3,3,3-hexafluoro-2-isopropyl alcohol and trifluoroacetic acid, air-dried and dissolved in H₂O (Aβ, IAPP, LAM12: 10 mg ml⁻¹, FP13: 1 mg ml⁻¹). After three days at 37° C., peptides were kept at room temperature for two weeks, before storage at 4° C. Freshly dissolved Aβ (10 mg ml⁻¹) in 1,1,1,3,3,3-hexafluoro-2-isopropyl alcohol and trifluoroacetic acid was diluted in H₂O prior to immobilization on ELISA plates. TTR11 (15 mg ml⁻¹) was dissolved in 10% (v/v) acetonitrile in water, at pH 2 (HCl), and kept at 37° C. for three days and subsequently at room temperature for two weeks. mIAPP and FP10 were dissolved at a concentration of 1 mg ml⁻¹ in H₂O and stored at 4° C. Peptide solutions were tested for the presence of amyloid conformation by Thioflavin T-(ThT, #T3516, Sigma-Aldrich, St. Louis, Mo., USA) or Congo red fluorescence as described³⁻⁵. Congo red was from Aldrich Chemical Company (#86, 095-6, Milwaukee, Wis., USA).

### Thioflavin T Fluorescence

Fluorescence of ThT-amyloid-like protein/peptide adducts was measured as follows. Solutions of 25 μg ml⁻¹ of protein or peptide preparations were prepared in 50 mM glycine buffer pH 9.0 with 25 μM ThT. Fluorescence was measured at 485 nm upon excitation at 435 nm. Background signals from buffer, buffer with ThT and protein/peptide solution without ThT were subtracted from corresponding measurements with protein solution incubated with ThT. Regularly, fluorescence of Aβ was used as a positive control, and fluorescence of FP10, a non-amyloid fibrin fragment¹, and buffer was used as a negative control. Fluorescence was measured in triplicate on a Hitachi F-4500 fluorescence spectrophotometer (Hitachi, Ltd., Tokyo, Japan).

To determine the ThT-fluorescence inducing capacity of adjuvants, 80× or 20× diluted human plasma was incubated with an adjuvant and subsequently diluted 40 times before ThT fluorescence was determined. Diluted plasma and adjuvants were incubated for 30 min. at room temperature, before dilution in ThT assay buffer. DEAE-dextran (Pharmacia, 17-0350-01) was used at 5 μg ml⁻¹, DXS500k was used at 5 μg ml⁻¹, DDA was used at 1 μg ml⁻¹. Specol (7925000, ID-DLO, The Netherlands) was diluted at a 5:4 ratio with 40× or 10× diluted plasma. CFA, incomplete Freund's adjuvant (IFA) and alum suspension were used at a 1:1 ratio with 40× or 10× diluted plasma. CpG was used at a concentration of 11 μg ml⁻¹. Experiments with 80× diluted plasma included a vortexing step during mixing of diluted plasma with all adjuvants, except alum. Alum was mixed with plasma by rolling on a roller bank for 30 min. When 20× diluted plasma was used, diluted plasma was mixed by swirling with DXS500k, DEAE-dextran, DDA and CpG, whereas plasma and adjuvant were mixed by vortexing for 20 sec when CFA, IFA, Specol or alum were used. CpG at a concentration of 10.7, 21.4 and 42.8 μg ml⁻¹ was subsequently incubated for 30 min. at RT or o/n at 4° C., at a rollerbank with 1 mg ml⁻¹ lysozyme or endostatin. Enhancement of ThT fluorescence was measured similarly as described above.

Alternatively, CpG at 21.4 μg ml⁻¹ was mixed with 1 mg ml⁻¹ of chicken egg-white lysozyme (Fluka, #62971), bovine serum albumin (ICN, #160069, fraction V), recombinant human collagen XVIII fragment endostatin (Entremed, Inc, Rockville, Md.), human γ-globulins, plasma human β2-glycoprotein I (see below) and recombinant human β2-glycoprotein I (see below), and incubated o/n on a roller at 4° C., before ThT fluorescence measurements. For this purpose, protein solutions at 2 mg ml⁻¹ were ultracentrifuged for 1 h at 100,000*g before use, and subsequently diluted 1:1 in buffer with 42.9 μg ml⁻¹ CpG. Also transmission electron microscopy (TEM) images are taken with the CpG, CpG with lysozyme, lysozyme samples. In addition, lysozyme was incubated with 250 μg ml⁻¹ DXS500k and TEM images are recorded with lysozyme with DXS500k and with DXS500k alone.

### Activation of tPA by β₂-Glycoprotein I, Binding of Factor XII and tPA to β₂-Glycoprotein I and ThT and TEM Analysis of β₂-Glycoprotein I

Purification of β2-glycoprotein I (β₂GPI) was performed according to established methods^{6,7}. Recombinant human β₂GPI was made using insect cells and purified as described⁶. Plasma derived β₂GPI as used in a factor XII ELISA, the Plg-activation assay and in the anti-phospholipid syndrome antibody ELISA (see below), was purified from fresh human plasma as described⁷. Alternatively, β₂GPI was purified from, either fresh human plasma, or frozen plasma using an anti-β₂GPI antibody affinity column⁸.

Activation of tPA (Actilyse, Boehringer-Ingelheim) by β₂GPI preparations was tested in a Plg-activation assay (see above). Hundred μg ml⁻¹ plasma β₂GPI or recombinant β₂GPI were tested for their stimulatory activity in the Plg-activation assay and were compared to the stimulatory activity of peptide FP13¹.

Binding of human factor XII from plasma (Calbiochem) or of recombinant human tPA to β₂GPI purified from human plasma, or to recombinant human β₂GPI was tested in an ELISA. Ten μg of factor XII or tPA in PBS was coated onto wells of a Costar 2595 ELISA plate and overlayed with concentration series of β₂GPI. Binding of β₂GPI was assessed with monoclonal antibody 2B2⁸. Binding of factor XII to β₂GPI was also tested using immunoblotting. β₂GPI (33 μg) purified either from fresh plasma or from frozen plasma loaded onto a 7.5% poly-acrylamide gel. After blotting to a nitrocellulose membrane (Schleicher & Schuell), the blot was incubated with 1000× diluted rabbit polyclonal anti-human factor XII antibody (#233504, Calbiochem) and after washing with 3000× diluted peroxidase-coupled swine anti-rabbit immunoglobulins (SWARPO, #P0217, DAKO, Denmark).

ThT fluorescence of β₂GPI was measured as follows. Purified β₂GPI from human plasma (400 μg ml⁻¹ final concentration) was incubated with or without 100 μM cardiolipin vesicles or 250 μg ml⁻¹ of the adjuvant DXS500k, in 25 mM Tris-HCl, 150 mM NaCl, pH 7.3. In the ThT fluorescence assay, fluorescence of β₂GPI in buffer, of cardiolipin or DXS500k in buffer, of buffer and ThT alone, and of β₂GPI-cardiolipin adducts and β₂GPI-DXS500k adducts, with or without ThT, was recorded as described above (section ThT fluorescence). In addition, transmission electron microscopy (TEM) images were recorded with cardiolipin, β2GPI from human plasma, with or without cardiolipin, and with recombinant β2GPI, as described³.

### Interference with Binding of Anti-β₂GPI Autoantibodies from Antiphospholipid Syndrome Autoimmune Patients to Immobilized β₂GPI by Recombinant β₂GPI and not by Plasma Derived β₂GPI

When plasma derived β₂GPI is coated onto hydrophilic ELISA plates, anti-β₂GPI auto-antibodies isolated from plasma of antiphospholipid syndrome autoimmune patients can bind¹⁹. To study the influence of coincubations of the coated β₂GPI with the antibodies together with plasma β₂GPI or recombinant β₂GPI, concentration series of β₂GPI were added to the patient antibodies. Subsequently, binding of the antibodies to coated β₂GPI was determined.

### Analysis of Protein Structure after Exposure to Adjuvants

Lyophilized proteins were dissolved in HEPES-buffered saline (HBS, 10 mM HEPES, 4 mM KCl, 137 mM NaCl, pH 7.2) to a final concentration of 2 mg ml⁻¹. Proteins were gently dissolved on a roller at room temp. for 10 min, at 37° C. for 10 min and again at room temp. for 10 min. Kaolin (6564, Genfarma, Zaandam, The Netherlands) suspension and DXS500k stock solutions of 500 μg ml⁻¹ were prepared in HBS. Albumin (ICN, 160069), lysozyme (ICN, 100831), γ-globulins (G4386, Sigma-Aldrich, Zwijndrecht, The Netherlands), endostatin (EntreMed, Inc., Rockville, Md.) and factor XII (Calbiochem, 233490) were diluted 1:1 in HBS alone or in HBS with kaolin or DXS500k. Human pooled citrated plasma was diluted 40× in HBS before use to obtain an estimated total protein concentration of 2 mg ml⁻¹, and subsequently diluted 1:1 in buffer or adjuvant solution/suspension. Control protein samples and the protein-adjuvant samples were incubated overnight at 4° C. on a roller. After incubation, 25 μl of the samples were analyzed for ThT binding (see above). Fluorescence of the buffer or the adjuvants was recorded for background subtraction purposes. Amyloid-β(1-40) E22Q was used as a positive control. Alternatively, control proteins and proteins incubated with the soluble adjuvant DXS500k were immobilized on Greiner microlon high-binding ELISA plates. Wells were blocked with Blocking reagent (Roche). Glycated haemoglobin (Hb-AGE) was immobilized as a positive control for tPA binding to a protein aggregate with amyloid-like properties. Hb-AGE i) appears as fibrous structures under the transmission electron microscope (not shown), ii) contains an increased amount of β-sheet secondary structure, as determined with circular dichroism spectropolarimetry (not shown), and iii) enhances Congo red fluorescence (not shown). Samples were overlayed with concentration series of full-length tPA or K2P-tPA, in the presence of 10 mM εACA.

### ThT Fluorescence Analysis of Lysozyme Structure after Exposure to Lipopolysaccharide

Lipopolysaccharide (LPS) binds to lysozymel¹⁰, which can prevent biological activities of LPS¹¹, and LPS activates factor XII¹². We tested whether binding of lysozyme is accompanied by a conformational change in the protein with introduction of amyloid like structure. For this purpose 0, 10, 25, 100, 200, 600 and 1200 μg ml⁻¹ LPS (from Escherichia coli serotype 011:B4, #L2630, lot 104K4109, Sigma-Aldrich) was incubated overnight at 4° C. or for 30 min. at room temp. on a roller with 1 mg ml⁻¹ lysozyme (ICN, 100831) in HBS. Subsequently, the ability to enhance ThT fluorescence was determined with 40× diluted solution, as described above.

Alternatively, similarly as described above for CpG, LPS at 600 μg ml⁻¹ was mixed with 1 mg ml⁻¹ of lysozyme, albumin, endostatin, γ-globulins, plasma β2-glycoprotein I (β2GPI) and recombinant β2-GPI, and incubated o/n on a roller at 4° C., before ThT fluorescence measurements. Again, protein solutions at 2 mg ml⁻¹ were ultracentrifuged for 1 h at 100,000*g before use, and subsequently diluted 1:1 in buffer with 1200 μg ml⁻¹ LPS.

### Activation of U937 Monocytic Cells by LPS and Cross-β Structure Conformation Comprising Polypeptides

U937 monocytes were cultured in six-wells plates. Cells were stimulated with buffer (negative control), 1 μg ml⁻¹ LPS (positive control), 100 μg ml⁻¹ amyloid endostatin^{1,3}, 260 μg ml⁻¹ glycated haemoglobin and 260 μg ml⁻¹ control haemoglobin. After 1 h of stimulation, cells were put on ice. After washing RNA was isolated and quantified spectrophotometrically. Normalized amounts of RNA were used for 26 cycli of RT-PCR with human TNFα primer and 18 cycli of RT-PCR with ribosomal 18S primer for normalization purposes. DNA was analyzed on a 2% agarose gel.

### Preparation of Amyloid-Like Ovalbumin, Human Glucagon, Etanercept and Murine Serum Albumin

To prepare structurally altered ovalbumin (OVA) with amyloid cross-β structure conformation, purified OVA (Sigma, A-7641, lot 071k7094) was heated to 85° C. One mg ml⁻¹ OVA in 67 mM NaPᵢ buffer pH 7.0; 100 mM NaCl, was heated for two cycles in PCR cups in a PTC-200 thermal cycler (MJ Research, Inc., Waltham, Mass., USA). In each cycle, OVA was heated from 30 to 85° C. at a rate of 5° C./min. Native OVA (nOVA) and heat-denatured OVA (dOVA) were tested in the ThT fluorescence assay and in the Plg-activation assay. In the fluorescence assay and in the Plg-activation assay, 25 and 100 μg ml⁻¹ nOVA and dOVA were tested, respectively. TEM images of nOVA and dOVA were taken to check for the presence of large aggregates. Modified murine serum albumin (MSA) was obtained by reducing and alkylation. MSA (#126674, Calbiochem) was dissolved in 8 M urea, 100 mM Tris-HCl pH 8.2, at 10 mg ml⁻¹ final concentration. Dithiothreitol (DTT) was added to a final concentration of 10 mM. Air was replaced by N₂ and the solution was incubated for 2 h at room temperature. Then, the solution was transferred to ice and iodoacetamide was added from a 1 M stock to a final concentration of 20 mM. After a 15 min. incubation on ice, reduced-alkylated MSA (alkyl-MSA) was diluted to 1 mg ml⁻¹ by adding H₂O. Alkyl-MSA was dialyzed against H₂O before use. Native MSA (nMSA) and alkyl-MSA were tested in the ThT fluorescence assay and in the Plg-activation assay. In the ThT-fluorescence assay 25 μg ml⁻¹ nMSA and alkyl-MSA were tested, and in the Plg-activation assay 100 μg ml⁻¹ was tested. Presence of aggregates or fibrils was analyzed with TEM.

Amyloid-like properties in human glucagon (Glucagen, #PW60126, Novo Nordisk, Copenhagen, Denmark) were introduced as follows. Lyophilized sterile glucagon was dissolved at 1 mg ml⁻¹ in H₂O with 10 mM HCl. The solution was subsequently kept at 37° C. for 24 h, at 4° C. for 14 days and again at 37° C. for 9 days. ThT fluorescence was determined as described above, and compared with freshly dissolved glucagon. tPA-activating properties of both heat-denatured glucagon and freshly dissolved glucagon was tested at 50 μg ml⁻¹. TEM analysis was performed to assess the presence of large multimeric structures.

### Immunization of Balb/c Mice with Ovalbumin and Amyloid-Like Ovalbumin

Eight to ten weeks old female Balb/c mice are immunized with OVA according to two immunization regimes (Central Animal Laboratories, Utrecht University, The Netherlands). Pre-immune serum was collected prior to the immunizations. In one regime two groups of five mice are subcutaneously injected five consecutive days per week, for three consecutive weeks. Doses comprised 10 μg native OVA or heat-denatured OVA for each injection. Alternatively, according to the second protocol, three groups of five mice are injected once intraperitoneally with doses comprising 5 μg nOVA, 5 μg OVA or 5 μg native OVA mixed 1:1 with complete Freund's adjuvant. Each week, blood was taken. After three weeks, a second dose was given. Incomplete Freund's adjuvant was used instead of complete Freund's adjuvant. Blood was taken after one week after the start of the immunization. Antibody titers in sera were determined and sera were analyzed for the presence of cross-β structure conformation specific antibodies. For this purpose, nOVA was coated onto wells of 96-wells ELISA plates and incubated with dilution series of sera. Sera of the groups of five mice were pooled prior to the analyzes. Plates were washed and subsequently incubated with peroxidase-coupled rabbit anti-mouse immunoglobulins (RAMP0, P0260, DAKOCytomation, Glostrup, Denmark). Plates were subsequently developed with tetramethylbenzidine (TMB) substrate. The reaction was terminated with H₂SO4.

### RESULTS EXAMPLES 1-5

### Results Example 1

### Factor XII is Activated by Negatively Charged Surfaces and by Peptides with Cross-β Structure Conformation

### Activation of Factor XII by Protein Aggregates with Amyloid-Like Cross-β Structure Conformation

It is known that contacting factor XII to artificial negatively charged surfaces, such as kaolin and DXS results in its activation. Here, we demonstrate that peptide aggregates with cross-β structure conformation also stimulate factor XII activation, as measured by the conversion of prekallikrein to kallikrein, which can convert chromogenic substrate Chromozym-PK. (FIG. 1A, B). We also show the ability of protein aggregates with cross-β structure conformation to induce auto-activation of factor XII (FIG. 1C). For this purpose, purified factor XII was incubated with substrate S-2222 and either buffer, or 1 μg ml⁻¹ DXS500k, 100 μg ml⁻¹ FP13 K157G, 10 μg ml⁻¹ Aβ(1-40) E22Q and 10 μg ml⁻¹ Hb-AGE. All three amyloid-like aggregates are able to induce factor XII auto-activation. FP13 K157G and Hb-AGE have a potency to induce auto-activation that is similar to the established surface activator DXS500k, whereas the potency of the Aβ(1-40) E22Q is somewhat lower.

### Results Example 2

### Adjuvants Introduce Amyloid-Like Properties in Proteins

### Adjuvants Act as Denaturants and Induce Cross-β Structure Conformation in Proteins

Factor XII and tPA bind to protein or peptide aggregates with amyloid-like cross-β structure conformation^{1,3,13} and unpublished results B Bouma/MFBG Gebbink. Furthermore, binding to cross-β structure containing aggregates results in activation of both serine proteases (See example 1 and ¹. In addition, binding of ThT to amyloid-like protein conformations results in a specific fluorescent signal. Moreover, aggregation of peptides and proteins with cross-β structure conformation can finally result in formation of fibrillar or amorphous precipitates which can be visualized with transmission electron microscopy (TEM). These methods were therefore used to determine whether exposure of a protein or peptide to various adjuvants that are used in vaccination regimes, introduces amyloid-like properties.

We hypothesized that at least part of adjuvant function and activity may reside in the ability to introduce cross-β structure conformation or any other amyloid-like conformation, either in the antigen, or in any other protein or peptide contacting the adjuvant. Alternatively, peptide- or protein based adjuvants may have amyloid-like properties themselves. The amyloid-like protein conformation is then the immunogenic factor that induces an immune response.

To test this hypothesis, purified albumin, γ-globulins, lysozyme, factor XII, endostatin and diluted plasma were exposed to kaolin or DXS500k, two compounds that are well known for their ability to activate FXII but are also used as adjuvant. Subsequently, ThT fluorescence was determined. Factor XII was only exposed to DXS500k. After subtraction of background signals, kaolin induces an increased ThT fluorescence signal of 1.6 up to 6.6 fold. DXS500k enhances ThT fluorescence 2.6 times (factor XII) to 17.8 times (albumin) (FIG. 2A). In an ELISA binding of tPA and K2P-tPA to immobilized control proteins and mixtures of proteins with DXS500k was assessed (FIG. 2A). KP-tPA did not bind to any of the proteins or DXS500k-protein mixtures (not shown). Exposure of proteins or diluted plasma to DXS500k increased tPA binding with a factor 1.3 (albumin) up to 10.5 (endostatin), when compared to the binding of tPA to proteins that were incubated with buffer only. The ThT fluorescence data and the tPA binding data indicate that exposure of proteins to adjuvants kaolin and DXS500k induces or enhances amyloid-like properties in proteins.

Next, the role of amyloid-like cross-β structure conformation on factor XII activation was assessed. For this purpose amyloid fibrin peptide FP13 K157G, an effective activator of factor XII (FIG. 1C), was incubated with purified factor XII, in the presence of activated factor XII substrate S2222, and with or without ThT (FIG. 2B). The results show that ThT, a dye with established affinity for amyloid-like aggregates, effectively inhibits the stimulatory activity of FP13 K157G (FIG. 2B). This provides direct evidence for a role of the cross-β structure conformation in the activation of factor XII. For already a long time, compounds such as glass, kaolin, DXS500k, surgeon steel, platinum and ellagic acid are known for their ability to induce factor XII activity. The current view is that factor XII is activated by specific interaction of the protein with negative charges. Based on our observations that various amyloid-like aggregates are also able to activate factor XII, we hypothesized that negatively charged compounds activate factor XII in an indirect way, through cross-β structure conformation formed in proteins exposed to negatively charged surfaces. Thus factor XII activating compounds, including adjuvants, serve as denaturing agents that induce protein/peptide aggregation accompanied by the formation of amyloid-like properties. To test this hypothesis, we used assay conditions during which factor XII is not or hardly activated by DXS500k (FIG. 2C). Under these conditions factor XII can be activated by adding 80× diluted plasma. Activation is fully inhibited by introducing ThT. These observations indicate that the denatured (plasma) proteins comprising cross-β structure conformation are the true activators of factor XII, rather than the negative charge itself. In FIG. 2D we show that yet another adjuvant, Ca₃(PO₄)₂, is an activator of factor XII. When sufficient amounts of factor XII are used in the assay, no additional protein is necessary for activation. We also established that factor XII itself can obtain amyloid-like conformation upon exposure to adjuvants (FIG. 2A). Thus, autoactivation of factor XII can now be explained by the fact that denatured cross-β structure containing and perhaps aggregated factor XII at the surface of a negatively charged surface can serve as the activating substance for other factor XII molecules. Besides diluted plasma or elevated levels of factor XII we found that albumin and endostatin can be used (FIG. 2E, F). Neither albumin or endostatin alone, nor kaolin or DXS500k alone are efficient activators of factor XII, whereas combinations of adjuvant and protein cofactor results in factor XII and subsequent prekallikrein activity. Taken together, activation of factor XII requires (1) a denaturing surface and (2) sufficient amounts of a protein that is capable of denaturing on the provided surface.

We next tested whether negatively charged surfaces and adjuvants could also induce activation of tPA. The adjuvants DXS500k, CFA, CpG, alum and DDA all turn out to be activators of tPA (FIG. 2G, H). Under the tested conditions, i.e. 1600 pM tPA, 0.22 μM Plg, only alum requires an additional protein cofactor (diluted plasma) for its tPA-activating property. Likely, with the other adjuvants tPA and/or Plg itself partly denature on the adjuvant surface, thereby inducing formation of the amyloid cross-β structure conformation that can subsequently activate tPA.

The same adjuvants together with IFA, Specol and DEAE-dextran, were also analyzed for their ability to induce ThT fluorescence upon incubation with 80× or 20× diluted plasma (FIGS. 2I and 2J, respectively). With 80× diluted plasma, CFA, Specol, DXS500k and CpG induce ThT fluorescence, and with 20× diluted plasma, CFA, Specol, DXS500k, CpG, as well as IFA induce ThT fluorescence, indicative for the formation of amyloid-like protein aggregates with cross-β structure conformation. Furthermore, CpG at 10.7, 21.4 and 42.8 μg ml⁻¹ incubated overnight with 1 mg ml⁻¹ lysozyme enhanced ThT fluorescence with a factor 1.1, 1.2 and 1.4, respectively, further indicative for the denaturing capacity of CpG (not shown). In addition, when 10.4 or 21.7 μg ml⁻¹ CpG is incubated with 1 mg ml⁻¹ lysozyme or endostatin for 30 min. at room temp., an increase in ThT fluorescence of approximately 8 to 7 times for lysozyme and 39 to 56 times for endostatin is observed, respectively (FIG. 2K, L). In addition, exposure of 1 mg ml⁻¹ albumin, endostatin, plasma β2GPI or rec. β2GPI to 21.4 μg ml⁻¹ CpG results in increased ThT fluorescence with approximately a factor 3, 10, 2 and 5, respectively (FIG. 2M). With these assay conditions no effect is seen with lysozyme and γ-globulins. Analysis with TEM of CpG, lysozyme and lysozyme with CpG, all after overnight incubation, revealed that small needles are present in the CpG solution (FIG. 2N) and a few aggregates are present in the lysozyme solution (FIG. 2O). When CpG and lysozyme are incubated together, a high density of relatively thick aggregates are observed that seem to be composed of strings of globular precipitates (FIG. 2P). A large amount of even larger networks of similar strings of globular aggregates are seen with lysozyme exposed to DXS500k needles (FIG. 2Q, R). The needles in the CpG and DXS500k solutions disappeared after exposure to lysozyme.

Further analyses by means of circular dichroism spectropolarimetry, Fourier Transform infrared spectroscopy, transmission electron microscopy, binding studies with cross-β structure conformation binding compounds, proteins and protein fragments and X-ray fiber diffraction studies could add additional information on the presence of amyloid-like aggregates with cross-β structure conformation in proteins and peptides that are exposed to adjuvants. In principle, any established adjuvant or any newly discovered adjuvant can be screened for its denaturing capacity, accompanied by formation of aggregates with cross-β structure conformation, or for the presence of amyloid-like protein conformation in the adjuvant itself. Immunization trials with wild type species or transgenic species, or cell-based immune assays with antigens combined with denaturing adjuvants, or with antigen alone, or with denatured antigen comprising cross-β structure conformation will reveal whether adjuvants act as inducers of an immune response by their capacity to induce aggregation accompanied with cross-β structure conformation. Perhaps, adjuvants are not strictly required, that is to say, an antigen with cross-β structure conformation may be immunogenic by itself. To test this view, a comparison can be made between immunizations with 1) native antigens, with 2) antigens with cross-β structure conformation or with an adjuvant that induces or comprises cross-β structure conformation, and with 3) native antigens combined with a conventional adjuvant such as CFA, Specol, alum, LPS or derivatives thereof, and CpG. Immunization trials with mice or with in vitro cell-based assays can for example be performed with 1) native OVA, glucagon, albumin or plasma β2GPI, 2) heat-denatured OVA, heat/acid-denatured glucagon, heat-denatured albumin, alkylated albumin, recombinantly produced β2GPI, plasma β2GPI together with DXS500k or cardiolipin, and 3) CFA with native OVA, glucagon, albumin or β2GPI. These experiments will also contribute to the understanding of the working mechanism of the class of CpG-like adjuvants. These adjuvants transmit their immunogenic activity via Toll-like receptor 9, via a poorly understood mechanism. Direct interaction between CpG and TLR9 has not been demonstrated so far. Our results suggest that a denatured protein is required. This protein is preferably denatured by CpG. The role of cross-β structure conformation in the potentiation of immunogenicity by CpG can now be easily tested by a person skilled in the art. If true, the immunopotentiation of CpG should be diminished in the presence of an inhibitor of cross-β structure formation or by an inhibitor of the interaction of the cross-β structure conformation with one of its target molecules that transduces the immunogenic signal. Such inhibitor could be any cross-β structure binding compound, such as ThT, tPA or an equivalent thereof, an antibody against the relevant cross-β structure conformation or an antibody against the target receptor. The target receptor could be any of the multiligand receptors that bind or possibly bind cross-β structure comprising proteins, such as tPA, factor XII, fibronectin, hepatocyte growth factor activator, CD14, low density lipoprotein receptor like protein, CD36, scavenger receptors A, scavenger receptors B, Toll-like receptors and receptors for advanced glycation endproducts.

### Results Example 3

### Relationship Between the Structure of β2-Glycoprotein I, the Kev Antigen in Patients with the Antiphospholipid Syndrome, and Antigenicity.

### The Anti-Phospholipid Syndrome and Conformationally Altered β₂-Glycoprotein I

The anti-phospholipid syndrome (APS) is an autoimmune disease characterized by the presence of anti-β₂-glycoprotein I auto-antibodies. Two of the major clinical concerns of the APS are the propensity of auto-antibodies to induce thrombosis and the risk for fetal resorption. Little is known about the onset of the autoimmune disease. Recent work has demonstrated the need for conformational alterations in the main antigen in APS, β₂-glycoprotein I (β₂GPI), before the initially hidden epitope for auto-antibodies is exposed ¹⁴. Binding of native β₂GPI to certain types of ELISA plates mimics the exposure of the cryptic epitopes that are apparently present in APS patients¹⁴. It has been demonstrated that anti-β₂GPI autoantibodies do not bind to globular β₂GPI in solution, but only when β₂GPI has been immobilized to certain types of ELISA plates¹⁴. The globular (native) form of the protein is not immunogenic, but requires the addition of cardiolipin, apoptotic cells or modification by oxidation¹⁵⁻¹⁶. Thus the generation of autoantibodies seems to be triggered by and elicited against a conformationally altered form of β₂GPI. It has previously been proposed that the induction of an adaptive immune response requires a so-called “danger” signal, which among other effects stimulates antigen presentation and cytokine release by dendritic cells¹⁷. The following results imply that cardiolipin induces cross-β structure conformation in β2GPI which than serves as a danger signal. In analogy other negatively charged phospholipids, or structures that contain negatively charged lipids, such as liposomes or apoptotic cells, or other inducers of cross-β structure conformation, including LPS, CpG that possess cross-β structure conformation inducing properties, may be immunogenic due to the fact, at least in part, that they induce cross-β structure conformation.

### Factor XII and tPA Bind to Recombinant β₂GPI and to β₂GPI Purified from Frozen Plasma, But not to β₂GPI Purified from Fresh Plasma

Recombinant β₂GPI, but not β₂GPI purified from fresh plasma stimulate tPA-mediated conversion of Plg to plasmin, as measured as the conversion of the plasmin specific chromogenic substrate S-2251 (FIG. 3A). Using an ELISA it is shown that tPA and factor XII bind recombinant β₂GPI, but not bind to β₂GPI purified from fresh human plasma (FIG. 3B, C). Recombinant β₂GPI binds to factor XII with a k_{D} of 20 nM (FIG. 3C) and to tPA with a k_{D} of 51 nM (FIG. 3B). In addition, β₂GPI purified from plasma that was frozen at −20° C. and subsequently thawed, factor XII co-elutes from the anti-β₂GPI antibody affinity column, as shown on Western blot after incubation of the blot with anti-factor XII antibody (FIG. 3D). This suggest that β₂GPI refolds into a conformation containing cross-β structure upon freezing. In FIG. 3E, the inhibitory effect of recombinant β₂GPI on binding of anti-β₂GPI autoantibodies isolated from patients with APS to immobilized β₂GPI is shown. It is seen that plasma derived β₂GPI in solution has hardly an effect on the antibody binding to immobilized β₂GPI. FIG. 3F shows that exposure of β₂GPI to cardiolipin or DXS500k introduces an increased ThT fluorescence signal, indicative for a conformational change in β₂GPI accompanied with the formation of cross-β structure conformation. Again, recombinant β₂GPI initially already gave a higher ThT fluorescence signal than native β₂GPI purified from plasma. In addition, exposure of plasma β2GPI and rec. β2GPI to adjuvants/denaturants LPS or CpG also induces an increase in ThT fluorescence, which is larger with rec. β2GPI than with plasma β2GPI for both adjuvants (FIG. 2M and FIG. 4C). These data not only indicate that recombinant β2GPI already comprises more cross-β structure conformation than plasma β2GPI, but that recombinant β2GPI also adopts more readily this conformation when contacted to various adjuvants and surfaces, i.e. cardiolipin, DXS500k, LPS and CpG. In FIG. 3G it is shown that exposure of β₂GPI to cardiolipin, immobilized on the wells of an ELISA plate, renders β₂GPI with tPA binding capacity. Binding of β₂GPI directly to the ELISA plate results in less tPA binding. These observations also show that cardiolipin has a denaturing effect, thereby inducing amyloid-like conformation in β₂GPI, necessary for tPA binding. These observations, together with the observation that exposure of β₂GPI to cardiolipin vesicles induced ThT binding capacity (FIG. 3F), show that exposure of β₂GPI to a denaturing surface induces formation of amyloid-like cross-β structure conformation.

### Epitopes for Autoantibodies are Specifically Exposed on Non-Native Conformations of β₂GPI Comprising Cross-β Structure Conformation

FIG. 3 shows that preparations of β₂GPI react with amyloid cross-β structure markers ThT, tPA and factor XII. In addition, exposure of β₂GPI to cardiolipin introduces tPA binding capacity (FIG. 3G). Furthermore, large fibrillar structures are seen on TEM images of plasma β₂GPI in contact with cardiolipin (FIG. 3H, image 2 and 3). Small cardiolipin vesicles seem to be attached to the fibrillar β2GPI. Images of plasma β₂GPI alone (FIG. 3H, image 1) or cardiolipin alone (not shown) revealed that no visible ultrastructures are present. In contrast, non-fibrillar aggregates and relatively thin curly fibrils can be seen on images of recombinant β₂GPI (FIG. 3H, image 4). These observation show that exposure of β₂GPI to cardiolipin and expression and purification of recombinant β₂GPI result in an altered multimeric structure of β₂GPI, when compared to the monomeric structure observed with X-ray crystallography¹⁸. The β₂GPI preparations with cross-β structure conformation express epitopes that are recognized by anti-β₂GPI auto-antibodies isolated from APS patient plasma. Furthermore, exposure of β₂GPI to cardiolipin or DXS500k induces an increased fluorescence when ThT is added, indicative for the formation of cross-β structure conformation when β₂GPI contacts a negatively charged surface. Interestingly, it has previously been observed that exposure of β₂GPI to cardiolipin is a prerequisite for the detection of anti-β₂GPI-antibodies in sera of immunized mice ¹⁵. These combined observations point to a role for conformational changes in native β₂GPI, necessary to expose new immunogenic sites. Our results indicate that the cross-β structure element is part of this epitope. We predict that the cross-β structure conformation can be relatively easily formed by one or more of the five domains of the extended β₂GPI molecule¹⁸. Each domain comprises at least one β-sheet that may function as a seed for local refolding into cross-β structure conformation. A person skilled in the art is now able to test the hypothesis that the cross-β structure conformation is the essential to elicit anti-β₂GPI antibodies. Immunization studies with native β₂GPI and conformationally altered β₂GPI, with or without cross-β structure conformation, can be performed in the presence or absence of a compound, including ThT, tPA, RAGE, CD36, anti-cross-β structure antibodies or a functional equivalent thereof, that inhibits the activity of cross-β structure conformation. Alternatively, in vitro studies with antigen presenting cells (APC), including dendritic cells (DC) can be performed. Sources of conformationally altered β₂GPI are recombinant β₂GPI, or β₂GPI exposed to any denaturing surface, e.g. plastics, cardiolipin, DXS500k and potentially other adjuvants. In addition, structurally altered β₂GPI may be obtained by any other chemical or physical treatment, e.g. heating, pH changes, reduction-alkylation. A person skilled in the art is able to design and perform in vitro cellular assays and in vivo mouse models to obtain further evidence for the role of the cross-β structure conformation in autoimmunity (see below). To establish whether the cross-β structure element is essential for eliciting an immune response or for antibody binding, inhibition studies can be conducted with any cross-β structure binding compound that may compete with antibody binding or that may prevent an immune response.

Our observations indicate that cross-β structure conformation is necessary for the induction of an adaptive immune response. The cross-β structure conformation could also be part of an epitope recognized by autoimmune antibodies. Based on our studies it is expected that other diseases and complications in which autoantibodies are implicated are mediated by a protein comprising cross-β structure conformation. In addition to the antiphospholipid syndrome such conditions include, but are not limited to systemic lupus erythematosus (SLE), type I diabetes, red cell aplasia and the formation of inhibitory antibodies in haemophilia patients treated with factor VIII. A person skilled in the art is now able to screen haemophilia patients with antifactor VIII autoantibodies for the presence of antibodies in their plasma that recognize the cross-β structure conformation. A more detailed analysis will reveal whether putative cross-β structure binding antibodies specifically bind (in part) to cross-β structure conformation in the antigen, or whether the antibodies bind to cross-β structure conformation present in any unrelated protein.

### Results Example 4

### Incubation of Cultured U937 Monocytes with Proteins Comprising Cross-β Structure Conformation Results in Upregulation of Tissue Necrosis Factor-α mRNA Levels, and LPS Induces Formation of Amyloid-Like Structures in Lysozyme.

### Cross-β Structure Rich Compounds Induce Expression of TNFα RNA in Monocytes

After exposure of U937 monocytes to LPS or cross-β structure rich amyloid endostatin or Hb-AGE, TNFα DNA is obtained after RT-PCR with isolated RNA (FIG. 4A). Control haemoglobin does induce TNFα RNA upregulation to some extent but does not exceed approximately 30% of the values obtained after amyloid endostatin or glycated Hb stimulation. Amounts of TNFα DNA obtained after RT-PCR with monocyte RNA are normalized for the amounts of ribosomal 18S DNA present in the corresponding samples.

### LPS Acts as a Denaturant and Induces Cross-β Structure Conformation

After exposure of 1 mg ml⁻¹ lysozyme to 10, 25, 100, 200, 600 and 1200 μg ml⁻¹ LPS in solution, ThT fluorescence is enhanced 1.1, 1.3, 1.6, 2.3, 5.7 and 13.1 times respectively when compared to lysozyme incubated in buffer only, indicative for the formation of amyloid-like conformation with cross-β structure (FIG. 4B). When lysozyme and endostatin are exposed to 200, 400 and 600 μg ml-1 LPS, ThT fluorescence is enhanced approximately 5, 11 and 18 times and 8, 20 and 26 times, respectively (FIG. 2K, L). Alternatively, similar to what is observed with CpG (FIG. 2M), when 1 mg ml⁻¹ lysozyme, albumin, γ-globulins, endostatin, plasma β2GPI or rec. 82GPI are exposed to 600 μg ml-1 LPS, ThT fluorescence is enhanced approximately 10, 3, 2, 10, 2 and 4 times, respectively (FIG. 4C). Additional TEM imaging could shed further light on whether the LPS exposed proteins have rearranged their conformation into amyloid like fibrils or into other visible aggregates. The ThT fluorescence enhancement data show that LPS acts as a denaturant that converts an initially globular protein into an amyloid-like polypeptide. Previously, it has already been demonstrated that lysozyme can bind to purified LPS and to complete Freund's adjuvant, comprising bacterial cell wall fragments with LPS, accompanied by structural changes in the protein. Furthermore, Morrison & Cochrane¹² showed that LPS can potently activate factor XII, which adds to our view that LPS acts as a protein denaturant, which in turn introduces factor XII activating properties (see also FIG. 2E, F). Our results now disclose that LPS binding induces cross-β structure conformation and that LPS activation of factor XII is mediated by protein with cross-β structure conformation, providing an explanation for these previously reported observations.

### Discussion: Similar to LPS, Cross-β Structure Rich Proteins Induce TNFα Upregulation in Monocytes, and LPS Induces Amyloid Cross-β Structure Conformation in Lysozyme

Stimulation of U937 monocytes with proteins that comprise cross-β structure conformation as part of their tertiary/quarternary fold results in expression of TNFα RNA, similar to the upregulation of TNFα RNA by LPS. The observation that control haemoglobin did influence TNFα RNA levels only to some extent indicates that the presence of cross-β structure conformation is an important factor for the observed upregulation. Since we here show that LPS acts as a cross-β structure conformation-inducing agent we conclude that the activation of cells, including cells of the immune system, by LPS is induced, at least in part, by a conformationally altered protein comprising cross-β structure conformation. Thus, LPS acts as a denaturing surface or adjuvant that induces cross-β structure conformation formation in a protein that is present on the cell surface or in the cell environment, similar to our observation that LPS introduces amyloid-like cross-β structure conformation in lysozyme. The formed cross-β structure conformation is than a stimulator of the immune response. Our results, hypothesis and conclusions are supported by the observations in literature that the endotoxic activity of LPS is enhanced in the presence of albumin or haemoglobin. Moreover, LPS induces formation of β-sheets in albumin, a structural element that is absent in the albumin native fold and which suggests that cross-β structure conformation is formed¹⁹. Similar responses of microglial cells towards LPS and aggregated Aβ are reported²⁰. Our observations give a rationale to these and recent additional observations that the LPS receptor CD14 is involved in Aβ phagocytosis^{21,22}. In the light of our results CD14 perhaps interacts with a denatured protein associated with LPS and with Aβ via a similar non-native protein conformation in the ligands. This would suggest that CD14 is a possible member of the class of amyloid-like cross-β structure binding proteins³. For a person skilled in the art, these observations provide the means to perform additional in vitro cell assays that support the role of cross-β structure conformation on activation of the immune system. A person skilled in the art can now select the appropriate cellular assays, to gather insight in the type of immune response induced by cross-β structure conformation. For example, the potency to activate the host innate and/or adaptive immune system and to induce a cellular and/or a humoral immune response can be tested. Even at a more detailed level, the type of response, i.e. a T-cell helper 1 type of response resulting in eliciting immunoglobulins of the IgG2a subclass, or a T-cell helper 2 type of response primarily resulting in eliciting IgG1, or a T-cell regulatory type of response. Blocking experiments using cross-β structure binding compounds and proteins, e.g. ThT, Congo red, Thioflavin S (ThS), tPA and fragments thereof, factor XII and fragments thereof, anti-cross-β structure hybridomas, can provide further evidence for the role of the cross-β structure element in the activation of the immune system. Furthermore, cellular assays can be used to study which appearance of the cross-β structure conformation bears the immunogenic nature, i.e. soluble oligomers, fibrils, or other appearances. Cellular immune assays can also be used to screen established and new adjuvants for their ability to induce an immune response, mediated by cross-β structure conformation, in the adjuvant itself or induced by the adjuvants (See FIG. 2). Again, pretreatment of adjuvants/protein mixtures with potentially neutralizing cross-β structure binding compounds or proteins may prevent an immune response.

Further insight into the role of the denaturing capacity of LPS in induction of an immune response can come from comparative studies in which endotoxic active and inactive variants of LPS are tested for their capacity to introduce cross-β structure conformation in proteins and for the effects on the immune system. Examples of endotoxic inactive LPS are Rhodobacter capsulatus LPS and tetra- or penta-acylated lipid A¹⁹. In addition, the effect of Polymyxin B, which inhibits the endotoxic activity of LPS, on the cross-β structure-inducing properties of LPS can be studied. Alternatively, Polymyxin B may act directly on cross-β structure containing proteins. In that case polymyxin B is added to the list of cross-β structure binding compounds.

Our results indicate that the potentiating effects of LPS, when it is used as an adjuvant in immunization experiments, are attributed at least in part by the introduction of immunogenic cross-β structure conformation in the administered antigen, in a co-administered or in an endogenous protein or set of endogenous proteins. It is now predicted that determination of the endogenous protein(s) that preferentially form the cross-β structure conformation upon exposure to LPS will provide a tool for the design of safer immunization regimes. It is predicted that LPS can be reduced or omitted when these endogenous protein or set of denatured proteins in which the cross-β structure conformation is introduced is used directly as the adjuvant. For a person skilled in the art it is clear that these results and conclusions can also be obtained with other adjuvants, including, but not limited to CpG or Alum.

### Results Example 5

### Immunization of Mice in the Absence of Adjuvant with Polypeptides Comprising Cross-β Structure Conformation.

### Preparation of Antigens with Cross-β Structure Conformation

The data in Example 2 and Example 4 show that various adjuvants used in animal and human vaccination regimes induce the cross-β structure conformation in proteins. The presence of cross-β structure conformation in various protein therapeutics induces immunogenicity. This suggest that immunogenicity may be attributed, at least in part, to cross-β structure comprising proteins or polypeptides. This prompted us to set up immunization trials with cross-β structure conformation rich compounds, without addition of an adjuvant. Based on the results described above it is predicted that the presence of the immunogenic cross-β structure conformation is essential and even sufficient to induce an immune response, such as for example seen with various protein-based pharmaceuticals that lack an adjuvant. Indeed higher antibody titers we obtained when we used chicken OVA with cross-β structure conformation (dOVA) in comparison with OVA without cross-β structure conformation (nOVA) in immunization experiments (FIG. 5L). Titers were also obtained with OVA without cross-β structure conformation. Since the formation of cross-β structure in OVA can readily occur it is predicted that the generation of antibodies after immunization with nOVA is also mediated by molecules with cross-β structure conformation. In this case the cross-β structure conformation is induced during or after the subcutaneous injection. These experiments establish that the presence of the cross-β structure conformation in a protein can induce immunogenicity that can be harmful. In the case of a protein therapeutic, removing or diminishing the cross-β structure content of the therapeutic will aid to a safer medicine.

Amyloid-like OVA was obtained by heat denaturation at 85° C. (FIG. 5A, B, I, K). The presence of the cross-β structure conformation was established with ThT fluorescence and Plg-activation assays and by TEM imaging. The fibrillar structures of at least up to 2 μm in length, seen on the TEM images are likely not the only OVA assemblies with cross-β structure conformation present, as concluded from the observation that filtration through a 0.2 μm filter does not reduce the enhancement of ThT fluorescence. A person skilled in the art can perform similar experiments with murine serum albumin, human glucagon and Etanercept stock solutions with the cross-β structure conformation, such as those described below (FIG. 5).

The amyloid-like protein fold was induced in albumin by heat denaturation at 85° C. and by reduction and alkylation of disulphide bonds (FIG. 5A-D). We observed that also native albumin enhanced ThT fluorescence to some extent, but this was not reflected by stimulation of tPA activation. Although heat-denatured albumin and alkylated albumin enhance ThT fluorescence to a similar extent, they differ in tPA activating potential. This suggests that tPA and ThT interact with distinct aspects of the cross-β structure conformation. Previously, we observed that Congo red, another amyloid-specific dye, can efficiently compete for tPA binding to amyloid-like aggregates in ELISAs, whereas ThT did not inhibit tPA binding at all (patent application WO 2004/004698).

Amyloid-like cross-β structure conformation was induced in glucagon by heat-denaturation at 37° C. at low pH in HCl buffer (FIG. 5E, F, J). In this way, a potent activator of tPA was obtained, that enhanced ThT fluorescence to a large extent. In addition, long and bended unbranched fibrils are formed, as visualized on TEM images (FIG. 5J). Noteworthy, at high glucagon concentration, also native glucagon has some tPA activating potential, indicative for the presence of a certain amount of cross-β structure conformation rich protein.

Alkylated Etanercept does not activate tPA at all, whereas heat-denatured Etanercept has similar tPA activating potential as amyloid γ-globulins (FIG. 5G). After heat denaturation, Etanercept also efficiently induces enhanced ThT fluorescence (FIG. 5H). Native Etanercept both induces some tPA activation and gave some ThT fluorescence enhancement.

For immunizations of Balb/c mice, nOVA, dOVA and nOVA with complete Freund's adjuvant were used. Similar immunizations and analyzes can be performed with n-MSA, heat-denatured MSA, alkyl-MSA, native glucagon, heat-denatured glucagon, native Etanercept, denatured Etanercept, native β2GPI, alkyl-β2GPI, denatured β2GPI, recombinant β2GPI, dimer β2GPI²³, β2GPI together with CpG, β2GPI together with cardiolipin and β2GPI together with DXS500k. Furthermore, the analysis of the various titers may point to improved immunization protocols with respect to dose, number of injections, way of injection, pre-treatment of the antigen to introduce more immunogenic cross-β structure conformation.

For example, 25 μg Etanercept, heat-denatured Etanercept, glucagon and heat/acid-denatured glucagon will be administered subcutaneously without adjuvant at day 0 and at day 18. Blood for titer determinations will be drawn from the vena saphena at day −3, day 18 and day 25. Native β2GPI (15 μg), reduced/alkylated β2GPI (15 μg) and native β2GPI (15 μg) with 1.35 μg cardiolipin will be administered intravenously at day 0, day 4, day 14 and day 18. The β2GPI and cardiolipin will be premixed and incubated at 400 μg ml⁻¹ and 25 μM final concentrations. Blood will be drawn at day −3, day 9, day 25. At first, titers will be determined with ELISA's using plates coated with the native proteins.

From our analyzes we conclude that β2GPI with cardiolipin, dOVA, alkyl-MSA, heat/acid-denatured glucagon and heat-denatured Etanercept comprise the cross-β structure conformation. The presence of the cross-β structure conformation can be further established by circular dichroism spectropolarimetry analyzes, X-ray fiber diffraction experiments, Fourier transform infrared spectroscopy, Congo red fluorescence/birefringence, tPA binding, factor XII activation and binding, and more.

### Assessing Immunogenicity of Compounds with Cross-β Structure Conformation with a ‘Whole Blood’ Assay

One way of assessing whether a protein with cross-β structure conformation is activating cells of the immune system is by use of a ‘whole blood’ assay. For this purpose, at day 1 freshly drawn human EDTA-blood is added in a 1:1 ratio to RPMI-1640 medium (HEPES buffered, with L-glutamine, Gibco, Invitrogen, Breda, The Netherlands), that is prewarmed at 37° C. Subsequently, proteins comprising cross-β structure conformation can be added. Preferably a positive control is included, preferably LPS. An inhibitor that can be used for LPS is Polymyxin B, 5 μg ml⁻¹ final concentration. Standard cross-β structure conformation rich polypeptides that can be tested are Aβ, amyloid γ-globulins, glycated proteins, FP13, heat-denatured OVA and others. Negative controls are native γ-globulins, native albumin, native Hb, freshly dissolved Aβ or FP13, native OVA. As a control, all protein samples can be tested in the absence or presence of 5 μg ml⁻¹ Polymyxin B to exclude effects seen due to endotoxin contaminations. In addition, native proteins alone or pre-exposed to denaturing adjuvants, e.g. LPS, DXS500K, kaolin and CpG, or new adjuvants, can be tested for immunogenic activity. The blood and the medium should be mixed carefully and incubated overnight in a CO₂ incubator with lids that allow for the entrance of CO₂. At day 2 medium will be collected after 10′ spinning at 1,000*g, at room temperature. The cell pellet will be stored frozen. The medium will again be spinned for 20′ at 2,000*g, at room temperature. Supernatant will be analyzed using ELISAs for concentrations of markers of an immune response, e.g. tissue necrosis factor-α. When positive and negative controls are established as well as a reliable titration curve, any solution can be tested for the cross-β structure load with respect to concentrations of markers for immunogenicity. Furthermore, putative inhibitors of the immune response can be tested. For example, finger domains, ThT, Congo red, sRAGE and tPA may prevent an immune response upon addition to protein therapeutic solutions comprising aggregates.

### Immunogenicity of Proteins with Cross-β Structure

The present invention discloses that proteins containing cross-β structure conformation are immunogenic. For a person skilled in the art it is now evident that further evidence can be obtained that support the proposed role for the cross-β structure conformation in immunogenicity. For example the immunogenicity of proteins, including OVA, β2GPI and/or protein therapeutics such as tissue necrosis factor α, glucagon or Etanercept is tested. Preferably the immunogenicity of the native state of these proteins is compared with a state in which the cross-β structure conformation has been introduced. Preferably the cross-β structure conformation is induced by heating, oxidation, glycation or treatment with an adjuvant, such as CpG oligodeoxynucleotides, LPS or cardiolipin. The content of cross-β structure conformation is preferably measured by ThT, Congo red, TEM, size exclusion chromatography, tPA-activating activity, and or binding of any other cross-β structure binding protein listed in Tables 1-3. The immunogenicity of said protein is tested preferably in vitro and in vivo. For a person skilled in the art several in vitro assays are preferable to determine the immunogenicity of said protein. Preferably, activation of antigen presenting cells (APC), preferably dendritic cells (DC) is tested following treatment with said native or cross-β structure comprising protein. Preferably, this is performed according to established protocols. Activation of antigen presenting cells can be determined by FACS (Fluorescence Activated Cell Sorter) analysis. Preferably the levels of so-called co-stimulatory molecules, such as B7.1, B7.2, MHC class II, CD40, CD80, CD86 are determined on preferably CD11c positive cells. Alternatively, activation of NF-κB and/or expression of cytokines can be used as indicators of activation of cells involved in immunogenicity, such as APC and DC.

Preferably, the following cytokines should be quantified: TNFα, IL-1, IL-2, IL-6, or IFNγ or other. Preferably, the cytokine levels should be quantified by ELISA. Alternatively, the mRNA levels can be quantified. For a person skilled in the art it is evident that function of APC and DC can be tested as well. Preferably the cross-presentation of antigen can be tested. Preferably this can be achieved using OVA, in its native conformation and conformations with cross-β structure conformation, as model protein. The ability of DC or APC to activate MHC class I-restricted or MHC class II-restricted T-cells should be analyzed. For a person skilled in the art this can be done according to established protocols ^{43,44}. The role of proteins with cross-β structure conformation in the activation of APC and their role in antigen presentation can be further addressed with these aforementioned experimental procedures using cross-β structure binding compounds in competition assays. Preferably DC activation and functional antigen presentation are tested in the presence or absence of ThT, Congo red, tPA, or any other cross-β structure binding protein, including those listed in Table 1-3 or a functional equivalent thereof. The immunogenicity of proteins with cross-β structure conformation is further demonstrated in vivo. For example the induction of antibodies and the induction of cytotoxic T lymphocyte (CTL) activity upon immunization of proteins, including OVA, β2GPI and/or protein therapeutics such as tissue necrosis factor α, glucagon or Etanercept is tested. Preferably the immunogenicity of the native state of these proteins is compared with a state in which the cross-β structure conformation has been introduced. Preferably the cross-β structure conformation is induced by heating, oxidation, glycation or treatment with an adjuvant, such as CpG oligodeoxynucleotides, LPS or cardiolipin. The content of cross-β structure conformation is preferably measured by ThT, Congo Red, TEM, size exclusion chromatography, tPA-activating activity, and or binding of any other cross-β structure binding protein listed in Tables 1-3. Preferably the antibody titers are measured after immunization by ELISA and the CTL activity is measured using ⁵¹Cr-release assay. Alternatively the release of cytokines, including IL-2 can be measured. For a person skilled in the art it is now clear that for each protein comprising cross-β structure conformation the effect on immunogenicity can be tested as such. These proposed experiments will further elucidate the role of the cross-β structure conformation in immunogenicity.

### Immunogenicity of Adjuvants

The present invention discloses that adjuvants induce cross-β structure conformation. For a person skilled in the art it is now evident that further evidence can be obtained that support the proposed role for the cross-β structure conformation in immunogenicity of adjuvants. For example additional and new adjuvants can be tested. For example Escherichia coli heat-labile enterotoxin (EtxB), different CpG-related oligodeoxynucleotides and/or variants of LPS, or LPS-related molecules, such as monophosphoryl lipid A (MPL). This cross-β structure-inducing capacity is measured using a native protein or set of proteins, preferably OVA, lysozyme, endostatin, γ-globulins, albumin, plasma or a plasma derived protein or set of proteins. The content of cross-β structure is preferably measured by ThT, Congo red, TEM, size exclusion chromatography, tPA-activating activity, and or binding of any other cross-β structure binding protein listed in Tables 1-3. Preferably the cross-β structure inducing capacity of these compounds is compared with the immunogenicity in vitro and in vivo using the assays described above. For a person skilled in the art it is now possible to identify the protein or set of proteins that refold into a conformation with cross-β structure upon exposure to an adjuvant. For example, an adjuvant, preferably CpG, LPS or a functional equivalent thereof is immobilized and subsequently used as adjuvant. Next the immobilized adjuvant is taken and after extensive washing the bound proteins with cross-β structure conformation are isolated. If needed the proteins can be further purified by standard procedures, preferably size-exclusion chromatography. The identity of the proteins is revealed preferably by mass spectrometry, i.e. mass spectrometry-based proteomics.

The role of cross-β structure conformation in the action of adjuvants is further addressed with these aforementioned experimental procedures using cross-β structure binding compounds in competition assays. Preferably DC activation and functional antigen presentation are tested in the presence or absence of ThT, Congo red, tPA, or any other cross-β structure binding protein, including those listed in Table 1-3 or a functional equivalent thereof.

These experiments further elucidate the role of cross-β structure conformation in immunogenicity. Moreover for a person skilled in the art it is feasible to select proteins that refold into cross-β structure conformation and become immunogenic upon binding to an adjuvant. Such proteins can than also be used as adjuvants themselves. Ultimately for a person skilled in the art it is possible to select the optimal cross-β structure comprising proteins for immunization and generation of an immune response based on the binding of cross-β structure binding compounds, including those listed in Table 1-3, to these cross-β structure comprising proteins.

### Immunization with Proteins Comprising Cross-β Structure Conformation Promote Protection Against Challenge with Pathogen

The present invention discloses that proteins containing cross-β structure conformation are immunogenic. For a person skilled in the art it is now evident that immunization with proteins comprising cross-β structure conformation induce or enhance protection against pathogens. For example pathogenic proteins that are good candidate components for vaccine development are combined with proteins comprising cross-β structure conformation. Such pathogenic proteins include, but are not limited to proteins involved in virulence of bacteria, such as M-like protein and fibronectin-binding proteins of Streptococcus species, or opacity (Opa) proteins of Neisseria meningitidis. Alternatively, such proteins are from viral origin, such as the haemagglutinin (HA) and/or neuraminidase (NA) protein of influenza virus. Such pathogenic proteins that elicit a protective immune response are combined with a protein comprising an effective amount of protein comprising cross-β structure conformation and injected in an animal, preferably a mouse. Alternatively the pathogenic proteins that induce a protective immune response are treated such that they refold into a conformation comprising cross-β structure. Such treatment preferably is heating, oxidation, and/or sonication or any other treatment that induces cross-β structure conformation. The content of cross-β structure conformation is preferably measured by ThT, Congo red, TEM, size exclusion chromatography, tPA-activating activity, and or binding of any other cross-β structure binding protein listed in Tables 1-3. After immunization the immune response can be easily determined by a person skilled in the art using established protocols for determination of antibody titer and induction of a CTL response. The protective effect of immunization with cross-β structure comprising proteins with a given pathogenic protein or set of pathogenic proteins that is expected to induce a protective immune response is analyzed by challenging immunized mice with the pathogen of which the pathogenic proteins are derived and compare the survival or severity of infection with mice that are not immunized. For example mice can be infected with Streptococcus equi after immunization with fibronectin binding proteins (FNZ and/or SFS) and/or EAG (a2-macroglobulin, albumin, and IgG binding protein) treated to induce cross-β structure conformation or combined with a protein comprising a cross-β structure conformation. Another example is obtained using immunization with recombinant meningococcal OpaB and OpaJ proteins or outer membrane vesicles containing PorA and PorB proteins treated to induce cross-β structure conformation or immunized with proteins comprising cross-β structure conformation. Using established protocols the mice are challenged with Neisseria meningitis. The effect of immunization is analyzed by determining the antibody response. In yet another example mice are immunized with recombinant baculovirus produced NA and NA vaccines treated to induce cross-β structure conformation or used in combination with a protein or set of proteins comprising cross-β structure conformation. Subsequently the mice are challenged with influenza virus to determine the protective effect of the immunization with cross-β structure comprising proteins for example according to established protocols.

The ideal protein or set of proteins to be used in combination with the pathogenic protein or set of proteins being preferably obtained from the analysis of proteins that are implicated in the response of adjuvants, such as CpG or LPS.

Taken together, the results of the experiments further illustrate that cross-β structure comprising proteins are valuable components of vaccines.

### EXAMPLE 6

Each time two doses of a commercially available inactivated influenza surface antigen vaccines (Agrippal®) are used to determine the relevant dose of cross-β structures.

One dose is used “as is” (A) and the other (B) is denatured by one or more treatments of heating, freezing, oxidation, glycation pegylation, sulphatation, exposure to a chaotroph, preferably the chaotroph is urea or guanidinium-HCl, phosphorylation, partial proteolysis, chemical lysis, preferably with HCl or cyanogenbromide, sonication, dissolving in organic solutions, preferably 1,1,1,3,3,3-hexafluoro-2-propanol and trifluoroacetic acid, or a combination thereof.

The following combinations are made.

| A % | B % |
|---|---|
| 50 | 50 |
| 60 | 40 |
| 70 | 30 |
| 80 | 20 |
| 90 | 10 |
| 100 | 0 |
| 100 | 10 |
| 100 | 20 |
| 100 | 30 |
| 100 | 40 |
| 100 | 50 |

Mice are immunized with Aβ combinations as described above, or with a placebo.

After three weeks mice are boosted with the same composition.

After another three weeks the mice are challenged with the pathogen.

The immune response is measured.

### EXAMPLE 7

Proteins comprising cross-β structure are also used as component for medical applications in the treatment and/or prophylaxis of cancer, preferably, but not limited to, cancer associated with viral infection. Said cancer is preferably associated with human papillomavirus (HPV) infection.

An immune response is for example induced in order to counteract, treat and/or at least partially prevent the occurrence and/or development of cancer, preferably cervical, anal, vulvar, vaginal, and/or penile cancers and/or genital warts associated with HPV infection, preferably cancer associated with HPV16 and/or HPV18 and/or HPV6 and/or HPV11 tumor-associated antigen. Preferably human papilloma virus (HPV) type E6 and/or E7 protein is targeted. Inducing cross-β structure in such antigen, preferably an E6 and/or E7 antigen, or combination of said antigen with a protein component comprising a cross-β structure, results in a compound and/or composition which is particularly suitable for eliciting an antigen-specific immune response. Preferably an E6 and/or E7-specific response is elicited. Preferably a compound and/or composition is produced which is capable of protecting mice against challenge with tumours expressing said antigen, preferably E6 and/or E7, Most preferably a compound and/or composition is produced which is capable of at least in part protecting humans from developing cancer and/or genital warts caused by HPV infection. Therapeutically effective amounts, preferably between 1 and 100 μg of tumour-antigen, are used in combination with therapeutically effective amounts, preferably 1-100 μg, of a protein component comprising cross-β structure. Said protein component comprising cross-β structure is preferably ovalbumin. Even more preferably, said protein component comprising cross-β structure is a tumour-associated antigen. Hence, cross-β structures are preferably induced in a tumour-associated antigen, which renders a composition comprising said tumour-associated antigen more immunogenic. If at least partial treatment and/or prophylaxis of HPV-associated cancers is desired, said tumour-associated antigen is preferably E6 and/or E7.

Mice are immunized, preferably intramuscular twice, preferably with an interval of two to three weeks and preferably challenged with tumour cells, preferably 5×10⁴ TC-1 tumour cells and the tumour growth is preferably measured and monitored in time. Human subjects are preferably immunized twice or more times in the same manner and the efficacy is preferably monitored by determining the number, onset and/or growth of cancer and/or development of warts between immunized individuals and non-immunized individuals.

### EXAMPLE 8

Proteins comprising cross-β structure are also used as component for medical application to induce immunogenicity in the prophylaxis and/or treatment of other aberrancies, preferably, but not limited to atherosclerosis or amyloidoses, preferably Alzheimer's diseases, as well as for inducing immune responses against other self antigens, as widely ranging as e.g. LHRH for immunocastration of boars, or for use in preventing graft versus host (GvH) and/or transplant rejections.

For example, to treat atherosclerosis preferably oxidized LDL or glycated proteins or specific epitopes thereof are targeted. Preferably, oxidized LDL and/or glycated proteins, comprising cross-β structure, are used as component of such a medical application to induce an ox-LDL- and/or glycated protein-specific immune response. Therapeutically effective amounts, preferably between 1 and 100 μg of ox-LDL and/or glycated protein, are used in combination with therapeutically effective amounts, preferably 1-100 μg, of a protein component comprising cross-β structure, wherein said protein component is preferably oxLDL and/or a glycated protein.

To treat amyloidosis or any protein misfolding disease, preferably Alzheimer's disease, preferably a protein or protein fragment combined with a particular amyloidoses is used as immunogen to induce a specific immune response against said protein or protein fragment. Preferably, therapeutically effective amounts, preferably between 1 and 100 μg, of said protein or protein fragment is used in combination with therapeutically effective amounts, preferably 1-100 μg, of a protein component comprising cross-β structure, wherein said protein component is preferably said protein or protein component.

To elicit an immune responses against a self antigen, as widely ranging as, for example, for immunocastration of boars, or for use in preventing graft versus host (GvH) and/or transplant rejections, preferably a self antigen is used as immunogen, preferably with a protein component comprising cross-β structure. Preferably said protein component comprising cross-β structure is said self antigen wherein cross-β structures have been induced. Preferably, therapeutically effective amounts, preferably between 1 and 100 μg, of said self protein is used in combination with therapeutically effective amounts, preferably 1-100 μg, of a protein component comprising cross-β structure, wherein said protein component comprising cross-β structure is preferably said self protein wherein cross-β structures have been induced. An elicited immune response is preferably determined by an immunological method, for instance by ELISA or determining CTL response. The efficacy of said treatment is for instance determined by monitoring the specific development of the targeted aberrancies.

### EXAMPLE 9

### Immunogenicity of Crossbeta Structure-Adjuvated Serogroup B Outer-Membrane Protein Meningococcal Vaccines

Immunization of mice with a trivalent PorA vaccine against Neisseria meningitidis

### Aim

Determine if PorA antigen with amyloid-like misfolded protein conformation elicits antibody titers. Mouse sera were analyzed for total antibody titers and bactericidal antibody titers against PorA from three different Neisseria meningitidis strains, after two vaccinations with alum-adjuvated PorA, native PorA, placebo (buffer for injection), or two PorA preparations with respectively 25% or 75% misfolded PorA with amyloid-like properties (crossbeta-structure adjuvated).

### Materials & Methods

### Preparation of Vaccines

PorA antigen solution was obtained from Dr G. Kersten and Dr G. van den Dobbelsteen from The Netherlands Vaccine Institute (NVI, Bilthoven, The Netherlands). PorA in bacterium outer membrane vesicles (OMV) was prepared essentially as described (1). Protein concentration in the OMV solution was determined using conventional techniques. The content of PorA was determined by densitometric analysis of a Coomassie-stained polyacryl-amide gel after gel-electrophoresis of the PorA preparation, and was 400 μg/ml. The PorA buffer is 10 mM Tris, 3% saccharose, pH 7.4. For vaccine preparation purposes, 10× PorA buffer (30% w/w sucrose, 100 mM Tris pH 7.4, 0.45 μm filter-sterilized) was prepared. The OMV used for the studies were obtained from a trivalent strain expressing three PorA serosubtypes, i.e. P1.5-2.10, P1.12-1.13 and P1.7-2.4. Adjuvant alum (Adju-Phos, Brenntag, 2% AlPO₄, 0.44% Al³⁺, Batch 8981) was supplied by NVI, as well as plain PorA buffer. These sterile stock solutions were stored at 4° C.

### Introduction of Amyloid-Like Misfolded Protein Conformation in Proteins

For preparation of amyloid-like misfolded PorA, the following three methods were used.

### Misfolding Method I: Heat Denaturation by Thermal Cycling

One mg of purified chicken OVA (Sigma; catalogue number A5503) in 875 μl PorA buffer and 125 μl PorA stock solution ([PorA]=50 μg/ml; [OVA]=1 mg/ml) was heated for five cycles in PCR cups in a PTC-200 thermal cycler (MJ Research, Inc., Waltham, Mass., USA). In each cycle, protein solution was heated from 30° C. to 85° C. at a rate of 5° C./min. Heat-denatured protein solutions were stored at −80° C.

### Misfolding Method II: Coupling of PorA to Ovalbumin

For coupling of PorA to OVA using N-ethyl-N′-(dimethylaminopropyl)carbodiimide (EDC, stock is 400 mM) and N-hydroxysuccinimide (NHS, stock is 100 mM), compounds and protein were mixed in MES buffer (0.02% (m/v) NaN₃, 100 mM MES, 150 mM NaCl, pH 4.7; diluted from a 10×MES buffer stock). In brief, 200 μl 10×MES buffer was added to 250 μl of EDC stock and 350 μl H₂O (solution 1). OVA (0.2 mg) was dissolved in 100 μl PorA stock solution. Eighty μl of solution 1 was added to this PorA/OVA solution; then, 20 μl of the NHS stock solutions was added. The mixtures were incubated for 2 h at room temperature on a roller device, and subsequently 800 μl PBS was added before extensive dialysis against PBS at 4° C. ([PorA]=40 μg/ml; [OVA]=1 mg/ml). In the solution with PorA−OVA conjugates, small particles were visible. The conjugate solution was subsequently heated for five thermal cycles as described above. The conjugate was stored at −80° C.

### Misfolding Method III: Coupling of Polypeptide-A to Polypeptide-β by Glutaraldehyde/NaBH₄ Activation

For coupling of PorA to OVA, both proteins were activated with glutaraldehyde and sodium-borohydride and mixed. For this purpose 100 μg OVA was dissolved in 250 μl PorA stock solution and 250 μl PorA buffer was added. Glutaraldehyde (25% (v/v) solution in H₂O, Merck, Hohenbrunn, Germany, 8.20603.1000 (UN2927, toxic), lot S4503603 549), pre-diluted to a 4% 100× stock in H₂O was added to a final concentration of 0.04%. After vortexing and a 2-minutes incubation at room temperature, 5 μl of a 120 mM 100× stock NaBH₄ (approx. 98%, Sigma, St. Louis, Mo., USA, S9125, lot 53H3475) was added to a final concentration of 1.2 mM. The solution was vortexed and incubated for 42 h at room temperature on a roller device. During this incubation small floating particles became visible. Then, the solution was extensively dialyzed against PBS. The conjugate solution was subsequently heated for five thermal cycles as described above. The final PorA and OVA concentrations were 200 μg/ml in PBS.

### Analysis of the Presence of Amyloid-Like Misfolded Protein with Crossbeta Structure in PorA Solutions

### Thioflavin T Fluorescence

To establish the enhancement of amyloid-specific dye Thioflavin T (ThT) fluorescence by PorA preparations, 90 μl of 25 μM ThT-solution in 50 mM Glycine buffer (pH 9.0) was added to 10 μl sample in duplicate wells of black 96-wells plates. Amyloid-β (Aβ) at a stock concentration of 1 mg/ml was used as a positive control. Fluorescence of duplicates was measured on a Thermo Fluoroskan Ascent 2.5, at 435 nm excitation and 485 nm emission wavelengths. In one assay, PorA and misfolded PorA after misfolding methods I-III were tested with 400-fold diluted PorA stock solutions. In a second assay, the five vaccine solutions (a-e) were tested at 20-fold dilution.

### Tissue-Type Plasminogen Activator—Plasminogen Activation Assay

Tissue-type plasminogen activator (tPA) binds to and is activated by amyloid-like misfolded protein. Activation of tPA results in conversion of its substrate plasminogen to plasmin, that can be followed in time using a chromogenic plasmin substrate. The assay was performed in 96-wells plates (Costar 2595 ELISA plates). The tPA-(Actilyse, Boehringer-Ingelheim) and plasminogen (Plg, purified from human plasma) concentrations were 400 pM and 0.2 μM, respectively. Chromogenic substrate S-2251 (Chromogenix, Milano, Italy), at 0.5 mM, was used to measure Plm activity. Assay buffer was HBS (10 mM HEPES, 4 mM KCl, 137 mM NaCl, pH 7.3). Negative control was H₂O, positive control was 20 μg/ml amyloid-like misfolded γ-globulins dissolved in H₂O. In one assay, PorA and misfolded PorA after misfolding methods I-III were tested with 400-fold diluted PorA stock solutions. In a second assay, the five vaccine solutions (a-e) were tested at 20-fold dilution. Samples were tested in duplicate wells, completed with a negative control well in which tPA was not added. The total assay volume was 50 μl. Kinetic readings were performed with a spectrophotometer (Spectramax) at 405 nm, and were taken each minute for 3 h, at 37° C., with shaking before each reading.

### Experimental Immunization Setup

Female 7-9 weeks-old BalB/CAnNHSd (BalB/C, Harlan) were housed in filtertop cages in five groups of five mice per group (Animal Facility ‘Gemeenschappelijk Dierenlaboratorium’, Utrecht University, The Netherlands). After approximately one week of adjustment to the environment, blood was drawn to collect pre-immune serum (day −3). At day 0 and day 28, each mouse received a subcutaneously injected vaccination with a volume of 300 μl according to the following scheme:
group a alum-adjuvated PorA vaccine (positive control)
group b non-adjuvated PorA (reference sample)
group c PorA buffer (placebo)
group d 75% non-adjuvated PorA, 25% misfolded PorA (Test item 1)
group e 25% non-adjuvated PorA, 75% misfolded PorA (Test item 2)

Sera were collected at day 7, 14, 21, 28, 35 and 42 and stored at −20° C. The PorA dose was 3 μg for each animal. Vaccines (approximately 6 doses) were prepared as follows:
Group a: 1. 50 μl PorA stock, 2. 200 μl 10× buffer, 3. 50 μl alum stock, 4. 1.7 ml H₂O, 5. 30 minutes at the roller device at room temperature.
Group b: 1. 50 μl PorA stock, 2. 1950 μl 1× PorA buffer.
Group c: 1× PorA buffer.
Group d: 1. 37.5 μl PorA stock, 2. 50 μl heat-denatured (PorA+OVA) in PBS (Misfolding Method I), 3. 50 μl EDC/NHS coupled (PorA+OVA), 307 μg/ml in PBS (Misfolding Method II), 4. 20 μl heat-denatured glutaraldehyde/NaBH₄ coupled (PorA−OVA) in PBS (Misfolding Method III), 5. 1813 μl 1× PorA buffer.
Group e: 1. 12.5 μl PorA stock, 2. 150 μl heat-denatured (PorA+OVA) (Misfolding Method I), 3. 150 μl EDC/NHS coupled (PorA+OVA), 307 μg/ml in PBS (Misfolding Method II), 4. 60 μl heat-denatured glutaraldehyde/NaBH₄ coupled (PorA−OVA) in PBS (Misfolding Method III), 5. 1628 μl×PorA buffer.

### Anti-PorA Antibody ELISA

PorA-specific IgG titers were determined by using standard ELISA setups with each of the three different PorA subtypes, or with the trivalent PorA solution coated in the wells. Briefly, for the ELISA with the three separate PorA subtypes, flat-bottom 96-well microtiter plates (Immulon 2, Nunc, Roskilde, Denmark) were coated overnight at room temperature with outer membrane vesicles (OMVs) comprising one of the three PorA subtypes of Neisseria strains, respectively P1.5-2.10, P1.12-1.13 and P1.7-2.4 (3 μg/ml). Negative control were coated OMVs lacking PorA. After overnight incubation, the plates were washed three times with a 0.03% Tween 80 solution in tap water. The plates were then incubated for 80 minutes at 37° C. with threefold dilutions of the serum samples of each individual mouse, collected at day −3 (pre-immune), 14, 28 (second vaccination) and 42, in PBS containing 0.05% Tween 80. The initial dilution was 100 times. After incubation, the plates were washed three times with 0.03% Tween 80 in tap water. PorA-specific IgG levels were measured by using goat anti-mouse IgG-horseradish peroxidase conjugate (Southern Biotechnology Associated Inc., Birmingham, Ala., USA.) The conjugate was diluted 1:5000 in PBS containing 0.05% Tween 80 and 0.5% skim milk powder (Protifar; Nutricia, Zoetermeer, The Netherlands), and 100 μl was added to the wells. The plates were then washed three times with 0.03% Tween 80 in tap water and once with tap water alone. A peroxidase substrate (100 μl of 3,3′5,5′-tetramethylbenzidine with 0.01% H₂O₂ in 110 mM sodium acetate buffer (pH 5.5) was added to each well, and the plates were incubated for 10 minutes at room temperature. The reaction was stopped by adding 100 μl of 2 M H₂SO₄ to each well. The IgG antibody titers were expressed as the log₁₀ of the serum dilution giving 50% of the maximum optical density at 450 nm. When no signal is obtained with the initially 100-fold diluted serum, the titer was arbitrarily set to 50. The ELISA's are performed at the NVI (Dr G. van den Dobbelsteen).

For the anti-trivalent PorA antibody titer determination, 5 μg/ml of trivalent PorA was coated in the wells of Microlon high-binding plates (Greiner). After blocking with Blocking Reagent (Roche), wells were overlayed with dilution series of sera that were collected at day 21 and that were pooled for each group of mice a-e. Dilution buffer was PBS/0.1% Tween20. Binding of mouse antibodies was detected using 1:3000 RAMPO, and a stain with TMB/H₂SO₄. Absorbance was read at 450 nm.

### Serum Bactericidal Assay

The serum bactericidal assay (SBA) with mouse sera after immunization with PorA of the trivalent strain (1. P1.5-2.10; 2. P1.12-1.13; 3. P1.7-2.4) was performed after two immunizations, with sera collected at day 28 (second vaccination) and day 42, as described above. Briefly, sera were diluted 1:10 in Grey's balanced salt solution containing 0.5% bovine serum albumin and inactivated complement (30 minutes, 56° C.), and serial dilutions were added to 96-well plates. Bacteria are grown in Mueller-Hinton broth (approximately 80 minutes, 37° C.) until the optical density at 620 nm was 0.220 to 0.240, diluted in GBSS containing 0.5% bovine serum albumin, and added to the wells (total concentration, 10⁴ CFU/ml). Each preparation was incubated for 20 minutes at room temperature. Baby rabbit complement (80%) was added, zero-time samples were plated, and the 96-well plates were incubated at 37° C. for 60 minutes. The SBA titer was calculated by determining the log₂ reciprocal of the serum dilution that resulted in ≧90% killing based on the concentration in the zero-time samples. When no signal was obtained with the initial 10-fold diluted serum, the titer was arbitrarily set to 5. SBA titer determinations were performed at the NVI (Dr G. van den Dobbelsteen).

### Statistics

The IgG titer is expressed as the log₁₀value of the geometric mean titer (GMT) obtained for each group of mice plus the standard error of the mean. The SBA titer is expressed as the log₂ average value obtained for each group of mice. Experiments were performed in duplicate. Differences between titers were considered significant at P values of ≦0.05, as determined by the Student t test.

### Results

### Analysis of the Presence of Misfolded PorA with Amyloid-Like Misfolded Protein Conformation Comprising Crossbeta Structure

Trivalent PorA obtained from the NVI was denatured according to three methods: cyclic heat-denaturation in the presence of OVA (Method I), cyclic heat-denaturation of PorA conjugated to OVA using EDC/NHS coupling (Method II) and cyclic heat-denaturation of PorA conjugated to OVA using glutaraldehyde/NaBH₄ coupling (Method III). The presence of amyloid-like misfolded protein conformation was determined using two different assays. The fluorescence enhancement of amyloid-specific dye ThT was assessed (FIG. 6A, C), and the activity enhancement of tPA, which is a serine protease that binds to and is activated by proteins comprising amyloid-like misfolded protein conformation, was determined (FIG. 6B, D). In FIGS. 6A and B it is seen that the PorA stock solutions at 400-fold dilution all comprise crossbeta structure. After formulation of the vaccines a-e, alum-adjuvated control vaccine and 75% crossbeta-adjuvated PorA vaccine test positive for the presence of amyloid-like misfolded protein in the tPA activation assay (FIG. 6D). Based on the ThT fluorescence analysis (FIG. 6C), however, that tested negative for alum-adjuvated PorA, we conclude that the enhanced tPA activation seen with alum-adjuvated PorA is due to in-assay misfolding of tPA/plasminogen at the surface of alum precipitates, resulting in subsequent tPA binding and activation. In conclusion, PorA starting material and misfolded PorA comprise amyloid-like crossbeta structure.

### Anti-PorA Antibody ELISA and SBA

The results of anti-PorA antibody titer determinations using either the trivalent PorA antigen, or the three separate PorA subtypes as the antigen, are depicted in Tables 4-18 and FIGS. 7A and B. Total anti-trivalent PorA antibody titers in pooled sera collected at day 21 are shown in FIG. 7A. It is clear that pooled sera of mouse groups a, b, d, e, that all received vaccine with PorA antigen, have similar total anti-PorA antibody titers, with group c (buffer/placebo) and pre-immune serum testing negative for the presence of anti-PorA antibodies. When titers against each of the three PorA subtypes are assessed, differences between mice within groups are seen with respect to the titer values. The placebo group c tested negative for all mice, as well as the pre-immune serum. Within each of the groups that received PorA vaccine, one or more mice did not elicit titers at all. No differences are seen between groups a, b, d and e.

In FIG. 7C and Tables 19-23, results of the SBA titer determinations with each of the three PorA subtypes are shown. Like with the antigen ELISA, when SBA titers against each of the three PorA subtypes are assessed, differences between mice within groups are seen with respect to the titer values. The placebo group c tested negative for all mice. Within each of the groups that received PorA vaccine, one or more mice did not elicit titers at all. No differences are seen between groups a, b, d and e.

In Table 24, anti-PorA subtype antibody titers and subtype specific SBA titers are compared for each mouse. It is clear that observed titers in the SBA for mouse 3/group a/subtype P1.7-2.4, mouse 1/b/P1.12-1.13, mouse 4/b/P1.7-2.4 and mouse 3/d/P1.5-2.10 are accompanied by the absence of a titer as determined in the antigen ELISA. The opposite, meaning an observed titer in the antigen ELISA with no titer in the SBA, is seen for mice 4/a/P1.7-2.10, 2/b/P1.7-2.10, 2/d/P1.5-2.10, 1/e/P1.5-2.10 and 4/e/P1.7-2.4. This shows clearly one of the difficulties that comes across when developing Neisseria meningitidis subunit vaccines. A positive result in one assay does not have consistent predictive value for the expected result in the second assay. Differences in epitopes for the elicited antibodies and/or affinity/avidity of the elicited antibodies are at the basis of this discrepancy. In addition, it is clearly seen that mice do not elicit relevant antibody titers against all the PorA subtypes. In most cases titers against one or two PorA subtypes are developed. The relative immunogenicity of the three PorA subtypes (P1.5-2.10>>P1.12-2.4≈P1.7-2.4) is reflected in the number of mice that developed titers against the two less immunogenic subtypes, when compared to the most immunogenic subtype P1.5-2.10. When comparing non-adjuvated PorA and conventional alum-adjuvated PorA with crossbeta-PorA adjuvated vaccine, neither differences with respect to numbers of mice that developed antibodies are seen, nor differences are seen with respect to the subtype to which titers are elicited, nor differences are seen with respect to the predictive power of the antigen ELISA, when the accompanied SBA titer is considered. So, in conclusion, in this first test to elicit bactericidal antibodies against PorA it is proven that the Adjuvation-through-crossbeta structure technology provides as potent vaccines as conventionally used alum-adjuvated PorA. The Adjuvation-through-crossbeta structure technology provides several means for improvement of the currently available Neisseria meningitidis multivalent PorA subtype vaccines. For instance, less material is now used to elicit protective titers. More importantly, when less immunogenic subtypes are considered, improvement of effective protective immunogenicity is achieved by using the Adjuvation-through-crossbeta structure technology specifically for those problematic subtypes. After applying the Adjuvation-through-crossbeta structure technology, the subtype with optimal crossbeta structure with respect to potential to elicit protective antibodies, is for instance co-administered in a multivalent subunit vaccine. Alternatively, the technology is applied to a complete multivalent subunit vaccine. Linkage of PorA subtypes with low immunogenic strength to another protein that comprises a potent immunogenic crossbeta structure improves the development of desired antibodies. Furthermore, when applying apart from PorA a protein with crossbeta structure with potent immunogenic strength in a multivalent vaccine, antigen titers with respect to the unrelated protein provide a predictive tool with respect to the presence of bactericidal antibodies.

Finally, today alum is used as the adjuvant of choice in formulations of PorA vaccines. When the Adjuvation-through-crossbeta structure technology is applied in vaccine development, e.g. in PorA vaccine development, it is not necessary anymore to imply an adjuvant other than a proteinaceous molecule comprising crossbeta structure, e.g. alum, for PorA vaccine formulation, in the final formulation. Of course, in other occasions the amount of currently used adjuvants is reduced, rather than completely omitted, which is also beneficial with respect to amongst other things safety issues, cost reduction, ease of use, and stability.

### EXAMPLE 10

### Immunization of Mice with a Human Antigen Comprising Crossbeta Structure Elicits Antibody Titers Against Untreated Human Antigen and Breaks Tolerance in the Mice

Human β2-glycoprotein I, the auto-antigen in the auto-immune disease Anti-Phospholipid Syndrome, with crossbeta structure induces a titer against self-β2-glycoprotein I in mice

### Materials & Methods

### Misfolded β2-Glycoprotein I Immunizations

### Stock Solutions

Stock solution of human β2-Glycoprotein I; 800 μg/ml in 1× Tris-buffered saline, pH 7.2 (1×TBS). Cardiolipin vesicles were prepared from a lamellar solution of cardiolipin (Sigma; C-1649) according to a protocol by Subang et al. (²). Two-hundred μl of cardiolipin was placed into a glass tube and ethanol was evaporated by a constant stream of N₂. The dried cardiolipin was reconstituted in 104 μl of 1×TBS and vortexed thoroughly. The resulting solution contained 10 mg/mL (7.14 mM) of cardiolipin vesicles. This solution could be stored for 14-days at 4° C., maximally. All dilutions were in TBS and after storage, the solution was vortexed before use.

### Modifications: Preparation of alkyl-β2gpi

β2-GPI was reduced and alkylated as follows. Six hundred forty μl of β2-GPI stock was mixed with 640 μl of 8 M Urea (cooled solution) in 0.1 M Tris pH 8.2. The solution was degassed with N₂ gas for approximately 6 minutes. From a 1 M DTT stock 12.8 μl was added to the solution, mixed and incubated for 3 hours at room temperature. A 1 M iodoacetamide (Sigma; I-6125) was prepared, of which 25.6 μl was added to the β2-GPI reaction mixture. The solution was subsequently dialysed against PBS. Misfolding of the resulting alkyl-β2gpi was determined by measuring the enhancement of ThT fluorescence and by the increased ability to activate tPA/plasminogen, resulting in plasmin in the chromogenic assay. The chromogenic assay was performed with 400 pM tPA, 20 μg/ml plasminogen. Signals obtained with alkyl-β2gpi were compared with those obtained with native β2gpi starting material.

### Immunizations of Mice with Native β2gpi, alkyl-β2gpi and Cardiolipin-β2gpi

Female Balb/C AnNHSd (BalB/C, Harlan) 7-9 weeks were housed in filtertop cages in groups of 5 mice per group. After approximately one week of adjustment to the environment, pre-immune sera were drawn. On the start of the first week, mice were given either 100 μl plasma (150 μg/ml) β2-Glycoprotein I, 100 μl alkyl-β2-Glycoprotein I (150 μg/ml) or 100 μl of a mixture of 150 μg/ml of β2-Glycoprotein I with 9.33 μM cardiolipin (CL-β2gpi). This latter sample was prepared by pre-incubating 400 μg/ml of β2-GPI with 25 μM of cardiolipin vesicles for at least 10 minutes at RT after mixing the sample by pipetting; afterwards samples were diluted to 150 μg/ml. The presence of misfolded β2gpi in the CL-β2gpi preparation was determined by measuring enhanced ThT fluorescence and increased potential to stimulate tPA/plasminogen activation. All dilutions were made freshly in TBS and kept on ice. Injections were given intravenously in the tail veins of the mice and given on Mondays and Fridays of the first and third week. Blood was drawn three days prior to the start of the study, and on Wednesdays of week 2 and 4 by puncture of the vena saphena. Blood was collected in Easy collect tubes, with Z serum clot activator. Sera were prepared by centrifugation in a tabletop centrifuge, with a rotor diameter of 7 cm, at 3800 rpm for 10 minutes (slow start and stop) and stored at −20° C. before until further analysis.

### Titer Determinations

Sera were analyzed for antibodies against unmodified native (coated) β2-GPI. Microlon high-binding 96-well plates (Greiner, Alphen aan den Rijn, The Netherlands) were coated with 50 μL native β2-GPI (5 μg/mL in 100 mM NaHCO₃, pH 9.6, 0.05% NaN₃) per well for 1 hour. Then the wells were drained and washed twice with 300 μL phosphate buffered saline (PBS), containing 0.1% Tween20 (PBST). After washing, wells were blocked by incubating with 200 μL Blocking Reagent (Roche, Almere, The Netherlands) in PBS for 1 hour. The wells were drained and washed twice with 300 μL PBST. Antibody titers were determined by adding pooled sera of each experimental group (n=5) in three-fold serial dilutions (starting from 1:30, 50 ul/well) to plates coated with native human β2gpi. The plates were washed four times with 300 μL PBST. Peroxidase-conjugated rabbit-anti-mouse antibodies (RAMPO), diluted 1:3000 in PBST, was added to the wells and incubated for 1 hour. Plates were drained and washed four times with 300 μL PBST and twice with 300 μL PBS. The plates were stained for approximately 5 minutes using 100 μL/well of TMB substrate (Biosource Europe, Nivelles, Belgium), the reaction was stopped with 50 μL/well of 2 M H₂SO₄ and read at 450 nm on a Spectramax340 microplate reader. The absorbance values were plotted against log dilution. Curves were fitted with a sigmoidal curve (GraphPad Prism version 4.02 for Windows, Graphpad Software, Calif., USA). For comparison, the dilution that yielded a residual absorbance after background subtraction of 0.1 was arbitrarily taken as the titer of the various sera.

In a similar ELISA approach, binding of 100-fold diluted sera after immunization with native human β2gpi, alkyl-β2gpi, CL-β2gpi and pre-immune serum to immobilized murine β2gpi was assessed. In this way, it is determined whether immunizations of mice with human β2gpi elicit a humoral auto-immune response against murine β2gpi.

### Results

### Immunization of Mice with Crossbeta-Adjuvated Misfolded β2gpi without the Use of a Conventional Adjuvant

Exposure of human native β2gpi to cardiolipin, or alkylation of cysteine residues in β2gpi induces amyloid-like protein conformation (FIG. 8A, B). Immunization of mice, that received four injections of 15 μg antigen/animal, revealed that alkyl-β2gpi and CL-β2gpi elicited far higher humoral immune responses than native β2gpi (FIG. 8C, D). This shows that crossbeta-adjuvation solely through misfolding of β2gpi accompanied by the appearance of amyloid-like characteristics, renders it with higher immunogenic potential. When antibody titers against mouse self-β2gpi were assessed after immunizations with native human β2gpi, alkyl-β2gpi and CL-β2gpi, it was clearly seen that apart from increased titers of antibodies that bind to human native β2gpi, also auto-immune antibody titers against murine β2gpi were increased when amyloid-like structure is present in human β2gpi (FIG. 8E). This provides further evidence for the insight that amyloid-like properties of proteins are a trigger for immunogenicity, leading to clearance. Crossbeta structure is part of a defense mechanism within the Crossbeta Pathway for clearance of obsolete proteins. Furthermore, it is shown that tolerance is not a decisive aspect for whether a humoral immune response will occur or not. It is the amyloid-like nature of the antigen that determines whether the moiety is considered dangerous to the individual or not, and thus whether exposure of the individual to the amyloid-like moiety should be adequately conquered. Our data show that whether the underlying amino-acid sequence is of self-origin or is of non-self origin is not a primary decisive parameter. New vaccination approaches have become possible with respect to development of vaccines against self-antigens that play a role in diseases other than infections, for example for induction of antibodies to LHRH for immunocastration of boars, or for use in preventing graft versus host (GvH) and/or transplant rejections.

### EXAMPLES 11-14

### Materials & Methods for Immunization Trials with E2, CL3, H5, H7

### Cloning, Expression and Purification of Antigens

### Avian Influenza Haemagglutinin-5

Haemagglutinin-5 (H5 or HA5) cDNA of virus strain A/Vietnam/1203/2004 was a kind gift of Dr. L. Cornelissen (ID-Lelystad, The Netherlands). DNA was amplified using primers CAI 127 and 129 and was supplied in a plasmid. At the ABC-expression facility (R. Romijn and W. Hemrika, University of Utrecht, The Netherlands), H5 cDNA was further amplified using primers 5′ ggatcc gatcagatttgcattggttacc 3′ and 5′ gcggcegccagtatttggtaagttcccat 3′. The PCR fragment was ligated in pCR4-TOPO vector. Sequence analysis was performed at Baseclear (Leiden, The Netherlands). The H5 sequence of clone 709-5 contained two silent mutations (See Sequence ID 1; bold/underlined, a→g and t→a). The H5 DNA fragment was digested BamHI and NotI, purified and ligated into pABC-CMA-dE-dH-sub-optimal_sp-Flag3C-his C_(pUC) (See Sequence ID 2; sub-optimal signal sequence underlined/Italics, FLAG-tag-His-tag bold/underlined) and pABC-CMV-dE-dH-Cystatine_sp-Flag3C-his C_(pUC) expression vectors (ABC-expression facility). In this way, H5 is expressed with a carboxy-terminal FLAG-tag-His-tag.

### Expression and Purification of H5

For expression of H5-FLAG-His protein, a 2-liter culture of HEK293E (human embryonic kidney) suspension cells were transiently transfected with expression vector using polyethylene-imine. Transfected cells were grown for 5-6 days. For purification of H5 secreted in the cell culture medium the cells were pelleted and the supernatant was concentrated using a Quixstand concentrator (A/G Technology corp.), using a 10 kDa cut-off filter (GE Healthcare). A dialysis step was performed on the same concentrator, and the proteins were dialysed against PBS/1M NaCl. The concentrated and dialysed medium was filtered (0.45 μm, Millipore) and incubated with Ni-Sepharose Fast Flow beads (GE-Healthcare 17-5318-02) in the presence of 7.5 mM imidazole, for 3 h at room temperature under constant motion. A column was filled with the beads and the proteins were extracted by increasing imidazole concentration. The purification was performed on an AKTA Explorer (Pharmacia). Fractions with H5 were pooled and again loaded on the Ni-Sepharose beads for further purification. Fractions with H5 were pooled and dialyzed against PBS and subsequently H5 concentration was determined using a standard BCA Protein Assay Reagent kit (Pierce No. 23225). The molecular weight of H5 is approximately 75 kDa. Pooled H5 solution (236 μg/ml) was aliquoted and stored −80° C. (lot 1 CS210406). A second batch of H5 protein (lot 2 fraction X 250506CS) had a concentration of 140 μg/ml in PBS.

### Avian Influenza H7

H7 cDNA of virus strain A/Chicken/Netherlands/621557/03 was a kind gift of Dr. L. Cornelissen (ID-Lelystad, The Netherlands, ‘BglII-NotI, amplified using primers RDSH7′5 and RDS7′3), and was supplied as a PCR fragment. At the ABC-expression facility (R. Romijn and W. Hemrika, University of Utrecht, The Netherlands), the H7 PCR fragment was amplified using primers 5′ agatct gacaaA(g)atctgccttgggcatcat 3′ and 5′ gcggccgcaagtatcacatctttgtagcc 3′. The PCR fragment was ligated in pCR4-TOPO vector. Sequence analysis was performed at Baseclear. The H7 sequence of clone 710-15 contained four mutations, of which two result in amino-acid mutations (See Sequence ID 3 and 4; a→g, g→a, g→a (Arg→Lys), a→g (Met→Val)). The H7 DNA fragment was isolated upon BamHI and NotI digestion, and ligated into pABC-CMV-dE-dH-sub-optimal_sp-Flag3C-his C_(pUC) (See Sequence ID 4; sub-optimal signal sequence underlined/Italics, FLAG-tag-His-tag bold/underlined) and pABC-CMV-dE-dH-Cystatine_sp-Flag3C-his C_(UC) expression vectors (ABC-expression facility). In this way, H7 is expressed with a carboxy-terminal FLAG-tag—His-tag.

### Expression and Purification of H7

The cells from a 2 liter cell suspension were pelleted by centrifugation and the cell pellet was isolated and resuspended in 25 mM Tris, 0.5 M NaCl, pH 8.2 to a volume of 50 ml. The cells were freeze-thawed once and five Protease Inhibitor Cocktail Tablets (Roche Cat. No. 11 836 170 001) were added to the cell suspension. The cell suspension was sonicated on ice and centrifuged at 21,000*g for 1 h at 4° C. The supernatant was filtered (0.45 μm) and incubated for 16 h at 4° C. under constant motion, with Ni-Sepharose Fast Flow beads in the presence of 20 mM imidazole. After filling a column H7 was eluted by increasing imidazole concentration. Expression of H7 was analyzed on polyacryl-amide gel using Coomassie and on a Western blot using 1:3000 anti-FLAG-HRP (Sigma A-8592). Fractions with H7 were pooled and dialysed against PBS pH 7.4 at 4° C., aliquoted and stored at −80° C. The concentration of the pooled H7 solution was determined by densitometry on a Western blot using purified E2-Flag with known concentration for the standard curve, and was 10.5 μg/ml (molecular weight of H7 is approximately 75 kDa (lot 2 05-2006CS).

### Expression by HEK293E Cells and Subsequent Purification of E2

DNA of the glycoprotein E2 of Classical Swine Fever virus (CSFV) was obtained from Geneart (Regensburg, Germany, Sequence ID 5 and 6). The construct was digested using BamHI and NotI, and ligated into vector pABC674 (ABC-expression facility), which will extend the recombinant E2 with a carboxy-terminal FLAG-tag-His-tag. HEK293E cells were transiently transfected and grown for 5-6 days (C. Seinen, University Medical Center Utrecht, The Netherlands). Purification was essentially similar to the method described for H5 and H7. Binding buffer for the Ni²⁺-column was 25 mM Tris, 0.5 M NaCl, pH 8.2. After dialysis of pooled fractions with E2, purity was determined from gel using ImageQuant software (Molecular Dynamics). E2 purity was approximately 90%. Protein concentration was measured with the BCA method, and was 655 μg/ml in PBS. The concentration E2 was 561 μg/ml in the aliquoted stock solution that was stored at −80° C. (lot 1 210406CS).

### Expression by Sf21 Cells and Subsequent Purification of E2

Recombinant glycoprotein E2 of CSFV was also expressed and purified for testing a new crossbeta-adjuvated E2 vaccine against CSFV. The production of this E2 was performed by R. D. Strangi (TU Eindhoven, The Netherlands) at the Animal Sciences Group (ASG, ID-Lelystad, The Netherlands). An aliquot of Spodoptera frugiperda (Sf21) cell line (P. A. van Rijn, ID-Lelystad, ⁴) from liquid nitrogen was rapidly thawed to 37° C. in a water bath. Then, 1.5 ml cell culture was transferred to a 150-cm² flask with 27 ml prewarmed grow medium (SF900-II serum free medium with L-Glutamine, Gibco) supplemented with 1% v/v of antibiotic-antimycotic (Gibco, 15240-062), and grown at 28° C. Sf21 cultures were subsequently expanded in separate 150-cm² flasks up till 30 ml working volume of cell suspension.

Sf21 cells grown in SF900-II medium were infected with E2-expressing baculovirus as described by Hulst et al. (⁵) at an multiple of infection (M.O.I.) of 0.01 and incubated for 280 hr at 28° C. On several time points, the E2 expression level in medium was determined by surface plasmon resonance (SPR), as described below. Culture medium was cleared from cell debris by centrifugation for 10 minutes at 600×g, and stored at 4° C. after adding NaN₃ to a final concentration of 0.02%.

### Surface Plasmon Resonance with Anti-E2 Antibody

Binding experiments with anti-E2 antibody and cell culture medium comprising recombinant E2 were performed using SPR on a Biacore 3000 instrument (Biacore AB, Uppsala, Sweden) using a CM5 research grade chip. A standardized amine coupling procedure was used to covalently couple proteins to the sensor surface, implying first activation of the dextran surface of the CM5 sensor chip with a 7-minutes injection of a 1:1 mixture of 100 mM N-hydroxysuccinimide (NHS) and 400 mM N-ethyl-N′-(dimethyl-aminopropyl)-carbodiimide (EDC) with a flow rate of 5 μl/minute. Anti-E2 antibody α-V3 (ID-Lelystad) was diluted 1:40 in acetate buffer pH 5.5 and covalently coupled to the activated dextran by a 7-minute injection at a flow rate of 10 μl/minute. Remaining activated groups on each flow cell were blocked by injection of 35 μl of 1 M ethanolamine hydrochloride pH 8.5. Dissociation was initiated upon replacement of the injected sample by running buffer. Residual response units (RU's) after 2 minutes of dissociation were determined. Filtered and degassed HBS-EP buffer (150 mM NaCl, 2 mM EDTA, 0.005% (v/v) Tween-20, and 10 mM HEPES, pH 7.4) was used as running buffer (Biacore). Culture medium was diluted 1:10 in running buffer and binding of E2 to immobilized α-V3 was determined (not shown).

### α-V3 Anti-E2 Antibody Affinity Column

Monoclonal anti-E2 antibody α-V3 (9 mg/ml, produced and purified at ASG, ID-Lelystad) was dialyzed against 0.1 M NaHCO₃ with 0.5 M NaCl, pH 8.3. Dialysis membrane (Medicell International Ltd, with a molecular weight cut-off of 12-14,000 Da) was heated for 30 minutes in water with 2% (w/v) sodium bicarbonate and 1 mM EDTA pH 8.0. Subsequently, it was boiled for 10 minutes in 1 mM EDTA pH 8.0. Antibody α-V3 was dialyzed against 4 liter of 0.1 M NaHCO₃ with 0.5 M NaCl pH 8.3 at 4° C., which was refreshed four times in total. Dialyzed monoclonal antibody α-V3 as coupled to CNBr-activated Sepharose-4B (Amersham Biosciences Aβ, Uppsala Sweden) according to the manufacturer's instructions. One and a half gr CNBr-activated Sepharose 4B was swollen in 300 ml of 1 mM HCl for 15 minutes. Then the Sepharose beads were washed four times by spinning it down shortly and replacing the supernatant by fresh 1 mM HCl. α-V3 antibody in 0.1 M NaHCO₃ with 0.5 M NaCl, pH 8.3 was added and was incubated for 2 hr at room temperature on a roller device. Unbound α-V3 was washed away with 50 ml of 0.1 M NaHCO₃ with 0.5 M NaCl, pH 8.3. Subsequently any remaining active groups at the matrix were blocked by a 1 hr incubation with 20 ml of glycine pH 8.0. Then three washes with alternating pH (0.1 M NaHCO₃ with 0.5 M NaCl, pH 8.3 followed by 0.1 M Na-acetate with 0.5 M NaCl pH 4.0) was performed. Finally, a wash with 10 ml of 0.1 M Glycine pH 2.5 was performed, followed by a wash with PBS. Then the column was stored in PBS supplemented with 0.02% w/w NaN₃, at 4° C.

### E2 Purification

After 280 hr from the start of the virus infection of the expanded Sf21 cells, the culture medium was collected in 50 ml tubes and cleared from cells and cell debris by centrifugation for 10 minutes at 600×g, and stored at 4° C. Supernatant was separated from the cell pellet and 0.02% w/w azide was added. E2 was purified from cell culture supernatant in three subsequent runs, as described below. Run 1 (lot1 200406RS): E2 culture medium was circulated over the α-V3 affinity column at room temperature for 2.5 hr at a flow rate of 60 ml/hr, followed by an overnight wash with PBS. E2 was eluted with 0.1 M glycine pH 2.5. Eluate was collected in 3 ml fractions and directly adjusted to pH 7-8 by addition of 45 μl of 3 M Tris-base. Subsequently, the fractions containing purified E2 protein were dialyzed against PBS, and analyzed by SDS-PAGE. After protein quantification using the BCA Protein Assay (Pierce), fractions with >95% pure E2 were pooled, and stored at −80° C. E2 concentration in the pooled fraction was 285 μg/ml for lot 1 200406RS. An additional amount of E2 was extracted from the same cell culture supernatant by recirculating over the α-V3 affinity matrix (Run 2, lot2 030506RS). Run 3 (lot3 100506RS): A second batch of cell culture medium with expressed E2 was first stored at −80° C. and then used for E2 purification as described above.

First, medium was concentrated using 15 ml Macrosep 10K concentrators (Pall). The 3.3 times concentrated E2 medium was circulated over the affinity column at room temperature for 5 hr. at a flow rate of 60 mL/hr, followed by 5 hr wash with PBS. After the first purification step, the concentrated medium was reloaded on the column for a subsequent purification. E2 was analyzed by SDS-PAGE and immunoblotting. Samples were applied onto a 4-15% poly-acrylamide-gel (SDS-PAGE, NUPAGE, Invitrogen). Prior to SDS-PAGE, the samples were heated for 5 min at 95° C. in the presence of 20 mM DTT. Separated proteins were transferred to nitrocellulose blot membrane and E2 protein was visualized by an incubation with first α-V3 and then RAMPO, followed by staining with chemiluminescence (Western Lightning, Perkin-Elmer).

### Expression and Purification of Fasciola Hepatica Caihepsin L3 Protein

Recombinant DNA of the cathepsin L3 protein of Fasciola hepatica (CL3 protein) was obtained from Geneart (See Sequence ID 7). The construct was digested using BamHI and NotI, and ligated into vector pABC674 (ABC-expression facility), which will extend the recombinant CL3 protein with a carboxy-terminal FLAG-tag-His-tag. After expression for 5-6 days, pelleted HEK293E cells from a 2-liter suspension culture were resuspended in 25 mM Tris, 0.5 M NaCl, pH 8.2 to a volume of 50 ml. The cells were freeze-thawed once and five Protease Inhibitor Cocktail Tablets (Roche Cat. No. 11 836 170 001) were added to the cell suspension. The cell suspension was sonicated on ice and centrifuged at 21,000*g for 1 h at 4° C. The supernatant was filtered (0.45 μm, Millipore) and imidazole was added to a final concentration of 10 mM. The CL3 protein was loaded/re-loaded for 16 h at 4° C. on a HisTrap HP 1 ml column (GE Healthcare 17-5247-01) with a flow rate of 0.75 ml/minute, using a closed system. Bound CL3-FLAG-His was eluted upon applying an imidazole gradient to the column. The purified protein was visualized by SDS-PAGE electrophoresis (Invitrogen, NuPage 4-12% BisTris NP0323) with Coomassie stain (Fermentas PageBlue R0571). In addition, CL3 was subjected to Western blotting followed with a stain using anti-FLAG-HRP (Sigma A-8592) and luminol-based substrate for HRP-catalyzed detection (Western Lightning Chemi-luminescence Reagent Plus Cat. No. NEL 104). Purity of pooled fractions with CL3 protein, dialyzed against PBS, was estimated with densitometry on Coomassie stained gel, and was approximately 7.5%. Total protein concentration was determined by measuring absorbance at 280 nm and calculating using the assumption that a solution with 1 mg/ml protein will result in an absorbance value of 1.0. Total protein concentration was 400 μg/ml. Taking the purity into account, the CL3 protein concentration is approximately 30 μg/ml in PBS. The molecular weight of CL3 protein is approximately 38 kDa including one N-linked carbohydrate. Protein solution was aliquoted and stored at −80° C. (lot 1 010606CS).

### Fasciola Hepatica Cathepsin L3 Peptide Conjugation to Key-Hole Limpet Haemocyanin

For immunization trials Cathepsin L3 peptide of Fasciola hepatica (CL3 peptide, Ansynth Service B. V, Roosendaal, The Netherlands, Lot BB1; sequence taken from Cathepsin L-like cysteine proteinase UniProtKB/Swiss-Prot entry www.expasy.org/uniprot/P80528, and extended with an amino-terminal cysteine: CSNDVSWHEWKRMYNKEYNG; Sequence ID 8) was used. The amino-terminal cysteine was introduced for coupling purposes to Imject maleimide activated mariculture Keyhole Limpet Haemocyanin (mcKLH) carrier protein (Pierce). Conjugation of CL3 peptide to maleimide-activated mcKLH was performed according to the manufacturers manual. Lyophilized CL3 peptide was dissolved in supplied conjugation buffer (containing 83 mM sodium phosphate buffer, 0.1 M EDTA, 0.9 M NaCl, 0.02% sodium azide, pH 7.2) to a final concentration of 10 mg/ml. Maleimide-activated mcKLH was reconstituted in distilled water to 10 mg/ml. The two solutions were mixed and incubated for 2.5 hrs at room temperature on a roller device. After conjugation, the formed precipitates were separated from the supernatant by centrifugation for 10 minutes at 16.000×g. The pellet with precipitates was stored on ice. The conjugate in solution was purified from excess free CL3 peptide by applying the supernatant to D-Salt Dextran Desalting Columns (molecular weight cut-off 5 kDa, Pierce) with running buffer comprising 83 mM sodium phosphate, 0.9 M NaCl pH 7.2. Fractions of 0.5 ml were collected and protein concentration was determined with the BCA method (Pierce). Presence of conjugate was assessed with SDS-PAGE/Coomassie and with an ELISA. The fractions with conjugate were pooled and the isolated pellet was subsequently dissolved in the pooled fractions. Then, CL3-KLH conjugate was dialyzed against 4 liter PBS. Protein quantification was performed using the BCA protein assay (Pierce) and the conjugate suspension was stored at 4° C. The CL3-KLH concentration was 1.35 mg/ml. For the ELISA, concentration series of free CL3 peptide, free KLH and CL3-KLH conjugate, as well as coat buffer only were coated onto wells of a Microlon high-binding 96-wells plate (Greiner). After blocking with Blocking reagent (Roche) wells were overlayed with 500-fold diluted mouse anti-CL3 serum (ID-Lelystad, supplied by A. Antonis, code ‘YM30, III C11C7E8’) in PBS/0.1% v/v Tween20. Binding of anti-CL3 antibody was visualized using RAMPO (DAKOCytomation, P0260, lot 00020228)-1,2-phenylenediamine/H₂SO₄-absorbance reading at 490 nm. The CL3 fraction in the CL3-KLH conjugate was estimated by comparing antibody binding with that obtained with the coated free CL3 peptide standard. The CL3 (:) KLH ratio was approximately 1(:)1.

### Vaccine Preparation: Formation of Amyloid-Like Misfolded Protein Conformation Comprising Crossbeta Structure

Introduction of amyloid-like misfolded protein conformation in the various antigens is achieved using different misfolding techniques. The extent of misfolding was assessed by analyzing the ability of an antigen solution to enhance ThT fluorescence and/or by assessing the ability to stimulate tPA-mediated conversion of plasminogen to plasmin.

### Misfolding Method I with H5: Cyclic Thermal Misfolding of H5 Mixed with OVA

H5 stock used for misfolding purposes: 236 μg/ml H5 in PBS lot 1 CS210406. OVA was dissolved in H5 solution to a final concentration of 1 mg/ml. Subsequently, the H5/OVA solutions was subjected to cyclic thermal misfolding following the procedure described above for PorA.

### Misfolding Method II with H5 from HEK293E Cells: Misfolding by Thermal Cycling with H5 Conjugated with Ovalbumin, Using EDC-NHS Coupling

The H5 stock used was stock: 236 μg/ml native H5 in PBS lot 1 CS210406. Similar to PorA (see above), H5 obtained from HEK293E cells was conjugated with OVA. H5 concentration and OVA concentrations were 169 μg/ml and 1000 μg/ml, respectively. After coupling, solutions were dialyzed against PBS. The solutions were subsequently transferred to PCR cups and conjugates were misfolded upon cyclic thermal denaturation.

### Misfolding Method III Applied to H5: Coupling of Polypeptide-A to Polypeptide-B by Glutaraldehyde/NaBH₄ Activation

Similarly to PorA, for coupling of H5 to OVA, both proteins were activated with glutaraldehyde and sodium-borohydride and mixed. For this purpose 250 μg OVA was dissolved in 1 ml H5 stock solution and 180 μl PBS was added. Glutaraldehyde (25% (v/v) solution in H₂O, Merck, Hohenbrunn, Germany, 8.20603.1000 (UN2927, toxic), lot S4503603 549), pre-diluted to a 4% 100× stock in H₂O was added to a final concentration of 0.04%. After vortexing and a 2-minutes incubation at room temperature, a 120 mM 100× stock NaBH₄ (approx. 98%, Sigma, St. Louis, Mo., USA, S9125, lot 53H3475) was added to a final concentration of 1.2 mM. The solution was vortexed and incubated for 42 h at room temperature at a roller device. Then, the solution was extensively dialyzed against PBS. The conjugate solution was subsequently heated for five thermal cycles as described above.

### Misfolding Method I with H7: Cyclic Thermal Misfolding of Free H7 or H7 Mixed with OVA

H7 stock used for misfolding purposes: 21.4 μg/ml H7 in PBS lot 1 CS210406. H7 solution was either subjected to thermal misfolding without any addition, or H7 was thermally misfolded after dissolving OVA to a final concentration of 1 mg/ml in the H7 stock solution. Cyclic thermal misfolding was performed as described for PorA, above.

### Misfolding Method II with H7: Misfolding by Thermal Cycling with H7 Conjugated with Ovalbumin, Using EDC-NHS Coupling

H7 stock used for misfolding purposes: 21.4 μg/ml H7 in PBS lot 1 CS210406. Similar to PorA (see above), H7 expressed by HEK293E cells was conjugated with OVA. The final H7 concentration was 10.7 μg/ml, the OVA concentration was 1 mg/ml. After coupling, the conjugate solution was dialyzed against PBS. The solution was subsequently transferred to PCR cups and the conjugate was misfolded upon cyclic thermal denaturation, by applying one cycle from 30° C. to 85° C. at 5° C./minute, and quickly to 4° C.

### Cyclic Thermal Misfolding of E2 from Sf1 Cells

Purified E2 expressed by Sf1 cells in PBS was heated for five cycles in PCR cups in a PTC-200 thermal cycler (MJ Research, Inc., Waltham, Mass., USA). In each cycle, protein was heated from 30 to 85° C. at a rate of 5° C./min, and quickly cooled back to 30° C. before a new cycle started. Finally, heat-denatured E2 was kept at 4° C. Final E2 concentration was 280 μg/ml (lot2 030506RS).

### Misfolding Method IV: Reduction-Alkylation of Cys Residues in E2 from Sf1 Cells

Alkylated E2 is obtained by reducing disulphide bonds, followed by alkylating of the formed free Cys residues. First, urea was added to a final concentration of 8 M, to 353 μg/ml E2 and it was mixed by gentile swirling. Then dithiothreitol (DTT) was added to a final concentration of 10 mM. Air in the tube was replaced by nitrogen gas to inhibit possible oxidation of the reduced cysteines. Then it was incubated for 2 hrs at room temperature on a roller device. Subsequently the solution was chilled (on ice) and iodoacetamide (Sigma) was added to a final concentration of 20 mM. Finally, the alkylated E2 was dialyzed against 1 L of PBS for 4 hrs, followed by 7 hrs against 5 L and 9 hrs against 5 L of PBS. The concentration of alkyl-E2 after dialysis was determined using the BCA protein assay (Pierce), and was 167 μg/ml.

### Misfolding Method I with E2 and OVA: Misfolding by Thermal Cycling

One mg of OVA was dissolved in 1 ml of E2 solution in PBS (OVA concentration is 1 mg/ml, E2 concentration is 280 μg/ml; lot2 030506RS). Solutions were subjected to cyclic thermal misfolding by heating from 30° C. to 85° C. in intervals of 5° C./minute, and back cooling to 30° C. before start of the next cycle (5 cycles). Enhancement of ThT fluorescence and enhancement of tPA/plasminogen activity was assessed.

### Misfolding Method II with E2 from Sf1 Cells: Misfolding by Thermal Cycling with E2 Conjugated with Ovalbumin or KLH, Using EDC-NHS Coupling

Similar to PorA (see above), E2 obtained from Sf1 cells was conjugated with either OVA, or KLH. E2 concentration and OVA concentrations were 193 μg/ml and 1047 μg/ml, respectively. E2 and KLH concentrations were 145 and 631 μg/ml, respectively. After coupling, solutions were dialyzed against PBS. The solutions were subsequently transferred to PCR cups and conjugates were misfolded upon cyclic thermal denaturation.

### Misfolding Method II with E2 from 293E Cells: Misfolding by Thermal Cycling with Free E2

As described for PorA, E2 purified from HEK293E cell culture supernatant (lot 1 210406CS, 561 μg/ml in PBS) was subjected to thermal misfolding using a PCR apparatus.

Misfolding Method II with E2 from 293E Cells: Misfolding by Thermal Cycling with E2 Conjugated with Ovalbumin or KLH, Using EDC-NHS Coupling

Similar to PorA and to E2 from Sf1 cells (see above), recombinant E2 obtained from HEK293E cells was conjugated with OVA. E2 concentration and OVA concentrations were 561 μg/ml and 1 mg/ml, respectively. After coupling, solutions were dialyzed against PBS. The solutions were subsequently transferred to PCR cups and conjugates were misfolded upon cyclic thermal denaturation. (lot 1 210406CS, 561 μg/ml in PBS)

### Misfolding Method V: Misfolding of Free CL3 Peptide: Thermal Misfolding

The free CL3 peptide was dissolved at 1 mg/ml in H₂O and used directly for preparation of vaccines, or kept at 65° C. or 37° C. for several days before use in vaccines.

### Misfolding Method I with CL3 Peptide and OVA: Misfolding by Thermal Cycling

One mg of OVA and 1 mg of CL3 peptide, or 1 mg of lyophilized KLH and 1 mg of CL3 peptide were mixed in two separate cups and dissolved in 1 ml PBS (all protein/peptide concentrations are 1 mg/ml). Solutions were subjected to cyclic thermal misfolding by heating from 30° C. to 85° C. in intervals of 5° C./minute, and back cooling to 30° C. before start of the next cycle (5 cycles). As a positive control, 1 mg and 10 mg/ml OVA were also subjected to cyclic thermal misfolding. Enhancement of ThT fluorescence and enhancement of tPA/plasminogen activity was assessed.

### Misfolding Method II with CL3 Peptide: Misfolding by Thermal Cycling with CL3 Conjugated with Ovalbumin or KLH, Using EDC-NHS Coupling

Similar to PorA (see above), CL3 peptide was conjugated with either OVA, or KLH. Both CL3 peptide concentration and OVA or KLH concentration was 1.28 mg/ml. After coupling, solutions were dialyzed against PBS. The solutions were subsequently transferred to PCR cups and conjugates were misfolded upon cyclic thermal denaturation.

### Misfolding Method I with CL3 Protein and OVA: Misfolding by Thermal Cycling

One mg of OVA was dissolved in 1 ml of 30 μg/ml CL3 protein in PBS. The solutions was subjected to cyclic thermal misfolding by heating from 30° C. to 85° C. in intervals of 5° C./minute, and cooling back to 30° C. before start of the next cycle (5 cycles). In addition, the 30 μg/ml CL3 protein stock in PBS was misfolded upon thermal cycling without addition of protein. CL3 protein stock used was lot 1 010606CS (see above).

### Results

### Introduction of Amyloid-Like Structure in Antigens

### Ovalbumin

Lyophilized OVA was dissolved carefully allowing it to fold correctly in a native state, with as little amyloid-like properties as possible. Despite these efforts, OVA displays characteristics that are a hallmark for the presence of at least a fraction of the molecules with crossbeta structure, as shown by the interaction with amyloid-specific dye ThT and by the enhanced activation of tPA and plasminogen (FIG. 9A, B). After cyclic thermal misfolding, however, signals representative for crossbeta structure are far more pronounced with denatured OVA (DOVA, FIG. 9A, B). The data shown are representative measures for routinely prepared amyloid-like misfolded DOVA and OVA with a more native fold. The large differences in crossbeta content make the OVA/DOVA couple interesting items for vaccination purposes. Addition of DOVA to a native antigen or to a partly amyloid-like misfolded antigen serves as a potent crossbeta structure adjuvant.

### H5 for Cocktail Vaccine Preparation

Recombinant H5 with a carboxy-terminal FLAG-tag-His-tag was expressed and purified in-house. Determination of ThT fluorescence enhancing properties and tPA/plasminogen activation properties revealed that the untreated purified H5 already comprises some amyloid-like misfolded protein (FIG. 9C, D, E). In H5 lot 1 210406CS some tPA activating moieties are detected. With H5 lot 2 fraction X 250506CS assessing tPA/plasminogen activating properties was hampered due to the presence of plasmin substrate converting activity in the H5 solution, when tPA was omitted from the reaction mixture. Upon thermal cycling of H5 mixed with OVA or H5 conjugated with OVA using EDC/NHS, ThT fluorescence is strongly enhanced, showing an increase in crossbeta structure content in the H5 antigen solution, compared to untreated H5 (FIG. 9E). The H5 preparations were not only used for preparing the mouse cocktail vaccine with E2, CL3 and H7, but were also used for monovalent H5 immunization of mice.

### H7 Used for Cocktail Vaccine Preparation

Recombinant H7 of strain A/Netherlands/219/03 (Protein Sciences Corp.) was used as a source of untreated antigen for preparation of a cocktail vaccine together with E2, H5, CL3, OVA. However, we detected some ThT fluorescence enhancing capacity with the untreated H7, and also activation of tPA/plasminogen was enhanced by introducing the H7 in the reaction mixture (FIG. 9F, G). These results show the presence of at least a small fraction of H7 molecules with crossbeta structure. In FIGS. 9H and I, ThT fluorescence enhancement with in-house produced recombinant native H7, thermal misfolded mixture of H7 and OVA, and thermal misfolded H7−OVA conjugate, obtained through EDC/NHS coupling is shown, as well as the influence on tPA activity. For the ThT assay, H7 stock solutions were diluted tenfold. In the tPA/plasminogen activity assay H7 was used at 1 μg/ml. The assays show an increase in crossbeta structure content upon mixing or conjugation to OVA, followed by thermal cycling.

### E2 Expressed in HEK293E Cells for Use in a Cocktail Vaccine

Recombinant E2 protein of CSFV was expressed in-house in HEK293E cells and purified from cell-culture supernatant, and used in mouse immunization trials. Purified E2 was subjected to two methods of misfolding: thermal cycling between 30 and 85° C. with the free E2, or thermal cycling after conjugating E2 with OVA, using EDC/NHS coupling. In FIG. 9J, an increase in ThT fluorescence is clearly seen upon using the misfolding methods. Influence on tPA activity could not be assessed due to substrate converting activity in the purified E2 solution, indicative for the presence of trace amounts of plasmin-like protease. Differences seen between untreated E2 and misfolded E2, in potency to enhance ThT fluorescence clearly show the increase in crossbeta structure content upon applying the misfolding procedures.

### CL3 Peptide Used for Incorporation in a Cocktail Vaccine

The CL3 fragment of 19 amino-acid residues and an amino-terminal Cys extension for coupling purposes was subjected to various protein-protein conjugation methods and subsequently to protein misfolding methods. In FIGS. 9K and L it is shown that all CL3 peptide preparations comprise crossbeta structure conformation to some extent, when concerning the property to enhance ThT fluorescence and to further stimulate tPA/plasminogen. Even the free peptide comprises crossbeta structure after dissolving in H₂O.

In a tPA/plasminogen activation assay, freshly dissolved CL3 peptide and 65° C.-incubated peptide are most potent tPA activators, whereas incubations at room temperature or at 37° C. result in a lower activating crossbeta structure content (FIG. 10D). Peptide concentration in the assay was 200 μg/ml. Peptides were incubated in the dark for several days at the indicated temperatures.

### Crossbeta-Antigen H5 for H5N1 Virus Challenges

The stock solution of untreated H5 for preparation of misfolded H5 and for use in vaccine preparations was the 236 μg/ml recombinant H5 stock in PBS (lot 1 210406CS, strain A/Vietnam/1203/2004) for the first vaccination and the 140 μg/ml H5 in 25 mM Tris pH 8.2, 500 mM NaCl (lot 2 fraction X 240506CS) solution for the second vaccination. H5 with amyloid-like misfolded protein conformation was with both H5 lots obtained by applying Misfolding Methods I-III (see above). In FIG. 10A, enhancement of ThT fluorescence is shown when 24 μg/ml H5 of each of the four stock solutions is tested. It is clear that the modifications introduce a significant increase in crossbeta structure content in all three misfolded H5 preparations, when compared to untreated H5.

### Crossbeta-Antigen E2 for CSFV Challenges

For a first vaccination against CSFV, untreated E2, cyclic thermal misfolded E2 (Method I) and alkyl-E2 (Method IV) were used (lot1 200406RS, 285 μg/ml in PBS). The presence of crossbeta structure in alkyl-E2 (Misfolding Method IV) and cyclic thermal misfolded E2 (Misfolding Method I) is shown by the strongly enhanced ThT fluorescence and the increase in tPA/plasminogen activation (FIG. 10B, C).

For the second immunization recombinant E2 expressed by Sf1 cells was again used, now from lot 2 030506RS, 280 μg/ml in PBS. E2 was misfolded using four Misfolding Methods: cyclic thermal denaturation of free E2 (Method I), of E2 in the presence of OVA (Method I), of E2-OVA conjugate obtained by EDC/NHS coupling (Method II) and of E2-KLH conjugate also obtained by EDC/NHS coupling (Method II). These misfolded crossbeta-E2 preparations were mixed 1:1:1:1 before incorporation in vaccine formulations.

### Example 11

### Immunization of Mice with a Vaccine Cocktail Comprising Classical Swine Fever Antigen E2, Fasciola Hepatica Antigen Cathepsin L3 Peptide and Protein, Avian Flu Antigen Haemagglutinin 5 and Avian Flu Antigen Haemagglutinin 7, Together with Ovalbumin

### Dose Response Study and a Test for the Immunogenicity of Crossbeta Structure-Adjuvated Antigen

### Aim

Determination if Classical Swine Fever antigen E2, Fasciola hepatica antigen Cathepsin L3 peptide and protein, Avian flu antigen haemagglutinin 5 and Avian flu antigen haemagglutinin 7, together with OVA antigen with amyloid-like misfolded protein conformation, combined in a cocktail vaccine, elicit antibody titers without the further use of an adjuvant: Adjuvation through crossbeta structure, i.e. with proteins comprising amyloid properties, as defined herein. Therefore, mouse sera were analyzed at day 28 post-immunization for antibody titers against individual antigens.

### Antigen Stock Solutions

Protein Solutions Used for Vaccination
I. Ovalbumin (chicken egg albumin, OVA, Sigma; catalogue number A5503) at 1 mg/ml in PBS was freshly prepared (dissolved by pipetting; 30 minutes at a roller device at room temperature; 1 h at 37° C.; 1 h roller device at room temperature; 4° C. storage)
II. Amyloid-like misfolded OVA (DOVA) at 1 mg/ml in PBS was obtained according to the heat denaturation protocol as described above
III. Cathepsin L3 peptide (Ansynth Service B. V., Roosendaal, Netherlands, Lot BB1; sequence CSNDVSWHEWKRMYMKEYNG (CL3 peptide with amino-terminal Cys extension)) was dissolved at 1 mg/ml in PBS and kept at 4° C.
IV. Amyloid-like misfolded CL3 peptide-KLH conjugate was obtained upon heat-denaturation (conjugate concentration is 1.35 mg/ml in PBS; approximately 50% CL3 peptide) lot 1 05-2006RS (see above)
V. CL3 protein (30 μg/ml in PBS) lot 1 010606CS (see above)

VI. Amyloid-like heat-denatured CL3 protein (30 μg/ml in PBS) lot 1 010606CS
VII. Amyloid-like misfolded CL3 protein-OVA conjugate, heat-denatured (30 μg/ml in PBS) lot 1 010606CS
VIII. E2 (561 μg/ml in PBS) lot 1 210406CS (see above)
IX. Amyloid-like misfolded E2 heat-denatured (561 μg/ml in PBS) lot 1 210406CS
X. Amyloid-like misfolded E2-OVA conjugate, heat-denatured (561 μg/ml in PBS) lot 1 210406CS
XI. H5 (236 μg/ml in PBS) lot 1 210406CS
XII. Amyloid-like misfolded protein mixture of H5 and OVA, heat-denatured (140 μg/ml in PBS) lot 2 fraction X 250506CS
XIII. Amyloid-like misfolded H5−OVA conjugate, heat-denatured (143 μg/ml in PBS) lot 2 fraction X 250506CS
XIV. H7 stock I (10.5 μg/ml in PBS) lot 2 05-2006CS (see above)
XV. Amyloid-like misfolded protein mixture of H7 and OVA, heat-denatured (10.5 μg/ml in PBS) lot 2 05-2006CS
XVI. Amyloid-like misfolded H7−OVA conjugate, heat-denatured (10.6 μg/ml in PBS) lot 2 05-2006CS
XVII. H7, stock II, 603 μg/ml (A/Netherlands/219/03, Protein Sciences Corp., Meriden, Conn., USA; catalogue number 3006, lot 112305, buffer: 10 mM Na-HPO₄, pH 7.0, 150 mM NaCl)

### Experimental Set-Up: Vaccine Preparation and Vaccination

For preparation of doses of cocktail vaccines, two solutions were prepared; 1. 20 μg/ml of each of the non-adjuvated antigens, 2. 2 μg/ml of misfolded H7 and 19 μg/ml of each of the other crossbeta-adjuvated antigens. The 2 μg/ml antigen cocktail stocks were prepared by 10-fold dilution of these stocks (solution 3. and 4.). The vaccines with 10 μg/ml non-adjuvated antigen/10 μg/ml crossbeta-adjuvated antigen and with 1 μg/ml non-adjuvated antigen/1 μg/ml crossbeta-adjuvated antigen were prepared by 1:1 mixing solutions 1. and 2., or 3. and 4., respectively (solution 5. and 6).

### Solution 1.

Antigens I, III, V, VIII, XI and XVII in PBS

### Solution 2.

Antigens II, IV, VI, VII, IX, X, XII, XIII, XV and XVI in PBS

For immunizations, female 7-9 weeks-old BalB/CAnNHSd mice (BalB/C, Harlan; six groups of five mice) (Animal Facility ‘Gemeenschappelijk Dierenlaboratorium’ GDL, Utrecht University, The Netherlands) were used. After approximately one week of adjustment to the environment, blood was drawn for collecting pre-immune serum at day −4. At day 0 each mouse received a subcutaneously injected vaccination of 500 μl according to the following scheme:
group a 100% non-adjuvated antigen cocktail, 10 μg/antigen/mouse (control)
group b 50% non-adjuvated/50% crossbeta-adjuvated antigen cocktail, 10 μg/antigen/mouse
group c 100% crossbeta-adjuvated antigen cocktail, 10 μg/antigen/mouse
group d 100% non-adjuvated antigen cocktail, 1 μg/antigen/mouse (control)
group e 50% non-adjuvated/50% crossbeta-adjuvated antigen cocktail, 1 μg/antigen/mouse
group f 100% crossbeta-adjuvated antigen cocktail, 1 μg/antigen/mouse

### Anti-Antigen Antibody Titer Determination with ELISA

With sera obtained at day 21 post-vaccination, antibody titers developed against each of the components of the vaccine cocktail were assessed using conventional ELISA techniques. The protocol was as described for CL3-KLH conjugate. For each group of mice a-f, sera were pooled and dilution series were prepared in PBS/0.1% Tween20. Coated antigens are given below. Dilution series of control antibodies recognizing CL3 peptide, E2, H5 or H7 are used as positive control in the ELISA's. H5 and H7 were coated at 2.5 μg/ml, E2 stock solution of Cedi-Diagnostics was diluted 100-fold before coating, CL3 peptide was coated at 10 μg/ml.

### Protein Solutions Used as Antigen in Antibody Titer ELISA's

Lyophilized E2 antigen, reconstituted according to the manufacturer's recommendation (Ceditest CSFV, Cedi-Diagnostics B. V., Lelystad, The Netherlands)
H5, 83 μg/ml H5 (A/Vietnam/1203/2004(H₅N1), Protein Sciences Corp., Meriden, Conn., USA), catalogue number 3006, lot 45-05034RA-2, buffer: 10 mM Na-HPO₄, pH 7.0, 150 mM NaCl
H7, 603 μg/ml H7 (A/Netherlands/219/03, Protein Sciences Corp., Meriden, Conn., USA), catalogue number 3006, lot 112305, buffer: 10 mM Na-HPO₄, pH 7.0, 150 mM NaCl
CL3 peptide (aliquot at 4° C., Lot BB1, Ansynth Service B. V., Roosendaal, Netherlands) NH₂—CSNDVSWHEWKRMYNKEYNG-COOH(CL3 peptide with N-terminal Cys extension)
OVA, 10 mg/ml in PBS (stock 060613NH-4° C.; catalogue number A-5503, Lot 14H7035, Sigma)
DOVA, 10 mg/ml in PBS (stock 060613NH-4° C.), heat-denatured

### Materials Used for Titer ELISA's

Microlon high-binding ELISA plates (Greiner, catalogue number 655092)
Coat buffer: 50 mM NaHCO₃, pH 9.6
Blocking reagent (Roche, catalogue number 11112589001)
Wash buffer: 50 mM Tris, 150 mM NaCl, 0.1% Tween20, pH 7.3
Mouse sera of individual animals, collected at day −4 and day 28 after vaccination
Anti-H₅N₁ mouse serum (‘ID-Lelystad (Dr L. Cornelissen) H5N1, gr5, 40106, 2.02.24150.00, nd2579jet, 180 μl ’)→control anti-H5 serum
Anti-E2 antibody, HRP conjugated (Art. 7610384, Lot 06C052, Cedi Diagnosticcs B. V., Lelystad, The Netherlands)
Anti-CL3 serum, mouse (ID-Lelystad (A. Antonis) YM30 III, c11c7e8)→control anti-CL3 serum
Mouse ascites fluid anti-chicken egg albumin (OVA), clone OVA-14, IgG1, (Sigma, A6075, lot 074K4768→control anti-OVA ascites
Mouse anti-H7N7 serum (‘muis-anti-H7N7, dpi:14 groep 6, 040106’, ID-Lelystad, Dr L. Cornelissen)→control anti-H7 serum
Binding buffer: 140 mM sodium chloride, 2.7 mM potassium chloride, 10 mM disodium hydrogen phosphate, 1.8 mM potassium di-hydrogen phosphate, pH 7.3 (PBS) with 0.1% Tween20
peroxidase-conjugated rabbit anti-mouse immunoglobulins (RAMPO, P0260, DAKOCytomation, Glostrup, Denmark)
PBS
H₂O₂: 35% v/v (Merck, Darmstadt, Germany)
OPD: 1,2-phenylenediamine (Merck, catalogue number 1.07243.0050, lot L937543-84)
Citrate/phosphate buffer pH 5.0
10% v/v H₂SO₄ in H₂O
Spectramax spectrophotometer for A_{490 nm} readings

### Results

### Anti-Antigen Titers in Mice Sera at Day 28 Post Vaccination

Balb/c mice were immunized with a cocktail vaccine with 1 or 10 μg antigens/animal. The cocktail contained E2, CL3, H5, H7 and OVA, and/or amyloid-like misfolded counterparts. Differences in crossbeta structure content between the vaccines for the six groups of mice is depicted in FIG. 11. The property to enhance tPA/plasminogen activity was assessed with antigen cocktails comprising 20 μg/ml of each of the antigens, with 0, 50 and 100% amyloid-like misfolded protein conformation, respectively. These relative contents of misfolded protein is reflected in the ability to activate tPA, which follows the same order. That also the 100% untreated antigen cocktail solution activates tPA to some extent is explained by the fact that at least untreated CL3 peptide, H5 and OVA display some characteristics of the presence of a small content of crossbeta structure even without treatment to induce such characteristics. The same order in signals as seen in the tPA activation assay, is observed with 10-fold dilutions of the cocktails in Congo red and ThT fluorescence assays (FIG. 11B, C).

With pooled mouse sera that were collected at day 28 post vaccination, titers against each of the individual untreated antigens present in the cocktail vaccine, were determined in the described ELISA set-up. In addition, titers against DOVA were also tested to be able to analyze the efficacy of the amyloid-like misfolded crossbeta structure adjuvated OVA.

No titers were found in the sera against coated free CL3 peptide or against untreated H5. Titers will again be analyzed at least 14 days after the mice received a second dose of the antigens, which may in this case be required to obtain a detectable titer.

Similar titers were developed against untreated H7 and the 1:1 mixture of untreated H7 and crossbeta-adjuvated H7, at both the 1 and 10 μg/animal doses (shown for the 10 μg/animal dose in FIG. 12A). The presence of a reasonable amount of H7 molecules with amyloid-like crossbeta structure conformation in the untreated H7 stock solution (See FIG. 9F, G) explains the observed immunogenicity.

When titers developed against coated E2 antigen are determined after a single-dose vaccination, mice that received 5 or 10 μg crossbeta-adjuvated E2, expressed in HEK293E cells, developed a titer (FIG. 12B). Apparently, after a single dose, no titer is elicited when mice are vaccinated with 1 μg E2/animal only. Interestingly, when mice are immunized with 100% amyloid-like misfolded E2 expressed in HEK293 cells, comprising a carboxy FLAG-tag-His-tag extension (group c), still an antibody titer against the untreated E2 expressed in Sf1 cells is developed. These observations demonstrate the beneficial use of the ‘adjuvation through crossbeta structure’ technology. Importantly, without the use of an adjuvant, no titer was elicited when untreated E2 was used as the antigen. With 50 or 100% crossbeta-adjuvated E2, titers are however developed, without the use of an adjuvant. In the six antigen cocktails OVA is included because of its immunogenic characteristics. Group a and d contain 20 and 2 μg OVA/ml, group c and f contain 20 and 2 μg/ml DOVA and an additional amount of DOVA due to the use of misfolded antigen-OVA conjugates. From the experiments shown in FIGS. 9A and B it is already learned that OVA comprises a relatively small, though not negligible amount of crossbeta structure, when compared to DOVA. From the immunization trials, it is now learned that this small amount can not elicit an anti-OVA or anti-DOVA titer (FIG. 12C, D). In contrast, DOVA in vaccine cocktails b, c and e elicits both anti-DOVA and anti-OVA titers. It is clear that a more potent titer is obtained against OVA, when OVA is part of the antigen cocktail (group b compared to group c; group e compared to group f (no titer). When 10 or 20 μg/ml DOVA is used, higher titers are reached than with 1 or 2 μg/ml DOVA. These observations show that DOVA comprising increased crossbeta structure content, is a more potent stimulator of immunogenicity than OVA, which only contains a minor crossbeta structure content. In addition, it is clear that OVA alone does not elicit a titer against OVA or DOVA, whereas when DOVA and OVA are combined, highest titers are obtained against both appearances of the antigen. When comparing the titers developed with antigen cocktail a and c, it is clear that the formation of amyloid-like misfolded protein conformation comprising crossbeta structure in OVA is sufficient to develop anti-antigen titers, without the use of an adjuvant. This provides direct evidence for the adjuvating property of crossbeta structure and the ‘adjuvation through crossbeta structure’ technology. It substantiates the immunogenic potential of the crossbeta structure conformation, which can be adopted by virtually every polypeptide, irrespective of the amino-acid sequence or the sequence length. More specifically, this OVA/DOVA example demonstrates that the combination of untreated antigen with crossbeta-antigen provides a potent stimulator of the immune system.

### Example 12

Crossbeta structure-adjuvation of an H5-subunit vaccine and H7-subunit vaccine induces higher antibody titers and crossbeta-H5 vaccination protects mice from challenge with lethal dose Avian Influenza Virus H₅N₁ (AIV) Vaccination study of mice with an H5-subunit vaccine

### Aim

Determination whether a subunit vaccine comprising H5-antigen of AIV with amyloid-like misfolded protein conformation (crossbeta structure-adjuvated H5) elicits antibody titers and protects mice from a challenge with AIV H₅N₁.

### Introduction

Influenza, in particular influenza caused by subtype influenza A (H5N1) poses an important pandemic threat. For this reason, maintaining the public health requires to prevent or treat the spread and infection with AIV, in particular H5N1. The key to meeting these goals is the development of safe and effective vaccines.

There are two genera of influenza virus: one including the influenza A and B viruses and the other the influenza C viruses. Influenza B and C are human viruses, whereas influenza A replicates and circulates in a wide range of avian and mammalian hosts. Of these, the influenza A viruses generally cause the most serious problems economically and in terms of human health. Influenza A viruses have segmented genomes of single-stranded negative sense RNA, which are encapsulated by a virally encoded nucleoprotein. The virus encodes two important viral surface antigens, haemagglutinin glycoprotein (HA or H) and neuraminidase (NA or N). The HA and NA viral surface antigens are classified serologically into subtypes; to date, 15 HA and 9 NA subtypes have been identified in nature. All subtypes circulate ubiquitously in wild waterfowl such as ducks, and these avian hosts provide the natural reservoir for all influenza A viruses. In these species, infections are generally localized to the intestinal tract, and high concentrations of virus are shed in the feces without causing disease. The HA is responsible for binding of virus particles to sialic acid-containing cell surface receptors and, after endocytosis, for mediating fusion of the viral and cellular membranes. It is a type I membrane glycoprotein containing a signal sequence that is removed post-translationally, a membrane anchor domain near the carboxy-terminus, and a short cytoplasmic tail. The HA is synthesized as a precursor of approximately 75 kDa that associates non-covalently as homo-trimers. The precursor polypeptides are post-translationally cleaved at a conserved arginine residue into two subunits, which are linked by a single disulfide bond. HA is the main vaccine antigen.

Thus far, all currently licensed influenza vaccines are generated in embryonated hen's eggs. Several well-recognized disadvantages to the use of such eggs as the substrate for influenza-vaccine production include the potential vulnerability of the supply of eggs, the long lead time required to scale up egg production, and the need to adapt new variants for high-yield growth in eggs, a process that can be time consuming and is not always successful. In addition, growth in eggs can result in selection of receptor variants that may not be optimal for protection against circulating strains. Moreover, recent studies in humans have indicated that an approach using an inactivated subvirion influenza A (H5N1) vaccine can results in serum antibody responses, including the formation of neutralizing antibodies, but that the response was incomplete and requires substantial amounts of antigen ^{6,7}. The frequency of antibody response was highest among subjects receiving doses of 45 μg or 90 μg. Among those who received two doses of 90 μg, neutralization antibody titers reached 1:40 or greater in 54 percent, and haemagglutination-inhibition titers reached 1:40 or greater in 58 percent. Neutralization titers of 1:40 or greater were seen in 43 percent, 22 percent, and 9 percent of the subjects receiving two doses of 45, 15, and 7.5 μg, respectively. No responses were seen in placebo recipients. Hence, influenza vaccines need to be improved.

An alternative method for production of influenza vaccine is expression of the main vaccine antigen, HA, by recombinant-DNA techniques. In a recent study, a subunit vaccine containing an HA (H1 and H3), derived from subtypes A/Panama/2007/99 (H3N2), A/New Caledonia/20/99 (H1N1), and B/Hong Kong/330/2001, and produced in insect cells by a recombinant baculovirus, was evaluated ⁸. This alternative avoids dependence on eggs, and the efficient protein expression, in this case using a baculovirus expression system. Baculovirus-expressed HA vaccine was safe and, compared with trivalent inactivated influenza vaccine, induced a better serum antibody responses to the H3 component when administered at doses of 45 μg or 135 μg of each HA. However, still, even when 135 μg was administered the number of responders was not complete (between 16 and 88%, depending on the subtype and amount of vaccine administered). These studies have used vaccines without adjuvant, since no good adjuvant is available for use with an influenza vaccine.

The purpose of the present study was to evaluate whether adjuvation of an HA subunit vaccine with crossbeta structure results in better immunogenicity and whether such vaccine protects mice upon challenge with AIV H5N1.

### Materials & Methods

### Immunization

Eigthy-eight female Balb/C mice were used. Mice were housed at the facilities of ID-Lelystad, The Netherlands. The mice were approximately six weeks at the start of the study. Mice were randomly allotted to a vaccine group or control group, each of the 11 groups comprised eight animals. The animals were allowed to eat (2185 RMH/B) and drink water ad libitum.

For immunizations of mice with untreated H5 and crossbeta-H5 stock solution used for vaccine formulation was the 236 μg/ml recombinant H5 stock in PBS (lot 1 210406CS, strain A/Vietnam/1203/2004) for the first vaccination and the 140 μg/ml H5 in 25 mM Tris pH 8.2, 500 mM NaCl (lot 2 fraction X 240506CS) solution for the second vaccination. For vaccine formulations three amyloid-like misfolded H5 preparations were used in a 1:1:1 ratio (see below). For five groups of mice (eight animals in each group), the following preparations were formulated, for doses of 5 μg H5/animal:
Group 2, 100% untreated H5
Group 3, 67% untreated H5/33% misfolded H5
Group 4, 33% untreated H5/67% misfolded H5
Group 5, 100% misfolded H5

For immunizations of mice with avian flu subunit H7 vaccine, vaccines were formulated using H7 lot 2 (160506CS, 10.5 μg/ml in PBS). For vaccine formulations comprising misfolded amyloid-like H7, the crossbeta-H7 obtained with Misfolding Methods I and II was used. Free H7 was misfolded, as well as H7 mixed with OVA and H7 conjugated with OVA using EDC/NHS coupling.

At day 0 and 21 the mice were immunized subcutaneously with 0.5 ml test sample, in the neck (day 0) and on the left side (day 21). Group T01 (control, #1.1-1.8) received test sample 1 (water, placebo), group T02 (#2.1-2.8) received sample 2 (5 μg untreated H5), group T03 (#3.1-3.8) received sample 3 (untreated H5 combined with 33% crossbeta-H5 modified by method I, II, III), group T04 (#4.1-4.8) received sample 4 (untreated H5 combined with 66% crossbeta-H5 modified by method I-III), group T05 (#5.1-5.8) received sample 5 (100% crossbeta-H5 modified by method I-III), group T06 (#6.1-6.8) received sample 6 (0.5 μg untreated H7), group T07 (#7.1-7.8) received sample 7 (untreated H7 combined with 33% crossbeta-H7 modified by method I, II), group T08 (#8.1-8.8) received sample 8 (untreated H7 combined with 33% crossbeta-H7 modified by method I, II), group T09 (#9.1-9.8) received sample 9 (100% crossbeta-H7 modified by method I, II), group T10 (#10.1-10.8) received sample 10 (vaccine, Gallimune® FLU H5N9, Lot F38785C, comprising inactivated avian influenza virus (H5N9, strain A/Turkey/Wisconsin/68) and group T11 (#11.1-11.8) received sample 11 (untreated H7 adjuvated with Specol). In the case of H5, in all cases 5 μg H5 was administered in total (untreated combined with modified) in PBS. In the case of H7, in all cases 0.5 μg H7 was administered in total in PBS.

### Challenge

On day 24 mice (groups T01, T03 and T10) were challenged by intranasal inoculation with 50 μl comprising a dose of 20 LD₅₀ (2*10⁵ TCID50/ml) of AIV H5N1 A/156/97/HK.

### Evaluation and Examination

During the course of the whole study the animals were monitored once each day (clinical score 0-4, dead, whereby (0) is defined as healthy, (1) is defined as rough pelt, vital, (2) rough skin, rolled up, less reactive, passive during handling, (3) is defined as rough pelt, rolled up, accelerated breathing, less reactive, passive during handling, and (4) is defined as rough pelt, rolled up, accelerated breathing, less reactive, passive during handling, unable to turn when laid on back). The total score was obtained by multiplying the score with the number of animals with this score. The number of animals suffering from respiratory problems was calculated by counting the number of animals in each group with a score of 3.

Blood samples for serum collection were taken from the tail vena on day 0, 21, 33, 42 (challenge) and 56. Blood was allowed to coagulate and sera was subsequently obtained after centrifugation (5′ at 3500 rpm). Sera were stored at −20° C.

Anti-H5 antibody titers and anti-H7 antibody titers were assessed with mouse sera collected at day −1 and day 33, twelve days after the second vaccination with the same dose of 5 μg H5/animal or 0.5 μg H7/animal. The H5 antigen used for vaccinations was expressed in HEK293E cells and comprised a carboxy-terminal FLAG-tag-His-tag extension. For titer determinations, H5 antigen of the same H5N1 strain purchased from Protein Sciences Corp. was used. For titer determinations concerning anti-H7 antibodies, recombinant H7 of the same strain (A/Netherlands/219/03) was purchased from Protein Sciences Corp.

With sera collected at day −1 and 33 of mice, titer determinations with respect to OVA were performed. For this purpose, sera of the eight animals in a group were pooled. Pooled pools of sera collected at day −1 were used as the negative control.

### Results

Eighty-eight mice (11 groups with 8 animals in each group) were used for the study. Each mouse was vaccinated at day 0 and 21 with placebo (water, group T01) or a subunit vaccine containing recombinantly produced structural glycoprotein H5 or H7 (groups T02-T09, T11) or an inactivated H5N9 virus vaccine (group T10). Mice in three groups (T01, T03 and T10) were challenged at day 42 by intranasal inoculation with 50 μl comprising a dose of 20 LD₅₀ (2*10⁵ TCID50/ml) of AIV H₅N1 A/156/97/HK.

In FIGS. 13A and B, titer determinations with pooled mouse sera collected at day 33 and with coated native H5 or H7 antigen of a different source are shown. Serum collected from group T01, that received placebo vaccine (i.e. water), do not contain anti-H5 or anti-H7 antibodies. In the mice that were vaccinated with various H5 vaccines, groups that received either untreated H5 without adjuvant (T02) or 100% crossbeta-adjuvated H5 developed minor titers, when compared to titers obtained after vaccination with 33% or 67% crossbeta-adjuvated H5. The latter two vaccines were as potent as inactivated H5N9 vaccine with respect to the elicited titer (FIG. 13A). These results demonstrate that including crossbeta-adjuvated antigen in the H5 vaccine results in a humoral immune response comparable to conventionally adjuvated inactivated virus vaccine. 100% crossbeta-adjuvated H5 antigen is a less immunogenic moiety compared to vaccines comprising a fraction of crossbeta-adjuvated antigen together with untreated/non-adjuvated antigen. Based on the titers, groups T01, T03 and T10 were subjected to an H5N1 virus challenge experiment.

With individual sera of mice of groups T01 (placebo), T03 (33% crossbeta-adjuvated H5) and T10 (inactivated H5N9 virus vaccine), that were subjected to H5N1 virus challenge, titers were determined against native H5 purchased from Protein Sciences Corp. (H5 used for vaccination was produced in-house in HEK293E cells). In FIGS. 13E and F titers are depicted for T03 and T10. No titers higher than obtained with pooled pre-immune serum were observed (not shown). For T03, titers in individual mice developed approximately in the order mouse 5=6=7>8>1=2=4>3>pre-immune pool=no titer. For T10, the order is mouse 6>2=4>3=8>1=5>7>pre-immune serum pool=no titer. These differences in the strength of the immune response are reflected in the degree of protection against the H5N1 virus challenge.

For the H7 study, the order in strength of the elicited anti-H7 antibody titers was H7/Specol>33% crossbeta-H7>67% crossbeta-H7>untreated/unadjuvated-H7˜100% crossbeta-H7˜placebo. So, again, it is clear that H7 adjuvated with crossbeta structure is an effective vaccine when antibody titers are considered. Again, similar to H5, untreated/non-adjuvated antigen and 100% crossbeta-adjuvated antigen without the use of native antigen are less immunogenic when compared to vaccines comprising 33% or 67% crossbeta-adjuvated antigen.

In FIG. 13C, titers against OVA in pooled sera obtained at day 33 post-vaccination with placebo or crossbeta-adjuvated H5 or untreated/non-adjuvated H5 are considered. For OVA, the order in developed anti-OVA titers is 100% crossbeta-adjuvated H5>67% crossbeta-adjuvated H5>33% crossbeta-adjuvated H5≧untreated/non-adjuvated H5˜pre-immune serum. The vaccines formulated with 33, 67 and 100% crossbeta-adjuvated H5 comprise an increasing amount of amyloid-like misfolded OVA (DOVA). When OVA is considered, natively folded antigen does not have to be part of the vaccine formulation. Apparently, in DOVA a reasonable density of native-like epitopes is exposed to which cross-reactive titers are elicited. The results thus prove that antigen with crossbeta structure are suitable for use in vaccines to elicit a desired immune response against a native antigen.

These studies together show that good antibody titers are achieved with the use of crossbeta structure as adjuvant, without the necessity to include a conventional adjuvant in the vaccine formulation. (Although not necessary, it is of course also possible to use a combination of a cross beta structure adjuvant and a conventional adjuvant, optionally at a lower dose than conventional doses, at some occasions) In non-vaccinated mice the first clinical signs were observed 3 days post infection. The signs were observed until the end of the study. From day 7 all animals in the non-vaccinated group suffered from respiratory symptoms. Six out of 8 animals died or had to be euthanized before the end of the study. One animal died on day 1 post infection, without signs of disease. All vaccinated animals survived the challenge (FIG. 13D). Upon vaccination with Gallimune® FLU H5N9, group T10, not all signs of infection could be prevented, however, none of the animals suffered from respiratory symptoms. Similarly, vaccination with crossbeta-adjuvated H5 resulted in a decrease in respiratory symptoms. With both vaccine regimens, all mice recovered completely. Adverse effects were observed in all mice upon vaccination and challenge with the commercially available vaccine (Table 25).

Table 26 shows the clinical scores after challenge. Table 27 shows the score of respiratory symptoms. Table 28 shows mortality upon challenge with H5N1. Haemagglutinin antibody titers are shown in Table 29.

Combined, these studies show that addition of crossbeta-adjuvated H5 or H7 to a vaccine increases the antibody titers and that vaccination of mice with crossbeta-adjuvated H5 reduces clinical symptoms and protects mice from dead as a consequence of infection with H5N1. Thus, addition of crossbeta structure allows protective immunogenicity.

### Example 13

### Crossbeta Structure-Adjuvated E2-Subunit Vaccine Protects Swine from Death after Challenge with Lethal Dose of Classical Swine Fever

### Virus

Vaccination study of swine with an E2-subunit vaccine

### Aim

Determination if a subunit vaccine comprising E2-antigen with amyloid-like misfolded protein conformation elicits antibody titers and protects swine from a lethal dose of classical swine fever virus.

### Introduction

Classical Swine Fever (CSF, synonym hog cholera) is a contagious and often fatal disease of swine, characterized by fever, hemorrhages, ataxia and immuno-suppression. The causative agent is classical swine fever virus (CSFV), a member of the genus Pestivirus of the family Flaviviridae. In many European countries, the virus is not endemic, but outbreaks of CSF occur periodically, and may cause large economic losses. After infection with CSFV, antibodies are raised against the structural glycoproteins E2 and Erns, and the non-structural protein NS3. E2 is the most immunogenic CSFV envelope protein and induces a neutralizing antibody response in pigs. Vaccines based on inactivated CSFV induce a fast and protective immune response. However, a drawback of these vaccines is that sera from vaccinated animals can not been distinguished from infected animals. A subunit vaccine against CSFV has been developed based on this envelope glycoprotein E2⁹. This subunit vaccine is thus a potential marker vaccine, as discrimination between vaccinated and infected pigs can be based on the detection of antibodies against Erns and/or NS3. The subunit vaccine contains E2 produced in insect cells has been tested for safety and efficacy^{5,10}. This E2-based subunit vaccine produces a protective immune response, albeit less fast. Hence some improvement to obtain a faster immune response is desired.

### Materials & Methods

### Preparation of Vaccines

Six groups of six pigs were immunized with 32 μg recombinant E2/animal or with placebo (H₂O, Test group T01). For a first vaccination, untreated E2, cyclic thermal misfolded E2 (Misfolding Method I) and alkyl-E2 (Misfolding Method IV) were used (lot 1 200406RS, 285 μg/ml in PBS) in the following ratios:
Group 1 placebo (H₂O)
Group 2 100% untreated E2
Group 3 50% misfolded E2 (Method I)/50% untreated E2
Group 4 50% misfolded alkyl-E2 (Method IV)/50% untreated E2
Group 5 25% misfolded E2 (Method I)/25% misfolded alkyl-E2 (Method IV)/50% untreated E2
Group 6 water-oil emulsion adjuvated E2

For the second immunization at day 21 recombinant E2 expressed by Sf1 cells has again been used, now from lot 2 030506RS, 280 μg/ml in PBS. E2 has been misfolded using four Misfolding Methods (see above). For vaccine formulations, a solution with a 1:1:1:1 ratio of the four misfolded E2 preparations was used, with a final E2 concentration of 225 μg/ml in PBS. Pig Test Groups 1, 2 and 6 (T01, T02, T06) received the same vaccine as during the first vaccination. Now, for the second vaccination, vaccines for pig Test Groups 3, 4 and 5 (T03, T04, T05) comprised 25%, 50% and 75% of the crossbeta-adjuvated E2. The dose was again 32 μg E2/pig.

### Immunization

Thirty-six male pigs were used. Pigs were housed at the facilities of ID-Lelystad, The Netherlands. The pigs were approximately 6 weeks old at vaccination, and were free of antibodies against CSFV, and other pestiviruses. Pigs were randomly allotted to a vaccine group or control group, each of the 6 groups (n=6) was housed in an isolated unit under high containment conditions. The animals were fed, and could drink water ad libitum. At day 0 and 21 the pigs were immunized intramuscular with 2.0 ml test sample, once on the left and once on the right, approximately 2 cm behind the ear. For the first immunization, group T01 (control, animals #114-119) received test sample 1 (water), group T02 (#120-125) received sample 2 (32 μg untreated E2), group T03 (#126-131) received sample 3 (16 μg untreated E2 combined with 16 μg E2 adjuvated with crossbeta method I), group T04 (#132-137) received sample 4 (16 μg untreated E2 combined with 16 μg E2 adjuvated with crossbeta method IV), group T05 (#138-143) received sample 5 (16 μg untreated E2 combined with 8 μg E2 adjuvated with crossbeta method I and 8 μg E2 adjuvated with crossbeta method IV), group T06 (#144-149) received sample 6 (32 μg untreated E2 adjuvated with double oil in water [DOE] as described ¹⁰. For the second immunization group T01 (control) received test sample 1 (water), group T02 received sample 2 (32 μg untreated E2), group T03 received sample 3 (24 μg untreated E2 combined with 8 μg E2 adjuvated with crossbeta method I/II), group T04 received sample 4 (16 μg untreated E2 combined with 16 μg E2 adjuvated with crossbeta method I/II), group T05 received sample 5 (8 μg untreated E2 combined with 24 μg E2 adjuvated with crossbeta method I/II), group T06 received sample 6 (32 μg untreated E2 adjuvated with double oil in water [DOE]¹⁰. One animal in group 6 received 1.5 ml instead of 2 ml.

### Challenge with CSFV Strain Brescia 456610

On day 42 nineteen pigs (all animals from groups 1, 3 and 6 and 1 animal from group 5, #143) were inoculated intranasally with a dose of 200 LD₅₀ of the highly virulent CSFV strain Brescia 456610.

### Evaluation and Examination

During the course of the whole study the animals were monitored once each day (clinical score 0-7). Clinical signs were defined as (1) malaise, which included the symptoms retarded growth, thin (waste), decreased appetite, no appetite, vomiting, slow/tired/reduced responsiveness, pig is unable to stand without assistance, general illness, shivering, (2) impairment of the respiratory system, which included coughing, sneezing, accelerated breathing, snoring or sniffing breathing, eye discharge (or runny eyes), conjunctivitis or nasal discharge (runny nose) and (3) bleeding, which included the symptoms, red spots on the ears, blood from the rectum, or pale. Each symptom was counted as 1. Anal temperature was measured starting 2 days before challenge until the end of the experiment (day 56). Fever was defined as a temperature above 40° C.

Blood samples for serum collection were taken on day 0, 2, 5, 9, 12, 16, 19, 26, 33, 42 (challenge) and 56. Blood was allowed to coagulate and sera was subsequently obtained after centrifugation (5′ at 3500 rpm). Sera were stored at −20° C.

Sera were tested for the presence of neutralizing antibodies with a neutralization peroxidase-linked assay (NPIA) using PK15 cells and a non-cytopathogenic virus using two monoclonal antibodies (batch V3: 030502, batch V4: 110702) reacting with different epitopes on E2¹¹. Serial dilutions of serum (in duplicate) were mixed with an equal volume of Eagle BSS containing 30±300 TCID50 CSFV (strain Brescia). After incubation for 1 h at 37° C. in a CO₂ incubator, approximately 25000 PK-15 cells per well were added. After four days, an IPMA was carried out, as described previously ^{10,12}.

Antibody titers were expressed as the reciprocal of the highest dilution that inhibited infection (100%) of the monolayer in 50% of the cell cultures. Titers <10 are interpreted as negative.

Sera were also tested for the presence of antibodies using an ELISA (Ceditest® CSFV and Ceditest® CSFV2.0, Cedi-Diagnostics, Lelystad, The Netherlands), according to instructions of the manufacturer.

The number of leucocytes and thrombocytes in EDTA blood samples was determined in a Medonic7 CA570 coulter counter. Leukopenia is defined as <8×10⁹ cells/1 l blood, and thrombocytopenia as <200×10⁹ cells/1 l blood.

### Results

Thirty-six pigs (6 groups with 6 animals in each group) were used for the study. Each pig was vaccinated at day 0 and 21 with a control vaccine (water, group T01) or a subunit vaccine containing recombinantly produced structural glycoprotein E2 (groups T02-T06), and subsequently challenged with CSFV (strain Brescia 456610) as described in the Materials & Methods section.

Neutralizing antibody titers were determined (Table 29). Titers in animals vaccinated with E2 adjuvated with crossbeta structure (T03 and pig #143 of T05) were significantly higher on day 26, 33 and 42 as compared with the control group (T01). Pig #127 in T03 did hardly develop an NPLA titer.

Animals in Group T01 (placebo), group T03 (50% crossbeta-E2 Method I/25% crossbeta-E2 Method I and II), Group T05, pig #143 (50% crossbeta-E2 Method I/IV/75% crossbeta-E2 Method I and II) were involved in the challenge experiment. All animals that were immunized with placebo/water (control Group T01) died (FIG. 14). All animals, with one exception (T03, pig #127, hardly an NPLA titer), that received an E2-vaccine, whether adjuvated with crossbeta structure or DOE, survived (FIG. 14). The animals immunized with E2 adjuvated with DOE showed no clinical signs of infection. The animals receiving E2 adjuvated with crossbeta structure did show signs of infection, however, the signs were less severe when compared to animals in T01 (Table 30). Less breathing problems and less bleeding were seen in animals vaccinated with E2 adjuvated with crossbeta-structure, when compared to T01 (placebo). With pigs in T01, bleedings were seen in six out of six pigs. Of the seven crossbeta-E2 vaccinated pigs, four out of seven pigs had bleedings after CSFV challenge. The average duration of the bleedings was 1.8 days in the crossbeta-E2 vaccinated pigs versus 3.2 days in the placebo-treated pigs in T01. In T03 (crossbeta-E2 vaccine), amongst other pigs, pig #127, that did hardly develop an NPLA titer, suffered from bleedings.

The clinical scores and analysis of blood samples is illustrated below. Table 31 shows clinical scores in the post challenge phase. Table 32 shows the measurements of the temperature during the challenge.

In the control group (T01) and the group immunized with E2 adjuvated with crossbeta structure observed leucopenia (100%, 87%) and thrombocytopenia (both 100%) was significant (Table 34), as compared with the group that received E2 adjuvated with DOE (0% leucopenia, 17% thrombocytopenia). Pig #127 in the T03 group displayed very strong leucopenia and thrombocytopenia, most likely related to the absence of an NPLA titer.

These studies show that partial protection, i.e. survival with reduction in clinical symptoms, against a lethal dose of CSFV, tested in a severe challenge experiment, is obtained upon vaccination with E2 adjuvated with crossbeta structure.

### Example 14

### Immunization of Chickens with Ovalbumin Comprising Crossbeta Structure Induces Breaking of Tolerance and Results in Formation of Auto-Antibody Titers

### Materials and Methods

### Misfolding of OVA

Samples used for immunization of chicken were identical to those used for immunization of mice as described above (example 12).

### Immunization

Eighty-eight male LSL Lohman chicken of approximately 3 weeks old were used. At day 0 and 21 the chicken were immunized intramuscular with 0.5 ml test sample, in the left (day 0) and right thigh (day 21). Group T01 (control, #4601-4608) received test sample 1 (water, placebo), group T02 (#4609-4616) received sample 2 (5 μg untreated H5), group T03 (#4617-4624) received sample 3 (untreated H5 combined with 33% crossbeta-H5 modified by method I, II, III), group T04 (#4625-4632) received sample 4 (untreated H5 combined with 66% crossbeta-H5 modified by method I-III), group T05 (#4633-4640) received sample 5 (100% crossbeta-H5 modified by method I-III), and group T10 (#4673-4680) received sample 10 (vaccine, Gallimune® FLU H5N9, Lot F38785C, comprising inactivated avian influenza virus (H5N9, strain A/Turkey/Wisconsin/68). In all cases 5 μg H5 was administered in total (untreated combined with modified) in PBS.

Blood samples for serum collection were taken from the wing vena on day −1, 21, 33. Blood was allowed to coagulate and sera was subsequently obtained after centrifugation (5′ at 3500 rpm). Sera were stored at −20° C. Anti-OVA antibody titers were assessed with sera collected at day 0 and day 33 using a standard ELISA. With sera collected at day −1 and 33 of chicken, titer determinations with respect to DOVA were performed. For this purpose, sera of the eight animals in a group were pooled. Pooled pools of sera collected at day −1 were used as the negative control.

### Results

In FIG. 15, titer determinations with pooled chicken sera are shown. Serum collected from group T01, that received placebo vaccine (i.e. water), do contain detectable amounts of anti-OVA antibodies. The chicken that were vaccinated with various vaccines containing OVA developed higher anti-OVA titers. Most notably, immunization with vaccines containing 33% crossbeta-adjuvated sample resulted in increased anti-OVA titers. Similarly, the addition of adjuvant induced anti-OVA antibodies. These results demonstrate that including crossbeta-adjuvated OVA-antigen in the vaccine results in a humoral auto-immune response. Given this potency of crossbeta structure to break tolerance, vaccines against self-antigens are developed. Such vaccines are used against diseases or purposes other than infections, for example for the induction of antibodies to LHRH for immunocastration of boars, or for use in preventing graft versus host (GvH) and/or transplant rejections.

### Description of the Tables

The compounds listed in Table 1 and the proteins listed in Table 2 all bind to polypeptides with an amyloid-like non-native fold. In literature, this non-native fold has been designated as protein aggregates, amorphous aggregates, amorphous deposit, tangles, (senile) plaques, amyloid, amyloid-like protein, denatured protein, amyloid oligomers, amyloidogenic deposits, cross-β structure, β-pleated sheet, cross-β spine, plaque, denatured protein, cross-β sheet, β-structure rich aggregates, infective aggregating form of a protein, unfolded protein, amyloid-like fold/conformation and perhaps alternatively. The common theme amongst all polypeptides with a non-nativean amyloid-like fold, that are ligands for one or more of the compounds listed in Table 1 and 2, is the presence of a cross-β structure conformation.

The compounds listed in Table 1 and 2 are considered to be only a subset of all compounds known to day to bind to non-native protein conformations. The lists are thus non-limiting. More compounds are known today that bind to amyloid-like protein conformation. For example, in patent AU2003214375 it is described that aggregates of prion protein, amyloid, and tau bind selectively to polyionic binding agents such as dextran sulphate or pentosan (anionic), or to polyamine compounds such as poly (Diallyldimethylammonium Chloride) (cationic). Compounds with specificity for non-native folds of proteins listed in this patent and elsewhere are in principle equally suitable for methods and devices disclosed in this patent application. Moreover, also any compound or protein related to the ones listed in Table 1 and 2 are covered by the claims. For example, point mutants, fragments, recombinantly produced combinations of cross-β structure binding domains and deletion- and insertion mutants are part of the set of compounds as long as they are capable of binding to a cross-β structure (i.e. as long as they are functional equivalents) Even more, also any newly discovered small molecule or protein that exhibits affinity for the cross-β structure fold can in principle be used in any one of the methods and applications disclosed here.

The compounds listed in Table 3 are also considered to be part of the ‘Cross-β structure pathway’, and this consideration is based on literature data that indicates interactions of the listed molecules with compounds that likely comprise the cross-β structure fold but that have not been disclosed as such. The tables 4 to 34 depict results of the examples.

### LEGENDS TO THE FIGURES

FIG. 1. Activation of factor XII by adjuvant kaolin and by peptide aggregates with cross-β structure conformation.

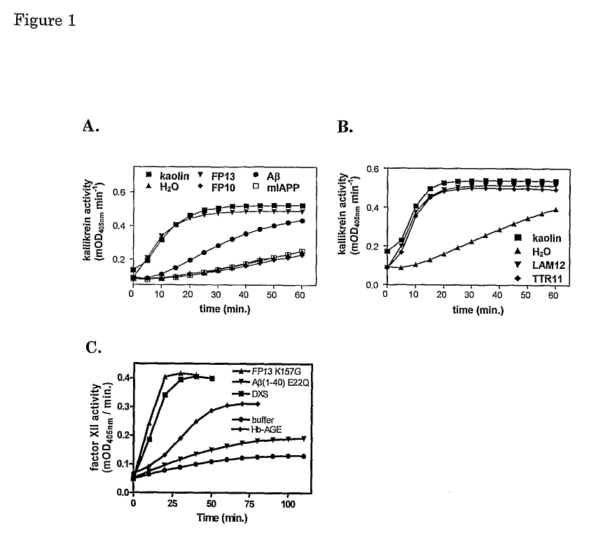

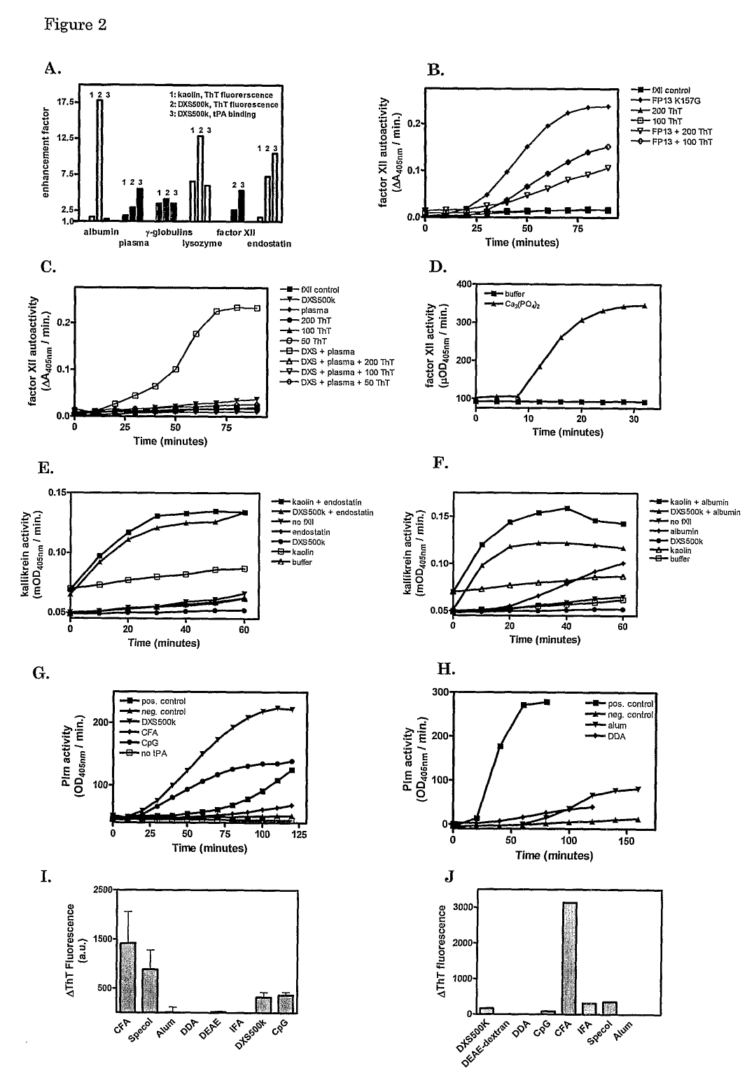

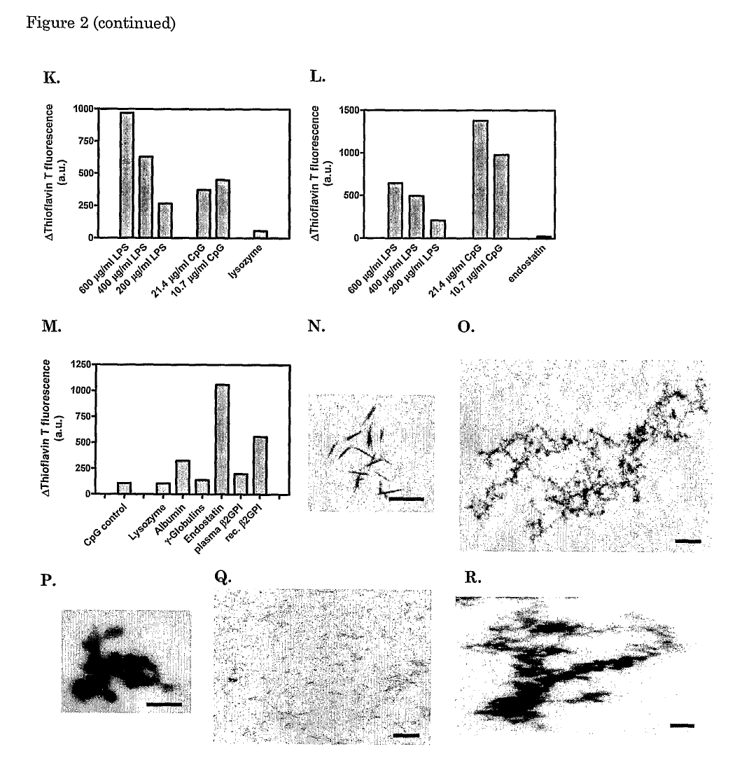

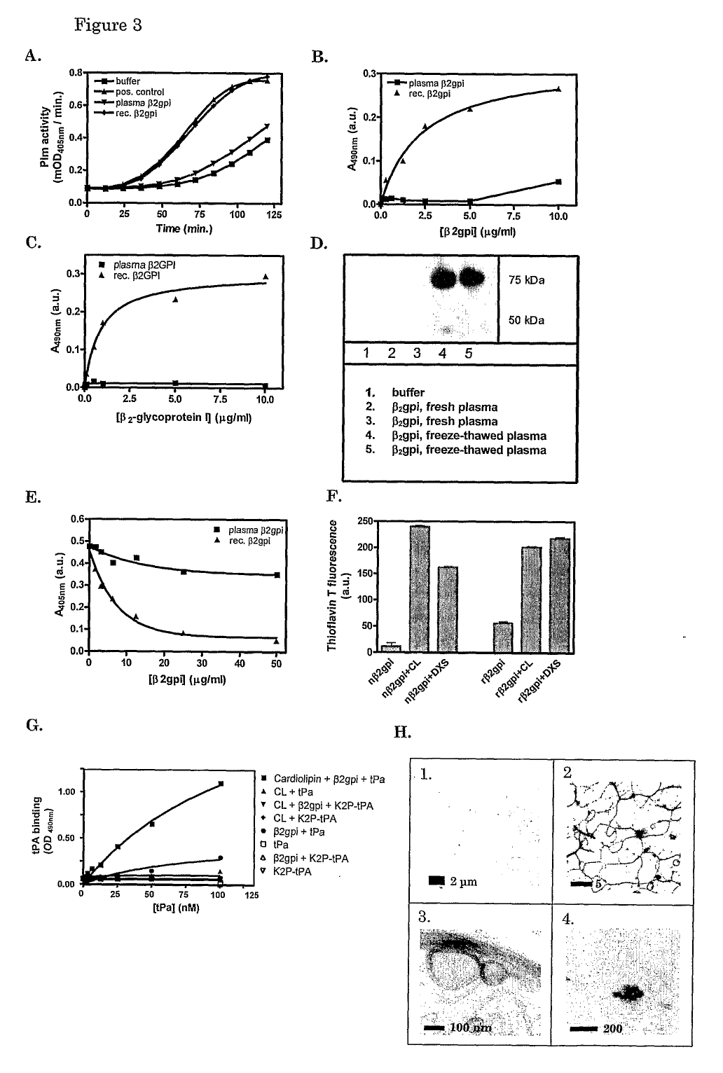

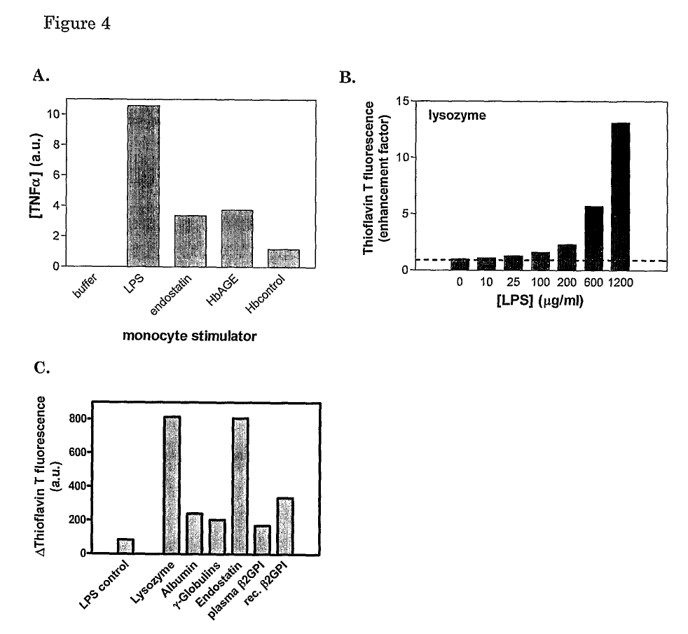

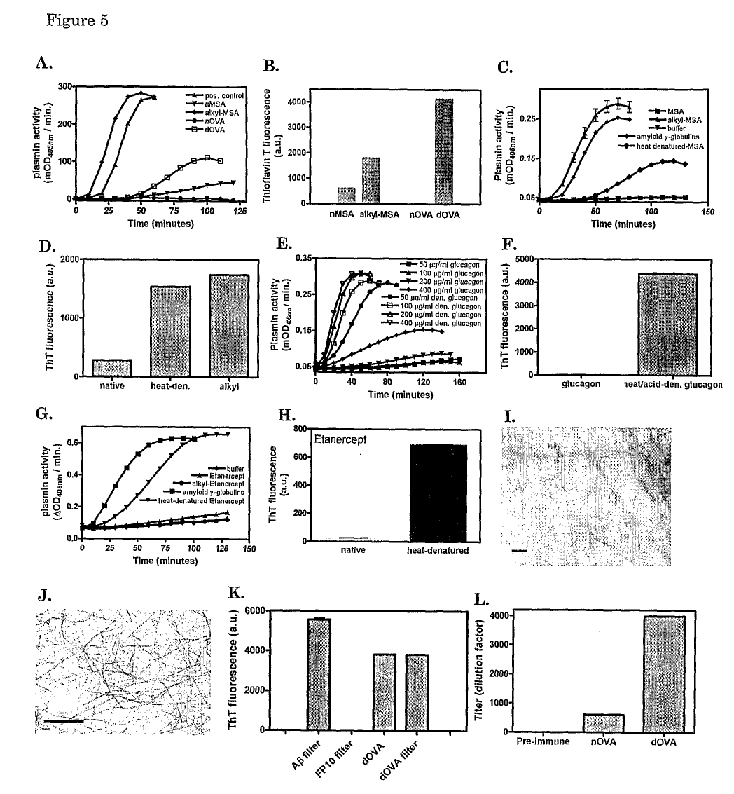

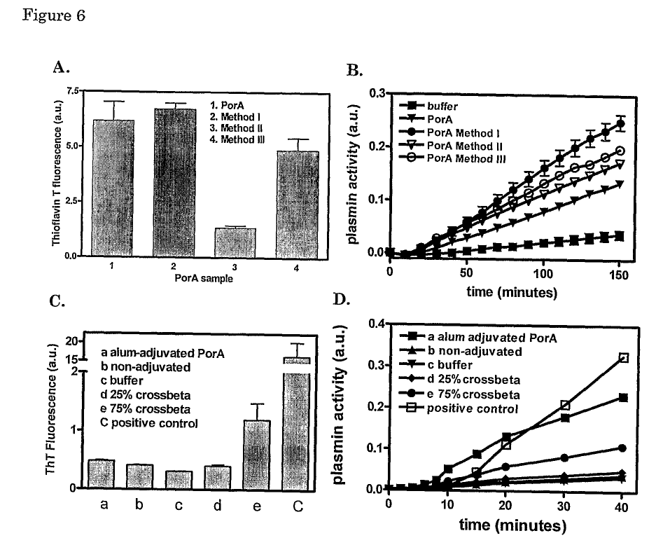

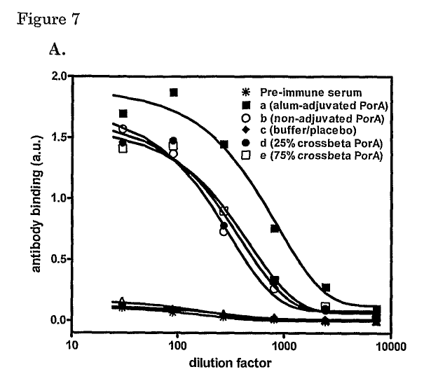

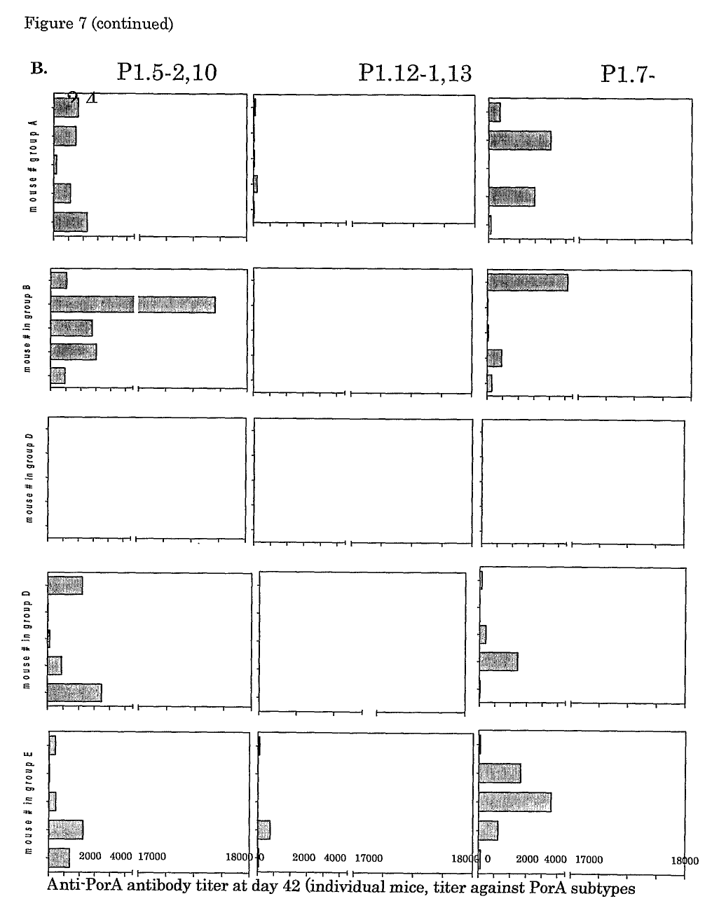

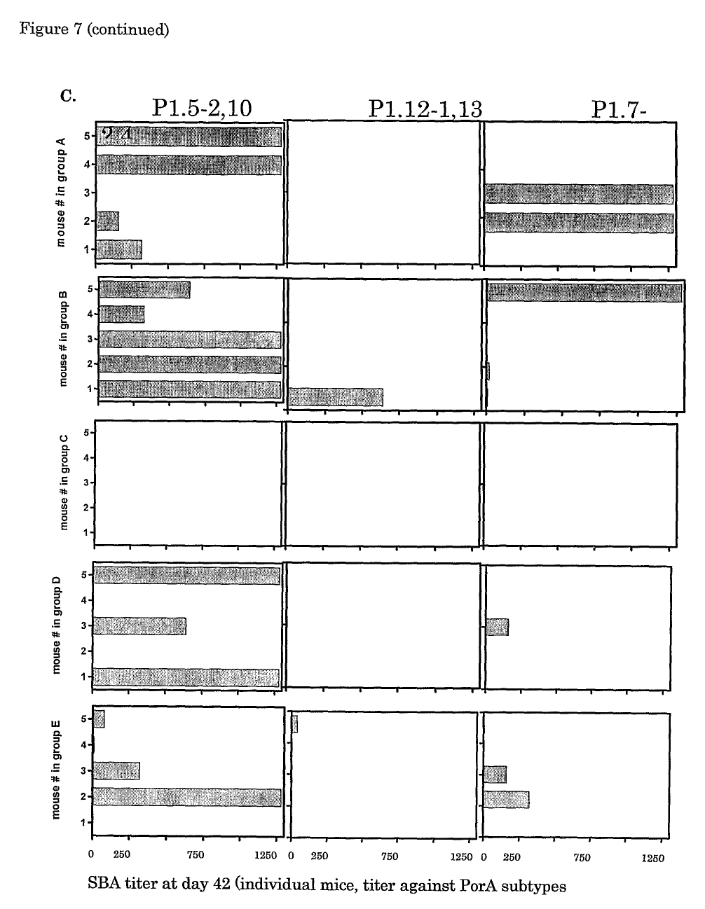

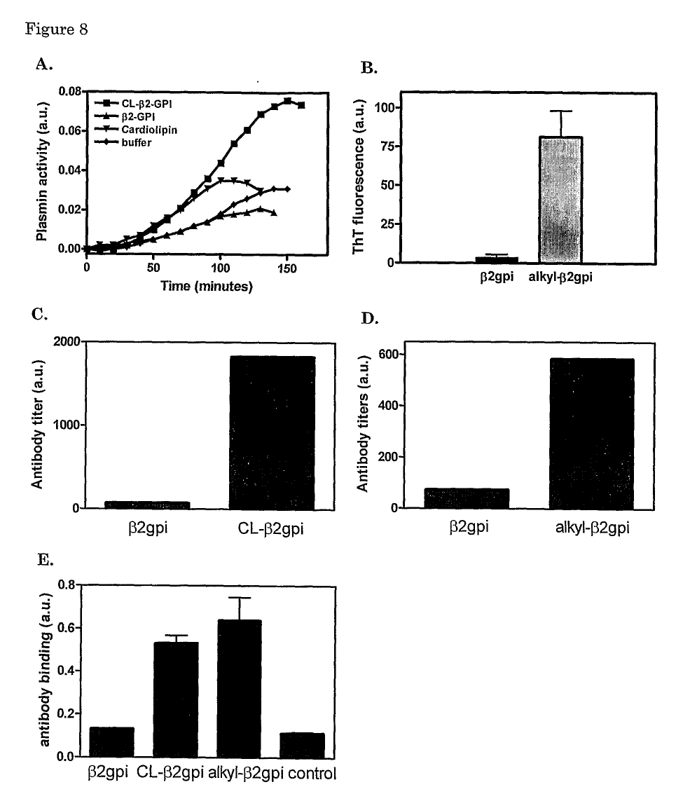

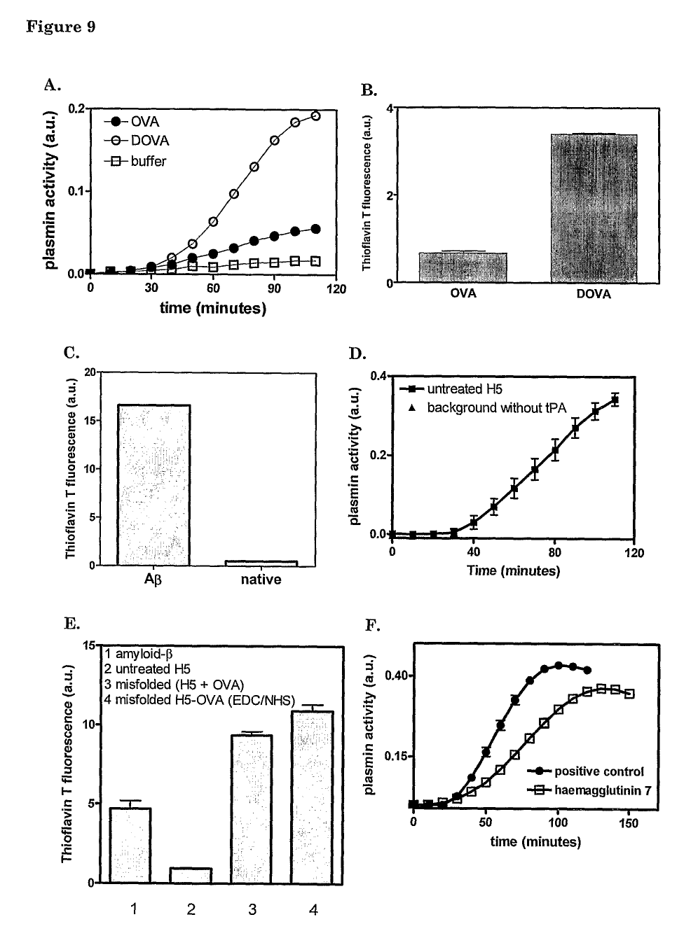

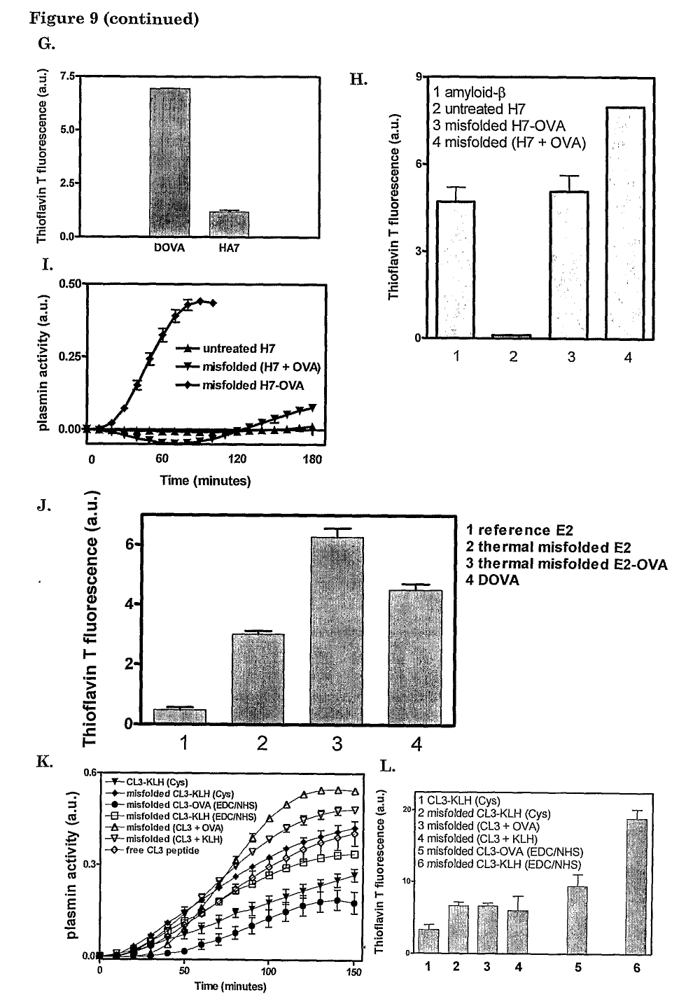

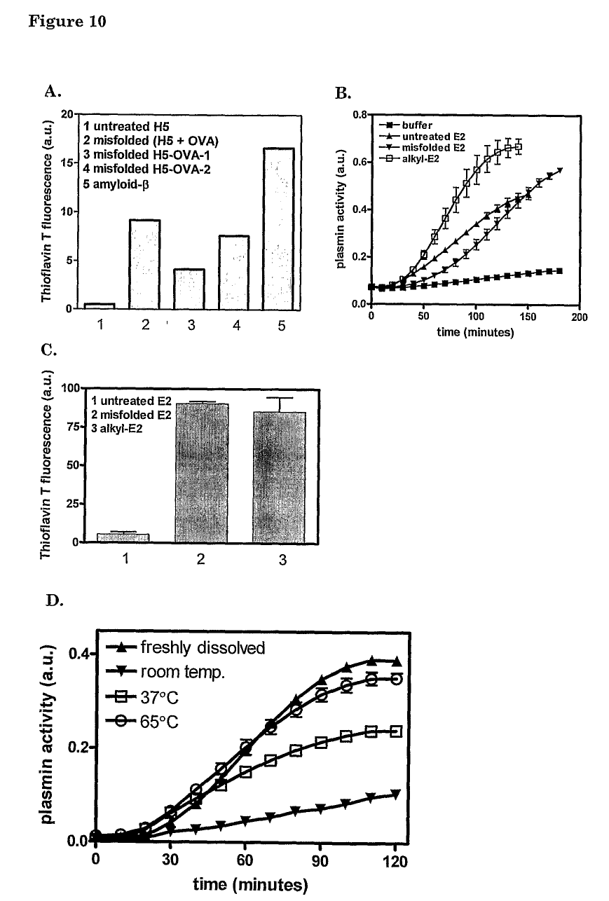

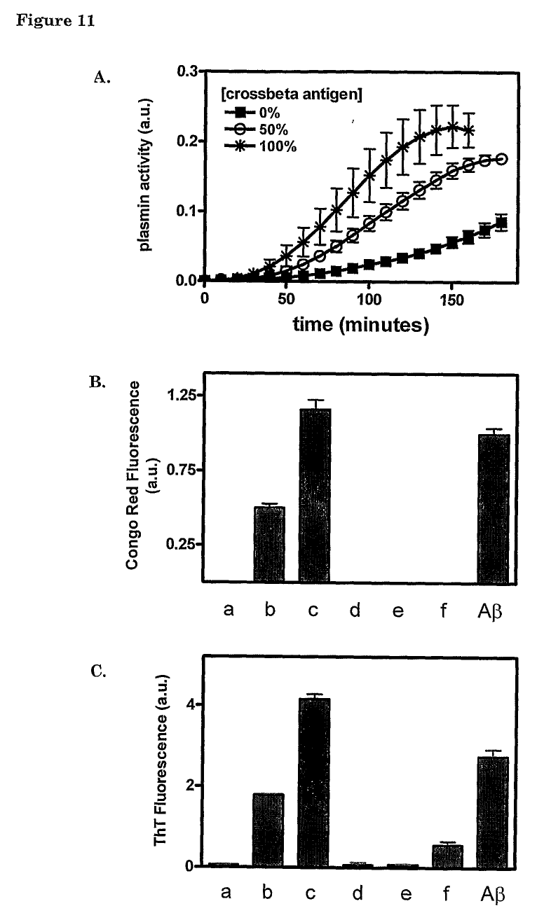

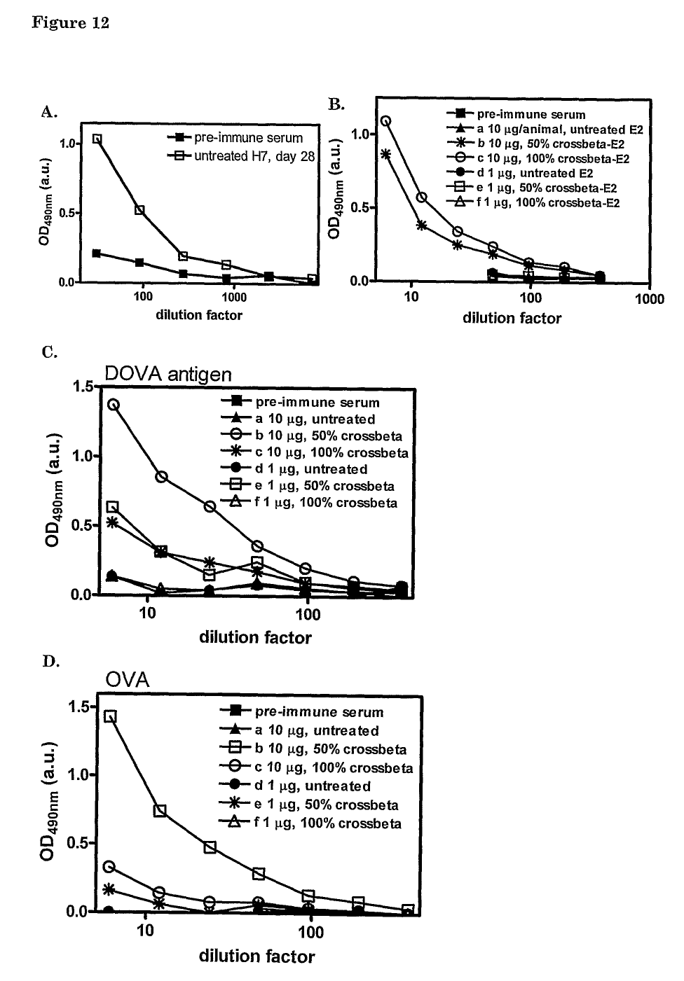

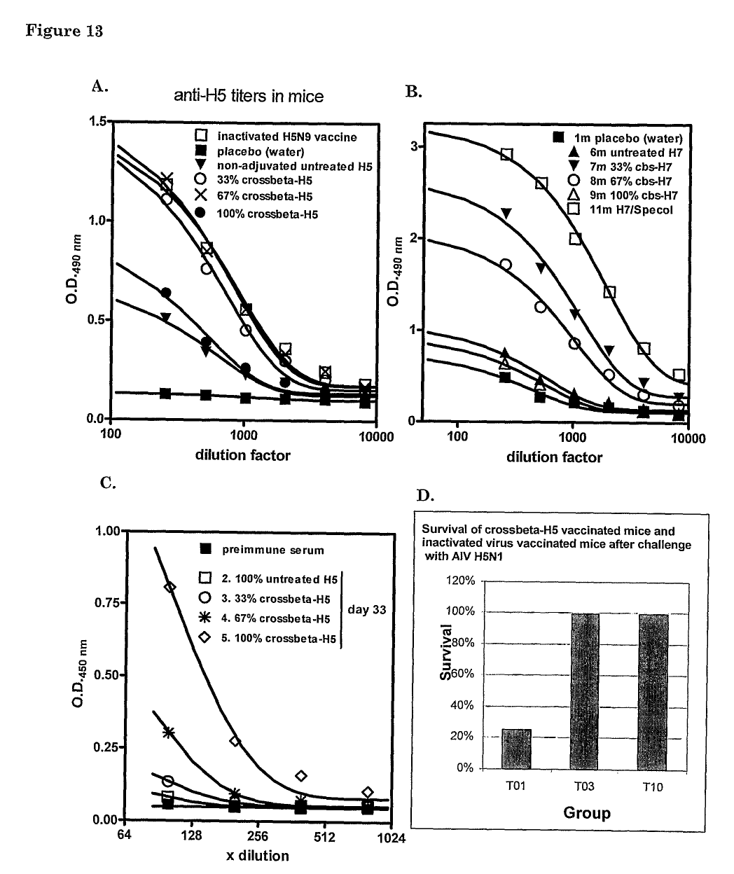

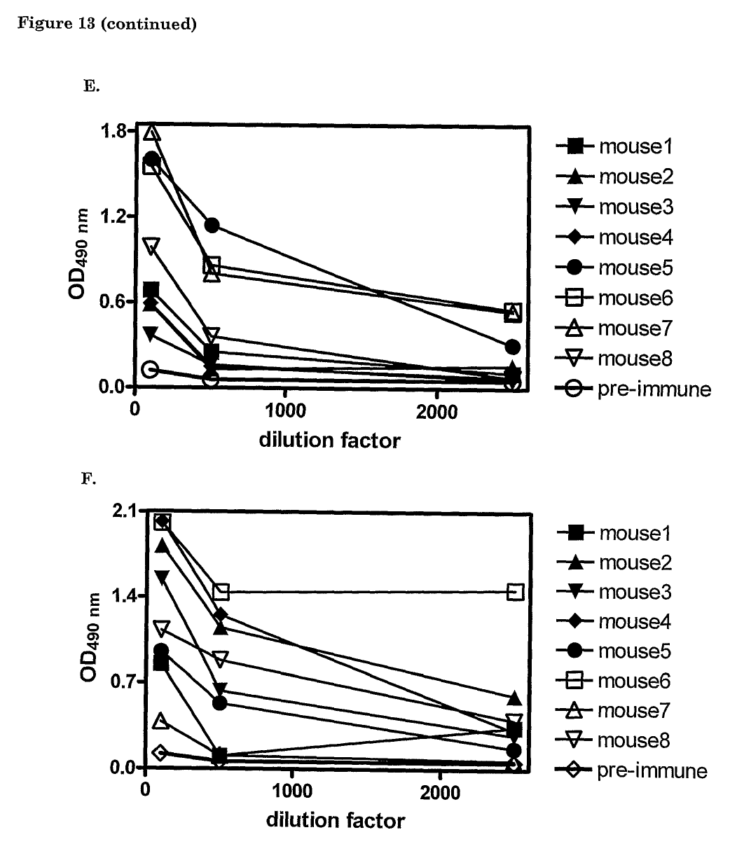

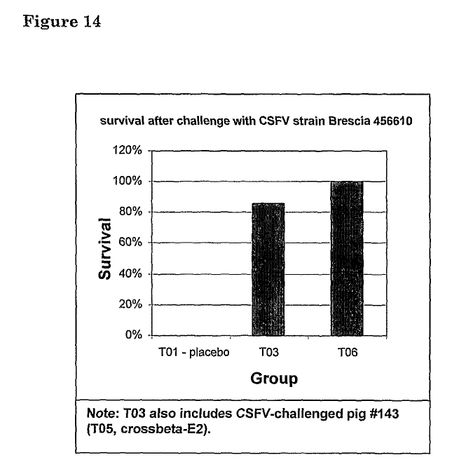

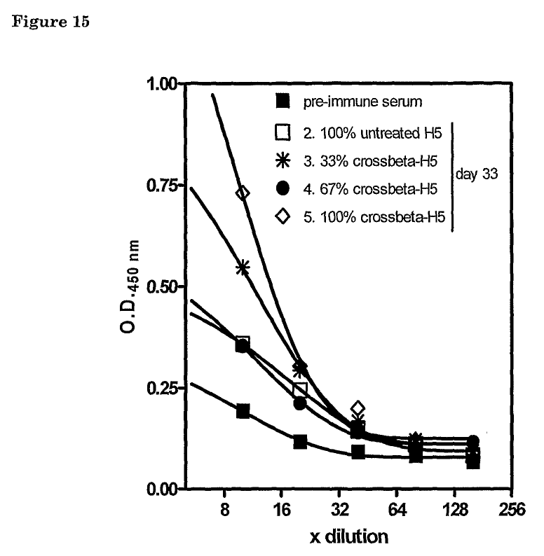

A. Like kaolin, amyloid-like peptide aggregates of FP13 and Aβ stimulate the activation of factor XII, as detected by the conversion of Chromozym-PK, upon formation of kallikrein from prekallikrein by activated factor XII. Buffer control and non-amyloid controls fibrin fragment FP10 and non-amyloid murine islet amyloid polypeptide mIAPP do not activate factor XII. B. Like FP13 and Aβ, also cross-β structure conformation rich peptides laminin (LAM12) and transthyretin (TTR11) stimulate factor XII activation, to a similar extent as kaolin. C. Autoactivation of factor XII is established by incubating purified factor XII with DXS500k or with various amyloid-like protein aggregates with cross-β structure conformation, in the presence of chromogenic substrate S-2222.

FIG. 2: Adjuvants induce amyloid-like properties in various proteins.

A. Adjuvants DXS500k and kaolin induce ThT fluorescence, and adjuvant DXS500k induces tPA binding properties in various proteins after overnight incubation, as measured in an ELISA with immobilized proteins with or without DXS500k. ThT fluorescence or tPA binding with proteins incubated with DXS500k or kaolin is given as a multiple of the fluorescence or tPA binding observed when DXS500k and kaolin were omitted during protein incubations (‘enhancement factor’). B. In a chromogenic factor XII autoactivation assay amyloid fibrin-derived peptide FP13 K157G stimulates factor XII autoactivation, which is inhibited by amyloid specific dye ThT. C. Adjuvant DXS500k is only a stimulatory factor for factor XII activation when 80× diluted plasma is present. Activation of factor XII by DXS500k and plasma proteins is inhibited by ThT. Factor XII activation was measured in a chromogenic assay. D. In the presence of 60% v/v plasma, adjuvant Ca₃(PO₄)₂ precipitate activates factor XII, as detected by measuring the conversion of chromogenic substrate S2222. E-F. Factor XII is only then effectively activated when both adjuvants kaolin or DXS500k and either 1 mg ml⁻¹ endostatin (E), or albumin (F) are included in the assay mix. Activation of factor XII in the presence of prekallikrein and high molecular weight kininogen was determined by measuring conversion of chromogenic kallikrein substrate Chromozym-PK. G-H. Adjuvants DXS500k, CpG, complete Freund's adjuvant (G), alum and DDA (H) induce activation of tPA and Plg, as determined by measuring the conversion of chromogenic plasmin substrate S2251. Positive control was 100 μg ml⁻¹ amyloid γ-globulins, negative control was buffer. I. Incubation of 80× diluted plasma with indicated adjuvants results in ThT fluorescence for complete Freund's adjuvant (CFA), Specol, DXS500k and CpG, whereas DEAE-dextran, incomplete Freund's adjuvant, alum and DDA have no effect. Samples were diluted 40× for the ThT measurements. J. In a similar experiment 20× diluted plasma was incubated with the indicated series of adjuvants. After 160× dilution, DXS500k, CpG, CFA, IFA and Specol induce ThT fluorescence. K-L. Exposure of 1 mg ml⁻¹ lysozyme (K) or endostatin (L) to indicated concentration series of LPS or CpG induces an enhanced ThT fluorescence signal. M. Exposure of 1 mg ml⁻¹ albumin, endostatin, plasma β2GPI or rec. β2GPI to 21.4 μg ml⁻¹ CpG results in increased ThT fluorescence with approximately a factor 2 to 10. With these assay conditions no effect is seen with lysozyme and γ-globulins. N-R. TEM images of CpG (N.), lysozyme (O.), lysozyme exposed to CpG (P.), DXS500k (Q.) and lysozyme exposed to DXS500k (R.). The scale bar represents 200 nm.

FIG. 3: Binding of factor XII and tPA to β₂-glycoprotein I and binding of anti-β₂GPI auto-antibodies to recombinant β₂GPI.

A. Chromogenic plasmin assay showing the stimulatory activity of recombinant β₂GPI on the tPA-mediated conversion of Plg to plasmin. The positive control was amyloid fibrin peptide FP13. B. In an ELISA, recombinant β₂GPI binds to immobilized tPA, whereas β₂GPI purified from plasma does not bind. The k_{D} is 2.3 μg ml⁻¹ (51 nM). C. In an ELISA, factor XII binds to purified recombinant human β₂GPI, and not to β₂GPI that is purified from human plasma, when purified factor XII is immobilized onto ELISA plate wells. Recombinant β₂GPI binds with a k_{D} of 0.9 μg ml⁻¹ (20 nM) to immobilized factor XII. D. Western blot incubated with anti-human factor XII antibody. The β₂GPI was purified either from fresh human plasma or from plasma that was frozen at −20° C. and subsequently thawed before purification on a β₂GPI affinity column. Eluted fractions are analyzed on Western blot after SDS-PA electrophoresis. When comparing lanes 2-3 with 4-5, it is shown that freezing-thawing of plasma results in co-purification of factor XII together with the β₂GPI. The molecular mass of factor XII is 80 kDa. E. In an ELISA recombinant β₂GPI efficiently inhibits binding of anti-β₂GPI auto-antibodies to immobilized β₂GPI, whereas plasma β₂GPI has a minor effect on antibody binding. Anti-β₂GPI auto-antibodies were purified from plasma of patients with the autoimmune disease Anti-phospholipid syndrome. F. Exposure of 25 μg ml⁻¹ β₂GPI, recombinantly produced (rβ2GPI) or purified from plasma (nβ₂GPI), to 100 μM cardiolipin vesicles or to 250 μg ml⁻¹ dextransulphate 500,000 Da (DXS) induces an increased fluorescence of ThT, suggestive for an increase in the amount of cross-β structure conformation in solution. Signals are corrected for background fluorescence of cardiolipin, DXS, ThT and buffer. G. Binding of tPA and K2P-tPA to β₂GPI immobilized on the wells of an ELISA plate, or to β₂GPI bound to immobilized cardiolipin is assessed. B₂GPI contacted to cardiolipin binds tPA to a higher extent than β₂GPI contacted to the ELISA plate directly. K2P-tPA does not bind to β₂GPI. TPA does not bind to immobilized cardiolipin. H. Transmission electron microscopy images of 400 μg ml⁻¹ purified plasma β2GPI alone (1) or contacted with 100 μM cardiolipin (2, 3) and of 400 μg ml⁻¹ purified recombinant β2GPI (4).

FIG. 4: Synthesis of TNFα RNA in monocytes after stimulation with cross-β structure conformation rich compounds and LPS, which acts as a denaturant.

A. Cultured U937 monocytes were incubated for 1 h with buffer, LPS, amyloid endostatin, amyloid Hb-AGE or control Hb. Upregulation of TNFα RNA was assessed by performing RT-PCR with RNA isolated form the monocytes and TNFα primers. Amounts of TNFα cDNA after RT-PCR were normalized for the amounts of ribosomal 18S cDNA, obtained with the same RNA samples. In monocytes incubated with buffer no TNFα RNA is detected. Endostatin and Hb-AGE induce approximately 30% of the TNFα RNA expression, when compared to LPS, whereas the TNFα RNA expression induced by control Hb is approximately threefold lower. B. Exposure of 1 mg ml⁻¹ lysozyme to 0-1200 μg ml⁻¹ LPS results in a 1.1 up to a 13.1 fold increase of ThT fluorescence with respect to lysozyme incubated with buffer only, indicative for the denaturing capacity of LPS, resulting in amyloid-like structures in lysozyme. Standard deviations were typically less than 10% (not shown). C. Exposure of 1 mg ml⁻¹ lysozyme, albumin, endostatin, γ-globulins, plasma β2GPI or rec. β2GPI to 600 μg ml⁻¹ LPS results in increased ThT fluorescence with approximately a factor 2 to 10.

FIG. 5. Amyloid-like cross-β structure conformation in alkylated murine serum albumin and in heat-denatured ovalbumin, murine serum albumin, human glucagon and Etanercept.

A. Plg-activation assay with plasmin activity read-out using chromogenic substrate S-2251. Activating properties of reduced and alkylated murine serum albumin (alkyl-MSA) and heat-denatured OVA (dOVA) are compared with amyloid γ-globulins (positive control), buffer (negative control), and native albumin and OVA (nMSA, nOVA). B. Thioflavin T fluorescence assay with native and denatured MSA and OVA. C. Plg-activation assay for comparison of reduced and alkylated MSA and heat-denatured MSA. D. Thioflavin T fluorescence assay with reduced/alkylated MSA and heat-denatured MSA. E. Plg-activation assay with concentration series of heat/acid denatured glucagon. F. Thioflavin T fluorescence assay with native and heat/acid denatured glucagon. G. Comparison of the tPA activating properties of heat-denatured Etanercept, native Etanercept and reduced/alkylated Etanercept. H. Thioflavin T fluorescence of native and heat-denatured Etanercept. I. TEM image of heat-denatured OVA. The scale bar represents 200 nm. J. TEM image of heat/acid-denatured glucagon. The scale bar represents 1 μM. K. Thioflavin T fluorescence assay showing that filtration through a 0.2 μm filter of denatured OVA does not influence the fluorescence enhancing properties. L. Titer determination of anti-nOVA antibodies in pooled sera of mice immunized with nOVA or dOVA. Titer is defined as the sera dilution that still gives a signal above the background value obtained with 10 times diluted pre-immune serum. The titer for the nOVA immunized mice was 610*, for the dOVA immunized mice 3999*. After one week no titer was detected in both groups. The 6.6 times increased titer seen in the dOVA immunized mice points to a higher immunogenic activity of denatured OVA with cross-β structure conformation.

FIG. 6. Detection of amyloid-like misfolded protein in PorA preparations.

A. Stock solutions of PorA and PorA subjected to either of three protein misfolding Methods I-III are applied 400-fold diluted in a ThT fluorescence assay. B. The same PorA samples are tested for their potential to activate tPA/plasminogen in a chromogenic plasmin assay. Samples were diluted 400-fold. C. ThT fluorescence assay with 10-fold diluted PorA vaccine preparations (1 μg/ml PorA in assay). D. tPA/plasminogen activation assay with 20-fold diluted PorA vaccine preparations (0.5 μg/ml PorA in assay).

FIG. 7. Anti-PorA antibody titer determinations and serum bactericidal antibody titer determinations.

A. At day 21 post vaccination, pooled sera of each group of mice were analyzed for their anti-trivalent PorA antibody titers, using the trivalent PorA antigen used for vaccination at day 0. B. Anti-PorA antibody titers, determined with individual sera collected at day 42 post vaccination at day 0 and day 14 post vaccination at day 21. Antigens used were the three PorA subtypes that built up the trivalent vaccine. C. Serum bactericidal antibody (SBA) titers, determined with sera as in B.

FIG. 8. Immunization of mice with human crossbeta-β2-glycoprotein I results in antibody titers against self- and non-self antigen.

A. Exposure of human β2gpi to cardiolipin renders the mixture with tPA/plasminogen activating properties, indicative for the presence of crossbeta structure, as determined in a chromogenic tPA/plasminogen activation assay with a plasmin substrate. B. Alkylation of cysteine residues in human β2gpi introduces crossbeta structure as determined in a ThT fluorescence enhancement assay. C. Mice immunized with a mixture of cardiolipin and human β2gpi develop an antibody titer against untreated human β2gpi antigen. D. Similar to cardiolipin-β2gpi with crossbeta structure, also human alkyl-β2gpi with crossbeta structure elicits an antibody titer against the untreated human antigen. E. Mice immunized with human crossbeta-β2gpi develop titers against mouse untreated β2gpi, demonstrating breaking of tolerance in the mice when immunized with an antigen that comprises crossbeta structure.

FIG. 9. Detection of amyloid-like misfolded protein conformation comprising crossbeta structure in antigens.

Crossbeta structure is detected by assessing enhancement of ThT fluorescence upon contacting the amyloid-specific dye with an antigen in solution, and by measuring plasmin activity upon activation of tPA, a serine protease that binds to and is activated by crossbeta structure, in a chromogenic plasmin substrate conversion assay. A. tPA/plasminogen activation by 100 μg/ml OVA or DOVA (Misfolding Method I), compared to buffer control. B. ThT fluorescence assay with 100 μg/ml OVA or DOVA. C. ThT fluorescence with tenfold diluted untreated H5 stock (lot 1 210406CS, 236 μg/ml in PBS). D. tPA activation with H5 stock solution (lot 1 210406CS, 236 μg/ml in PBS), showing the presence of a fraction of H5 molecules with crossbeta structure conformation. E. ThT fluorescence with untreated H5, thermal misfolded (H5+OVA) (Method I) and thermal misfolded H5−OVA (conjugated with glutaraldehyde/NaBH₄; Method III), all at 14 μg/ml (lot 2 fraction X 250506CS, 140 μg/ml in PBS). F. tPA activating properties of 12 μg/ml recombinant H7 purchased from Protein Sciences Corp., indicative for the presence of crossbeta structure. Positive control: amyloid γ-globulins. G. Recombinant H7 purchased from Protein Sciences Corp. at 60 μg/ml enhances ThT fluorescence to some extent, indicative for the presence of protein molecules with crossbeta structure. H. ThT fluorescence assay with in-house produced recombinant native H7, thermal misfolded mixture of H7 and OVA, and thermal misfolded H7−OVA conjugate, obtained through EDC/NHS coupling. H7 stock solutions were diluted tenfold. I. Enhancement of tPA/plasminogen activity upon introduction of 1 μg/ml misfolded H7 (lot 2 05-2006CS). ‘(H7+OVA)’ refers to thermal misfolded H7 with added OVA, ‘H7−OVA’ refers to thermal misfolded H7 after coupling of H7 to OVA using EDC/NHS. J. ThT fluorescence upon contacting 10-fold diluted E2 preparations with a DOVA concentration of 100 μg/ml. Free E2 from HEK293E cells was either misfolded directly using thermal cycling, or first conjugated to OVA using EDC/NHS, before misfolding.

K. The enhancement of tPA activity is determined with various CL3 peptide preparations at 100 μg/ml. L. In the ThT fluorescence enhancement assay, concentrations of sample 1, 2, 5 and 6 was 136 μg/ml, whereas samples 3 and 4 were tested at 100 μg/ml.

FIG. 10. Antigens used for avian flu H₅N₁ challenge experiments and CSFV challenge experiments.

A. ThT fluorescence of H5 preparations at 24 μg/ml. For Sample 2, H5 was mixed with OVA before misfolding using thermal cycling was applied. H5−OVA-1, H5 conjugated to OVA using EDC/NHS coupling; H5−OVA-2, H5 conjugated to OVA using glutaraldehyde/NaBH₄ coupling. B. tPA/plasminogen activity assay with native E2 and two misfolded E2 preparations. E2 preparations used for a first vaccination of pigs with untreated E2 or various amyloid-like misfolded forms. E2 lot 1 200406RS at 285 μg/ml in PBS, expressed in Sf1 cells, was used for the immunization. In the assay, the E2 concentration was 20 μg/ml. C. ThT fluorescence of E2 preparations used for vaccine formulation. The misfolded E2 was obtained by thermal cycling from 30 to 85° C. and back. D. In a tPA/plasminogen activation assay, freshly dissolved CL3 peptide and 65° C.-incubated peptide are most potent tPA activators, whereas incubations at room temperature or at 37° C. result in a lower activating crossbeta structure content. Peptide concentration in the assay was 200 μg/ml.

FIG. 11. Determination of the crossbeta structure content in a cocktail vaccine comprising E2, CL3, H5, H7 and ovalbumin.

A. tPA/plasminogen activation is determined with 20-fold diluted cocktail vaccine solutions a-f, resulting in approximately 5 and 0.5 μg/ml total antigen concentration for groups a-c and d-f, respectively. B. Congo red fluorescence assay with 10-fold diluted antigen solutions a-f. Positive control was amyloid-B. C. ThT fluorescence assay with 10-fold diluted antigen solutions a-f Positive control: Aβ.

FIG. 12. Anti-antigen antibody titers in mouse sera at day 28 post-vaccination with crossbeta-adjuvated cocktail of antigens.

A. When mice in group a are vaccinated once with 10 μg untreated recombinant H7 (Protein Sciences Corp.) per animal, anti-H7 antibody titers are developed. B. Titers against E2 antigen of the Cedi-Diagnostics CSFV kit. After one vaccination, mice that received 5 or 10 μg crossbeta-adjuvated E2, expressed in HEK293E cells, developed a titer. C. Titer determination with coated DOVA antigen. D. Titer determination with coated untreated OVA.

FIG. 13: Survival of mice after vaccination with placebo (water, T01), vaccination with subunit vaccine H5 adjuvated with crossbeta structure (groep T03) or vaccinated with inactivated influenza virus (H5N9) (group T10) and challenge with 20 LD₅₀ as described in materials and methods.

A. Anti-H5 titers in pooled mouse sera at day 33 post-vaccination with placebo, untreated H5, commercial H5N9 vaccine or crossbeta-adjuvated H5 were assessed using recombinant H5 from a different source than the antigen used for vaccination, as the antigen in the ELISA. B. Anti-H7 titers in pooled mouse sera at day 33 post-vaccination with placebo, untreated H7, Specol-adjuvated H7 or crossbeta-adjuvated H7 were assessed in an ELISA. C. With pre-immune mouse sera and sera obtained at day 33 post-vaccination with placebo, untreated H5 or crossbeta-adjuvated H5, titers against DOVA were assessed. DOVA was part of the vaccine formulations for groups 3, 4 and 5. D. Survival of mice vaccinated with placebo (T01), crossbeta-H5 antigen (T03) or inactivated virus H5N9 vaccine (T10), after challenge with H5N1 virus. E. Titers determined with individual mouse sera of T03 obtained at day 33 post-vaccination with crossbeta-adjuvated H5. F. Titers determined with individual mouse sera of T10 obtained at day 33 post-vaccination with inactivated H5N9 virus.

FIG. 14: Survival at day 14 of pigs that are vaccinated with E2 adjuvated with crossbeta structure or DOE-adjuvated E2 and challenged with 200 LD₅₀ Classical Swine Fever Virus strain Brescia 456610.

Survival of pigs after challenge with CSFV. Group T03 was vaccinated with crossbeta-E2, as well as pig #143 from Group T05. Group T06 was vaccinated with water-oil emulsion-adjuvated (DOE) E2.

FIG. 15: Auto-immune OVA-antibodies formed upon vaccination with crossbeta-structure comprising vaccines.

Antibodies in sera collected from chicken immunized with vaccines described in the text were determined by ELISA. OVA with amyloid-like properties was used as the antigen in the ELISA.

### REFERENCE LIST

### Except Examples 7-14

1. Kranenburg, O. et al. Tissue-type plasminogen activator is a multiligand cross-beta structure receptor. Curr. Biol. 12, 1833-1839 (2002).
2. Landman, W. J. Amyloid arthropathy in chickens. Vet. Q. 21, 78-82 (1999).
3. Bouma, B. et al. Glycation induces formation of amyloid cross-beta structure in albumin. J. Biol. Chem. 278, 41810-41819 (2003).
4. Hoppener, J. W., Ahren, B. & Lips, C. J. Islet amyloid and type 2 diabetes mellitus. N. Engl. J. Med. 343, 411-419 (2000).
5. Hoppener, J. W. et al. Extensive islet amyloid formation is induced by development of Type II diabetes mellitus and contributes to its progression: pathogenesis of diabetes in a mouse model. Diabetologia 42, 427-434 (1999).
6. de Laat, B., Derksen, R. H., Urbanus, R. T. & de Groot, P. G. IgG antibodies that recognize epitope Gly40-Arg43 in domain I of {beta}2-glycoprotein I cause LAC and their presence correlates strongly with thrombosis. Blood., (2004).
7. Horbach, D. A., van Oort, E., Donders, R. C., Derksen, R. H. & de Groot, P. G. Lupus anticoagulant is the strongest risk factor for both venous and arterial thrombosis in patients with systemic lupus erythematosus. Comparison between different assays for the detection of anitiphospholipid antibodies. Thromb. Haemost. 76, 916-924 (1996).
8. Horbach, D. A., van Oort, E., Tempelman, M. J., Derksen, R. H. & de Groot, P. G. The prevalence of a non-phospholipid-binding form of beta2-glycoprotein I in human plasma—consequences for the development of anti-beta2-glycoprotein I antibodies. Thromb. Haemost. 80, 791-797 (1998).
9. de Laat, H. B., Derksen, R. H., Urbanus, R. T., Roest, M. & de Groot, P. G. beta2-glycoprotein I-dependent lupus anticoagulant highly correlates with thrombosis in the antiphospholipid syndrome. Blood 104, 3598-3602 (2004).
10. Brandenburg, K., Koch, M. H. & Seydel, U. Biophysical characterisation of lysozyme binding to LPS Re and lipid A. Eur. J. Biochem. 258, 686-695 (1998).
11. Takada, K., Ohno, N. & Yadomae, T. Detoxification of lipopolysaccharide (LPS) by egg white lysozyme. FEMS Immunol. Med. Microbiol. 9, 255-263 (1994).
12. Morrison, D. C. & Cochrane, C. G. Direct evidence for Hageman factor (factor XII) activation by bacterial lipopolysaccharides (endotoxins). J. Exp. Med. 140, 797-811 (1974).
13. Gebbink, M. F., Claessen, D., Bouma, B., Dijkhuizen, L. & Wosten, H. A. Amyloids—a functional coat for microorganisms. Nat. Rev. Microbiol. 3, 333-341 (2005).
14. Matsuura, E., Igarashi, Y., Yasuda, T., Triplett, D. A. & Koike, T. Anticardiolipin antibodies recognize beta 2-glycoprotein I structure altered by interacting with an oxygen modified solid phase surface. J. Exp. Med. 179, 457-462 (1994).
15. Subang, R. et al. Phospholipid-bound beta 2-glycoprotein I induces the production of anti-phospholipid antibodies. J. Autoimmun. 15, 21-32 (2000).
16. Levine, J. S., Subang, R., Koh, J. S. & Rauch, J. Induction of anti-phospholipid autoantibodies by beta2-glycoprotein I bound to apoptotic thymocytes. J. Autoimmun. 11, 413-424 (1998).
17. Matzinger, P. An innate sense of danger. Ann. N. Y. Acad. Sci. 961:341-2., 341-342 (2002).
18. Bouma, B. et al. Adhesion mechanism of human beta(2)-glycoprotein I to phospholipids based on its crystal structure. EMBO J. 18, 5166-5174 (1999).

19. Jurgens, G. et al. Investigation into the interaction of recombinant human serum albumin with Re-lipopolysaccharide and lipid A. J. Endotoxin. Res. 8, 115-126 (2002).

20. Butovsky, O., Talpalar, A. E., Ben Yaakov, K. & Schwartz, M. Activation of microglia by aggregated beta-amyloid or lipopolysaccharide impairs MHC-II expression and renders them cytotoxic whereas IFN-gamma and IL-4 render them protective. Mol. Cell. Neurosci., (2005).
21. Fassbender, K. et al. The LPS receptor (CD14) links innate immunity with Alzheimer's disease. FASEB J. 18, 203-205 (2004).
22. Liu, Y. et al. LPS receptor (CD14): a receptor for phagocytosis of Alzheimer's amyloid peptide. Brain., (2005).
23. Lutters, B. C., Meijers, J. C., Derksen, R. H., Arnout, J. & de Groot, P. G. Dimers of beta 2-glycoprotein I mimic the in vitro effects of beta 2-glycoprotein I-anti-beta 2-glycoprotein I antibody complexes. J. Biol. Chem. 276, 3060-3067 (2001).
24. O'Nuallain, B. & Wetzel, R. Conformational Abs recognizing a generic amyloid fibril epitope. Proc. Natl. Acad. Sci. U.S. A 99, 1485-1490 (2002).
25. Kayed, R. et al. Common structure of soluble amyloid oligomers implies common mechanism of pathogenesis. Science 300, 486-489 (2003).

### REFERENCE LIST FOR EXAMPLES 7-14

(1) Luijkx T A, van Dijken H, Hamstra H J et al. Relative immunogenicity of PorA subtypes in a multivalent Neisseria meningitidis vaccine is not dependent on presentation form. Infect Immun. 2003; 71:6367-6371.
(2) Subang R, Levine J S, Janoff A S et al. Phospholipid-bound beta 2-glycoprotein I induces the production of anti-phospholipid antibodies. J. Autoimmun. 2000; 15:21-32.
(3) Wensvoort G, Terpstra C, Boonstra J, Bloemraad M, Van Zaane D. Production of monoclonal antibodies against swine fever virus and their use in laboratory diagnosis. Vet Microbiol. 1986; 12:101-108.
(4) Vaughn J L, Goodwin R H, Tompkins G J, McCawley P. The establishment of two cell lines from the insect Spodoptera frugiperda (Lepidoptera; Noctuidae). In Vitro. 1977; 13:213-217.
(5) Hulst M M, Westra D F, Wensvoort G, Moormann R J. Glycoprotein E1 of hog cholera virus expressed in insect cells protects swine from hog cholera. J. Virol. 1993; 67:5435-5442.
(6) Treanor J J, Campbell J D, Zangwill K M, Rowe T, Wolff M. Safety and immunogenicity of an inactivated subvirion influenza A (H₅N₁) vaccine. N Engl J. Med. 2006; 354:1343-1351.
(7) Poland G A. Vaccines against avian influenza—a race against time. N Engl J. Med. 2006; 354:1411-1413.
(8) Treanor J J, Schiff G M, Couch R B et al. Dose-related safety and immunogenicity of a trivalent baculovirus-expressed influenza-virus hemagglutinin vaccine in elderly adults. J Infect Dis. 2006; 193:1223-1228.
(9) van Rijn P A, Bossers A, Wensvoort G, Moormann R J. Classical swine fever virus (CSFV) envelope glycoprotein E2 containing one structural antigenic unit protects pigs from lethal CSFV challenge. J Gen Virol. 1996; 77:2737-2745.
(10) Bouma A, de Smit A J, de Kluijver E P, Terpstra C, Moormann R J. Efficacy and stability of a subunit vaccine based on glycoprotein E2 of classical swine fever virus. Vet Microbiol. 1999; 66:101-114.
(11) Wensvoort G, Terpstra C, de Kluijver E P, Kragten C, Warnaar J C. Antigenic differentiation of pestivirus strains with monoclonal antibodies against hog cholera virus. Vet Microbiol. 1989; 21:9-20.
(12) Terpstra C, Bloemraad M, Gielkens A L. The neutralizing peroxidase-linked assay for detection of antibody against swine fever virus. Vet Microbiol. 1984; 9:113-120.

TABLE 1
cross-β structure binding compounds

| Congo red | Chrysamine G | Thioflavin T |
|---|---|---|
| 2-(4′-(methylamino)phenyl)-6- | Any other | Glycosaminoglycans |
| methylbenzothiaziole | amyloid-binding | |
| | dye/chemical | |
| Thioflavin S | Styryl dyes | BTA-1 |
| Poly(thiophene acetic acid) | conjugated | |
| | polyeclectrolyte | |
| | PTAA-Li | |

TABLE 2
proteins that are capable of specifically binding to and/or interacting with
misfolded proteins and/or with proteins comprising cross-β structure

| Tissue-type plasminogen | Finger domain(s) of tPA, factor | |
|---|---|---|
| activator | XII, fibronectin, HGFA | Apolipoprotein E |
| Factor XII | Plasmin(ogen) | Matrix metalloprotease-1 |
| Fibronectin | 75 kD-neurotrophin receptor | Matrix metalloprotease-2 |
| | (p75NTR) | |
| Hepatocyte growth factor | α2-macroglobulin | Matrix metalloprotease-3 |
| activator | | |
| Serum amyloid P component | High molecular weight | Monoclonal antibody |
| | kininogen | 2C11(F8A6)^{‡} |
| C1q | Cathepsin K | Monoclonal antibody 4A6(A7)^{‡} |
| CD36 | Matrix metalloprotease 9 | Monoclonal antibody 2E2(B3)^{‡} |
| Receptor for advanced | Haem oxygenase-1 | Monoclonal antibody 7H1(C6)^{‡} |
| glycation endproducts | | |
| Scavenger receptor-A | low-density lipoprotein | Monoclonal antibody 7H2(H2)^{‡} |
| | receptor-related protein (LRP, | |
| | CD91) | |
| Scavenger receptor-B | DnaK | Monoclonal antibody 7H9(B9)^{‡} |
| ER chaperone Erp57 | GroEL | Monoclonal antibody 8F2(G7)^{‡} |
| Calreticulin | VEGF165 | Monoclonal antibody 4F4^{‡} |
| Monoclonal conformational | Monoclonal conformational | Amyloid oligomer specific |
| antibody WO1 (ref. | antibody WO2 (ref. (O'Nuallain | antibody (ref. (Kayed et al., |
| (O'Nuallain and Wetzel, 2002)) | and Wetzel, 2002)) | 2003)) |
| formyl peptide receptor-like 1 | α(6)β(1)-integrin | CD47 |
| Rabbit anti-albumin-AGE | CD40 | apo A-I belonging to small |
| antibody, Aβ-purified^{a)} | | high-density lipoproteins |
| apoJ/clusterin | 10 times molar excess PPACK, | CD40-ligand |
| | 10 mM εACA, (100 pM - 500 nM) | |
| | tPA²⁾ | |
| macrophage scavenger | broad spectrum (human) | BiP/grp78 |
| receptor CD163 | immunoglobulin G (IgG) | |
| | antibodies (IgIV, IVIg) | |
| Erdj3 | haptoglobin | |

^{‡}Monoclonal antibodies developed in collaboration with the ABC-Hybridoma Facility, Utrecht University, Utrecht, The Netherlands.
^{a)}Antigen albumin-AGE and ligand Aβ were send in to Davids Biotechnologie (Regensburg, Germany); a rabbit was immunized with albumin-AGE, antibodies against a structural epitope were affinity purified using a column with immobilized Aβ.
²⁾PPACK is Phe-Pro-Arg-chloromethylketone (SEQ-ID 8), εACA is ε-amino caproic acid, tPA is tissue-type plasminogen activator

TABLE 3
Proteins involved in the “Crossbeta structure pathway”

| | | |
|---|---|---|
| Monoclonal antibody 4B5 | Heat shock protein 27 | Heat shock protein 40 |
| Monoclonal antibody 3H7^{‡} | Nod2 (=CARD15) | Heat shock protein 70 |
| FEEL-1 | Pentraxin-3 | HDT1 |
| LOX-1 | Serum amyloid A proteins | GroES |
| MD2 | Stabilin-1 | Heat shock protein 90 |
| FEEL-2 | Stabilin-2 | CD36 and LIMPII analogous-I |
| | | (CLA-1) |
| Low Density Lipoprotein | LPS binding protein | CD14 |
| C reactive protein | CD45 | Orosomucoid |
| Integrins | alpha-1 antitrypsin | apo A-IV-Transthyretin complex |
| Albumin | Alpha-1 acid glycoprotein | β2-glycoprotein I |
| Lysozyme | Lactoferrin | Megalin |
| Tamm-Horsfall protein | Apolipoprotein E3 | Apolipoprotein E4 |
| Toll-like receptors | Complement receptor CD11b/CD18 (Mac-1, | CD11d/CD18 (subunit aD) |
| | CR3) | |
| CD11b2 | CD11a/CD18 (LFA-1, subunit aL) | CD11c/CD18 (CR4, subunit aX) |
| Von Willebrand factor | Myosin | Agrin |
| Perlecan | Chaperone60 | b2 integrin subunit |
| proteins that act in the unfolded | proteins that act in the endoplasmic reticulum | Macrophage receptor with |
| protein response (UPR) pathway of | stress response (ESR) pathway of prokaryotic | collagenous structure (MARCO) |
| the endoplasmic reticulum (ER) of | and eukaryotic cells | |
| prokaryotic and eukaryotic cells | | |
| 20S | Chaperone 16 family members | HSC73 |
| HSC70 | translocation channel protein Sec61p | 26S proteasome |
| 19S cap of the proteasome (PA700) | UDP-glucose:glycoprotein glucosyl transferase | carboxy-terminus of |
| | (UGGT) | CHAPERONE70-interacting |
| | | protein (CHIP) |
| Pattern Recognition Receptors | Derlin-1 | Calnexin |
| Bcl-2 asociated athanogene (Bag-1) | GRP94 | Endoplasmic reticulum p72 |
| (broad spectrum) (human) | proteins that act in the endoplasmic reticulum | The (very) low density lipoprotein |
| immunoglobulin M (IgM) antibodies | associated degradation system (ERAD) | receptor family |
| Fc receptor | | |

^{‡}Monoclonal antibodies developed in collaboration with the ABC-Hybridoma Facility, Utrecht University, Utrecht, The Netherlands.

TABLE 4
ELISA with mice of group A, alum-adjuvated PorA

Neisseria subtype

| | P1.5-2.10 | | | |
|---|---|---|---|---|
| | titer | | | |
| Mouse # | Day 0 | Day 14 | Day 28 | Day 42 |
| 1 | 50 | 50 | 1788 | 22612 |
| 2 | 50 | 485 | 2545 | 11256 |
| 3 | 50 | 50 | 368 | 1733 |
| 4 | 50 | 50 | 1150 | 14785 |
| 5 | 50 | 50 | 288 | 16255 |
| Average | 50 | 137 | 1228 | 13328 |
| Stand. dev. | 0 | 195 | 959 | 7675 |

TABLE 5
ELISA with mice of group A, alum-adjuvated PorA

Neisseria subtype
P1.12-1.13
titer

| Mouse # | Day 0 | Day 14 | Day 28 | Day 42 |
|---|---|---|---|---|
| 1 | 50 | 50 | 50 | 491 |
| 2 | 50 | 50 | 50 | 2228 |
| 3 | 50 | 50 | 360 | 558 |
| 4 | 50 | 50 | 50 | 426 |
| 5 | 50 | 271 | 50 | 1086 |
| Average | 50 | 94 | 112 | 958 |
| Stand. dev. | 0 | 99 | 139 | 757 |

TABLE 6
ELISA with mice of group A, alum-adjuvated PorA

Neisseria subtype
P1.7-2.4
titer

| Mouse # | Day 0 | Day 14 | Day 28 | Day 42 |
|---|---|---|---|---|
| 1 | 50 | 50 | 50 | 1561 |
| 2 | 50 | 50 | 3363 | 29572 |
| 3 | 50 | 50 | 393 | 59201 |
| 4 | 50 | 50 | 260 | 40023 |
| 5 | 50 | 50 | 221 | 7418 |
| Average | 50 | 50 | 857 | 27555 |
| Stand. dev. | 0 | 0 | 1406 | 23676 |

TABLE 7
ELISA with mice of group B, alum-adjuvated PorA

Neisseria subtype
P1.5-2.10
titer

| Mouse # | Day 0 | Day 14 | Day 28 | Day 42 |
|---|---|---|---|---|
| 1 | 50 | 50 | 168 | 9457 |
| 2 | 50 | 569 | 2129 | 30371 |
| 3 | 50 | 177 | 50 | 27580 |
| 4 | 50 | 50 | 200 | 177000 |
| 5 | 50 | 50 | 363 | 10430 |
| Average | 50 | 179 | 582 | 50968 |
| Stand. dev. | 0 | 225 | 872 | 71102 |

TABLE 8
ELISA with mice of group B, alum-adjuvated PorA

Neisseria subtype
P1.12-1.13
titer

| Mouse # | Day 0 | Day 14 | Day 28 | Day 42 |
|---|---|---|---|---|
| 1 | 50 | 50 | 322 | 50 |
| 2 | 50 | 50 | 50 | 50 |
| 3 | 50 | 50 | 50 | 50 |
| 4 | 50 | 50 | 50 | 50 |
| 5 | 50 | 50 | 50 | 50 |
| Average | 50 | 50 | 104 | 50 |
| Stand. dev. | 0 | 0 | 122 | 0 |

TABLE 9
ELISA with mice of group B, alum-adjuvated PorA

Neisseria subtype
P1.7-2.4
titer

| Mouse # | Day 0 | Day 14 | Day 28 | Day 42 |
|---|---|---|---|---|
| 1 | 50 | 50 | 50 | 3140 |
| 2 | 50 | 50 | 1454 | 9334 |
| 3 | 50 | 50 | 1086 | 604 |
| 4 | 50 | 50 | 50 | 256 |
| 5 | 50 | 50 | 433 | 51728 |
| Average | 50 | 50 | 615 | 13012 |
| Stand. dev. | 0 | 0 | 632 | 21946 |

TABLE 10
ELISA with mice of group C, alum-adjuvated PorA

Neisseria subtype
P1.5-2.10
titer

| Mouse # | Day 0 | Day 14 | Day 28 | Day 42 |
|---|---|---|---|---|
| 1 | 50 | 50 | 50 | 50 |
| 2 | 50 | 50 | 50 | 50 |
| 3 | 50 | 50 | 50 | 50 |
| 4 | 50 | 50 | 50 | 50 |
| 5 | 50 | 50 | 50 | 50 |
| Average | 50 | 50 | 50 | 50 |
| Stand. dev. | 0 | 0 | 0 | 0 |

TABLE 11
ELISA with mice of group C, alum-adjuvated PorA

Neisseria subtype
P1.12-1.13
titer

| Mouse # | Day 0 | Day 14 | Day 32 | Day 42 |
|---|---|---|---|---|
| 1 | 50 | 50 | 50 | 50 |
| 2 | 50 | 50 | 50 | 50 |
| 3 | 50 | 50 | 50 | 50 |
| 4 | 50 | 50 | 50 | 50 |
| 5 | 50 | 50 | 50 | 50 |
| Average | 50 | 50 | 50 | 50 |
| Stand. dev. | 0 | 0 | 0 | 0 |

TABLE 12
ELISA with mice of group C, alum-adjuvated PorA

Neisseria subtype
P1.7-2.4
titer

| Mouse # | Day 0 | Day 14 | Day 28 | Day 42 |
|---|---|---|---|---|
| 1 | 50 | 50 | 50 | 50 |
| 2 | 50 | 50 | 50 | 50 |
| 3 | 50 | 50 | 50 | 50 |
| 4 | 50 | 50 | 50 | 50 |
| 5 | 50 | 50 | 50 | 50 |
| Average | 50 | 50 | 50 | 50 |
| Stand. dev. | 0 | 0 | 0 | 0 |

TABLE 13
ELISA with mice of group D, alum-adjuvated PorA

Neisseria subtype
P1.5-2.10
titer

| Mouse # | Day 0 | Day 14 | Day 28 | Day 42 |
|---|---|---|---|---|
| 1 | 50 | 379 | 1331 | 34857 |
| 2 | 50 | 50 | 809 | 8932 |
| 3 | 50 | 176 | 111 | 1397 |
| 4 | 50 | 50 | 422 | 243 |
| 5 | 50 | 50 | 553 | 22067 |
| Average | 50 | 141 | 645 | 13499 |
| Stand. dev. | 0 | 144 | 458 | 14770 |

TABLE 14
ELISA with mice of group D, alum-adjuvated PorA

Neisseria subtype
P1.12-1.13
titer

| Mouse # | Day 0 | Day 14 | Day 28 | Day 42 |
|---|---|---|---|---|
| 1 | 50 | 50 | 50 | 690 |
| 2 | 50 | 50 | 50 | 7713 |
| 3 | 50 | 50 | 488 | 50 |
| 4 | 50 | 50 | 50 | 50 |
| 5 | 50 | 50 | 50 | 1104 |
| Average | 50 | 50 | 138 | 1921 |
| Stand. dev. | 0 | 0 | 196 | 3268 |

TABLE 15
A with mice of group D, alum-adjuvated PorA

Neisseria subtype
P1.7-2.4
titer

| # | Day 0 | Day 14 | Day 28 | Day 42 |
|---|---|---|---|---|
| 1 | 50 | 50 | 50 | 1285 |
| 2 | 50 | 123 | 780 | 12423 |
| 3 | 50 | 211 | 601 | 46043 |
| 4 | 50 | 227 | 50 | 26469 |
| 5 | 50 | 50 | 50 | 975 |
| Average | 50 | 132 | 306 | 17439 |
| Stand. dev. | 0 | 85 | 356 | 19085 |

TABLE 16
ELISA with mice of group E, alum-adjuvated PorA

Neisseria subtype
P1.5-2.10
titer

| Mouse # | Day 0 | Day 14 | Day 28 | Day 42 |
|---|---|---|---|---|
| 1 | 50 | 50 | 319 | 13565 |
| 2 | 50 | 160 | 1285 | 22402 |
| 3 | 50 | 1691 | 1530 | 4532 |
| 4 | 50 | 50 | 50 | 288 |
| 5 | 50 | 266 | 389 | 4150 |
| Average | 50 | 443 | 715 | 8987 |
| Stand. dev. | 0 | 703 | 651 | 8942 |

TABLE 17
ELISA with mice of group E, alum-adjuvated PorA

Neisseria subtype
P1.12-1.13
titer

| Mouse # | Day 0 | Day 14 | Day 28 | Day 42 |
|---|---|---|---|---|
| 1 | 50 | 50 | 116 | 50 |
| 2 | 50 | 50 | 50 | 50 |
| 3 | 50 | 50 | 50 | 50 |
| 4 | 50 | 50 | 50 | 50 |
| 5 | 50 | 50 | 50 | 756 |
| Average | 50 | 50 | 63 | 191 |
| Stand. dev. | 0 | 0 | 30 | 316 |

TABLE 18
ELISA with mice of group E, alum-adjuvated PorA

Neisseria subtype
P1.7-2.4
titer

| Mouse # | Day 0 | Day 14 | Day 28 | Day 42 |
|---|---|---|---|---|
| 1 | 50 | 50 | 50 | 499 |
| 2 | 50 | 50 | 1361 | 24390 |
| 3 | 50 | 112 | 244 | 4113 |
| 4 | 50 | 50 | 50 | 50 |
| 5 | 50 | 50 | 417 | 1473 |
| Average | 50 | 62 | 424 | 6105 |
| Stand. dev. | 0 | 28 | 545 | 10342 |

TABLE 19
SBA with mice of group A, alum-adjuvated PorA

| | Neisseria subtype | | | | | |
|---|---|---|---|---|---|---|
| | P1.5-2.10 | | P1.12-1.13 | | P1.7-2.4 | |
| | titer | | titer | | titer | |
| Mouse # | Day 28 | Day 42 | Day 28 | Day 42 | Day 28 | Day 42 |
| 1 | 160 | 320 | 5 | 5 | 5 | 5 |
| 2 | 5 | 160 | 5 | 5 | 5 | 1280 |
| 3 | 5 | 5 | 5 | 5 | 5 | 1280 |
| 4 | 160 | 1280 | 5 | 5 | 5 | 5 |
| 5 | 5 | 1280 | 5 | 5 | 5 | 5 |
| Average | 67 | 609 | 5 | 5 | 5 | 515 |
| Stand. dev. | 76 | 557 | 0 | 0 | 0 | 625 |

TABLE 20
SBA with mice of group B, alum-adjuvated PorA

| | Neisseria subtype | | | | | |
|---|---|---|---|---|---|---|
| | P1.5-2.10 | | P1.12-1.13 | | P1.7-2.4 | |
| | titer | | titer | | titer | |
| Mouse # | Day 28 | Day 42 | Day 28 | Day 42 | Day 28 | Day 42 |
| 1 | 5 | 1280 | 5 | 640 | 5 | 10 |
| 2 | 1280 | 1280 | 5 | 5 | 10 | 20 |
| 3 | 5 | 1280 | 5 | 5 | 5 | 5 |
| 4 | 5 | 320 | 5 | 5 | 5 | 5 |
| 5 | 5 | 640 | 5 | 5 | 5 | 1280 |
| Average | 260 | 960 | 5 | 132 | 6 | 264 |
| Stand. dev. | 510 | 405 | 0 | 254 | 2 | 508 |

TABLE 21
SBA with mice of group C, alum-adjuvated PorA

| | Neisseria subtype | | | | | |
|---|---|---|---|---|---|---|
| | P1.5-2.10 | | P1.12-1.13 | | P1.7-2.4 | |
| | titer | | titer | | titer | |
| Mouse # | Day 28 | Day 42 | Day 28 | Day 42 | Day 28 | Day 42 |
| 1 | 5 | 5 | 5 | 5 | 5 | 5 |
| 2 | 5 | 5 | 5 | 5 | 5 | 5 |
| 3 | 5 | 5 | 5 | 5 | 5 | 5 |
| 4 | 5 | 5 | 5 | 5 | 5 | 5 |
| 5 | 5 | 5 | 5 | 5 | 5 | 5 |
| Average | 5 | 5 | 5 | 5 | 5 | 5 |
| Stand. dev. | 0 | 0 | 0 | 0 | 0 | 0 |

TABLE 22
SBA with mice of group D, alum-adjuvated PorA

| | Neisseria subtype | | | | | |
|---|---|---|---|---|---|---|
| | P1.5-2.10 | | P1.12-1.13 | | P1.7-2.4 | |
| | titer | | titer | | titer | |
| Mouse # | Day 28 | Day 42 | Day 28 | Day 42 | Day 28 | Day 42 |
| 1 | 40 | 1280 | 5 | 5 | 5 | 5 |
| 2 | 5 | 5 | 5 | 5 | 5 | 5 |
| 3 | 5 | 640 | 5 | 5 | 5 | 160 |
| 4 | 5 | 5 | 5 | 5 | 5 | 5 |
| 5 | 20 | 1280 | 5 | 5 | 5 | 5 |
| Average | 15 | 642 | 5 | 5 | 5 | 36 |
| Stand. dev. | 14 | 570 | 0 | 0 | 0 | 62 |

TABLE 23
SBA with mice of group E, alum-adjuvated PorA

| | Neisseria subtype | | | | | |
|---|---|---|---|---|---|---|
| | P1.5-2.10 | | P1.12-1.13 | | P1.7-2.4 | |
| | titer | | titer | | titer | |
| Mouse # | Day 28 | Day 42 | Day 28 | Day 42 | Day 28 | Day 42 |
| 1 | 5 | 5 | 5 | 5 | 5 | 5 |
| 2 | 20 | 1280 | 5 | 5 | 5 | 320 |
| 3 | 80 | 320 | 5 | 5 | 5 | 160 |
| 4 | 5 | 10 | 5 | 5 | 5 | 5 |
| 5 | 10 | 80 | 5 | 40 | 5 | 5 |
| Average | 24 | 339 | 5 | 12 | 5 | 99 |
| Stand. dev. | 29 | 484 | 0 | 14 | 0 | 126 |

TABLE 24
Comparison of anti-PorA antibody titers and
bactericidal antibody titers

| | | Titers against PorA subtypes^{∫} | | | |
|---|---|---|---|---|---|
| group^{†} | Mouse # | P1.5-2,10 | P1.12-1,13 | P1.7-2,4 | Assay^{‡} |
| A | 1 | + | − | − | ELISA |
| A | 1 | + | − | − | SBA |
| A | 2 | + | − | + | ELISA |
| A | 2 | +/− | − | +++ | SBA |
| AA | 33 | −− | −− | 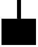 | ELISASBA |
| A | 4 | + | − | + | ELISA |
| A | 4 | +++ | − | − | SBA |
| A | 5 | + | − | +/− | ELISA |
| A | 5 | +++ | − | − | SBA |
| BB | 11 | ++++ | 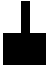 | +/−− | ELISASBA |
| B | 2 | ++ | − | + | ELISA |
| B | 2 | +++ | − | − | SBA |
| B | 3 | ++ | − | − | ELISA |
| B | 3 | +++ | − | − | SBA |
| BB | 44 | ++++++ | −− | 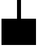 | ELISASBA |
| B | 5 | + | − | ++ | ELISA |
| B | 5 | ++ | − | +++ | SBA |
| D | 1 | ++ | − | − | ELISA |
| D | 1 | +++ | − | − | SBA |
| D | 2 | + | − | + | ELISA |
| D | 2 | − | − | − | SBA |
| DD | 33 | 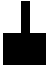 | −− | +/−+/− | ELISASBA |
| D | 4 | − | − | − | ELISA |
| D | 4 | − | − | − | SBA |
| D | 5 | + | − | − | ELISA |
| D | 5 | +++ | − | − | SBA |
| E | 1 | + | − | − | ELISA |
| E | 1 | − | − | − | SBA |
| E | 2 | + | +/− | +/− | ELISA |
| E | 2 | +++ | − | + | SBA |
| E | 3 | +/− | − | + | ELISA |
| E | 3 | + | − | +/− | SBA |
| E | 4 | − | − | + | ELISA |
| E | 4 | − | − | − | SBA |
| E | 5 | +/− | − | − | ELISA |
| E | 5 | +/− | +/− | − | SBA |

^{†}group A, alum-adjuvated PorA; B, non-adjuvated PorA; D, 25% crossbeta-PorA adjuvated; 75% crossbeta-PorA adjuvated.
^{∫}absolute titers are given relative-signs for no titer, + signs for titers. More + signs refers to relatively higher titers.
^{‡}ELISA, anti-PorA antibody ELISA; SBA, serum bactericidal assay. Both assays with individual PorA subtypes.

TABLE 25
Summary of symptoms in animals treated with
Gallimune ® FLU H5N9

| | | Number of mice | |
|---|---|---|---|
| group | Day | with symptoms | Symptom(s) |
| T10 | 22-25 | 8 | Upright hairs |
| T10 | 22-25 | 8 | Sitting with hunchback |
| T10 | 26 | 2 | Upright hairs |
| T10 | 26 | 1 | Sitting with hunchback |
| T10 | 30-41 | 2 | Swelling at inoculation site |

TABLE 26
clinical scores after H5N1 inoculation

| | | Total score per group | | | | | | | | | | | | | | |
|---|---|---|---|---|---|---|---|---|---|---|---|---|---|---|---|---|
| | | Days post infection | | | | | | | | | | | | | | |
| Group/Treatment | | 1 | 2 | 3 | 4 | 5 | 6 | 7 | 8 | 9 | 10 | 11 | 12 | 13 | 14 | # |
| T01 | water | 0 | 0 | 3 | 7 | 7 | 7 | 21 | 21 | 12 | 9 | 9 | 9 | 6 | 3 | 2 |
| T03 | Crossbeta- | 0 | 0 | 7 | 8 | 8 | 8 | 18 | 14 | 12 | 11 | 10 | 5 | 1 | 0 | 8 |
| | H5 | | | | | | | | | | | | | | | |
| | I-III (33%) | | | | | | | | | | | | | | | |
| T10 | Inactivated | 0 | 0 | 3 | 8 | 8 | 8 | 6 | 5 | 3 | 3 | 1 | 1 | 0 | 0 | 8 |
| | avian | | | | | | | | | | | | | | | |
| | influenza | | | | | | | | | | | | | | | |
| | virus (H5N9) | | | | | | | | | | | | | | | |

# Survival number.

TABEL 27
Respiratory symptoms after challenge with H5N1

| | | Number of mice with symptoms | | | | | | | | | | | | | | |
|---|---|---|---|---|---|---|---|---|---|---|---|---|---|---|---|---|
| | | Days post infection | | | | | | | | | | | | | | |
| Group/Treatment | | 1 | 2 | 3 | 4 | 5 | 6 | 7 | 8 | 9 | 10 | 11 | 12 | 13 | 14 | # |
| T01 | water | 0 | 0 | 0 | 0 | 0 | 0 | 7/7 | 7/7 | 4/4 | 3/3 | 3/3 | 3/3 | 2/3 | 2/2 | 2 |
| T03 | Crossbeta- | 0 | 0 | 0 | 0 | 0 | 0 | 5/8 | 4/8 | 3/8 | 3/8 | 3/8 | 1/8 | 0 | 0 | 8 |
| | H5 | | | | | | | | | | | | | | | |
| | I-III (33%) | | | | | | | | | | | | | | | |
| T10 | Inactivated | 0 | 0 | 0 | 0 | 0 | 0 | 0 | 0 | 0 | 0 | 0 | 0 | 0 | 0 | 8 |
| | avian | | | | | | | | | | | | | | | |
| | influenza | | | | | | | | | | | | | | | |
| | virus | | | | | | | | | | | | | | | |
| | (H5N9) | | | | | | | | | | | | | | | |

# Survival number.

TABLE 28
Lethality after H5N1 inoculation

| | | Number of mice per group per day | | | | | | | | | | | | | | | |
|---|---|---|---|---|---|---|---|---|---|---|---|---|---|---|---|---|---|
| | | Days post infection | | | | | | | | | | | | | | | |
| Group/Treatment | | 0 | 1 | 2 | 3 | 4 | 5 | 6 | 7 | 8 | 9 | 10 | 11 | 12 | 13 | 14 | $ |
| T01 | water | 8 | 7 | 7 | 7 | 7 | 7 | 7 | 7 | 7 | 4 | 3 | 3 | 3 | 3 | 2 | 6 |
| T03 | Crossbeta- | 8 | 8 | 8 | 8 | 8 | 8 | 8 | 8 | 8 | 8 | 8 | 8 | 8 | 8 | 8 | 0 |
| | H5 | | | | | | | | | | | | | | | | |
| | I-III (33%) | | | | | | | | | | | | | | | | |
| T10 | Inactivated | 8 | 8 | 8 | 8 | 8 | 8 | 8 | 8 | 8 | 8 | 8 | 8 | 8 | 8 | 8 | 0 |
| | avian | | | | | | | | | | | | | | | | |
| | influenza | | | | | | | | | | | | | | | | |
| | virus | | | | | | | | | | | | | | | | |
| | (H5N9) | | | | | | | | | | | | | | | | |

$ Died/euthanized mice.

TABLE 29
NPLA neutralization titers

| | | | | | | Day | Day | Day | Day | Day | Day |
|---|---|---|---|---|---|---|---|---|---|---|---|
| # | group | Day 0 | Day 2 | Day 5 | Day 9 | 12 | 16 | 19 | 26 | 33 | 42 |
| 114 | T01 | — | — | — | — | — | — | — | — | — | — |
| 115 | T01 | — | — | — | — | 10 | — | — | — | — | — |
| 116 | T01 | — | — | — | — | — | — | — | — | — | — |
| 117 | T01 | — | — | — | — | 15 | — | — | — | — | — |
| 118 | T01 | — | — | — | — | — | — | — | — | — | — |
| 119 | T01 | — | — | — | 10 | — | — | — | 15 | — | — |
| 12- | T02 | — | — | — | — | 10 | — | — | 10 | — | # |
| 121 | T02 | — | — | — | — | 15 | — | — | 20 | — | # |
| 122 | T02 | — | — | — | — | 10 | — | — | 10 | — | # |
| 123 | T02 | — | — | — | — | — | — | — | — | — | # |
| 124 | T02 | — | — | — | — | — | — | — | — | — | # |
| 125 | T02 | — | — | — | — | — | — | — | 20 | 20 | # |
| 126 | T03 | — | 10 | — | — | — | — | — | 15 | 20 | 15 |
| 127 | T03 | — | 10 | — | — | — | — | — | 10 | — | — |
| 128 | T03 | — | — | — | — | — | — | — | 10 | 10 | — |
| 129 | T03 | — | — | — | — | — | — | — | 30 | 20 | 30 |
| 130 | T03 | — | — | — | — | — | — | — | 20 | 0 | 15 |
| 131 | T03 | — | — | — | — | 15 | — | — | 20 | 10 | 15 |
| 132 | T04 | — | — | — | — | — | — | — | 10 | — | # |
| 133 | T04 | — | — | — | 10 | — | — | — | — | — | # |
| 134 | T04 | — | — | — | — | — | — | — | 20 | 20 | # |
| 135 | T04 | — | — | — | — | — | — | — | 10 | — | # |
| 136 | T04 | — | — | — | — | 15 | — | — | 20 | — | # |
| 137 | T04 | — | — | — | — | 10 | — | — | 20 | 20 | # |
| 138 | T05 | — | — | — | — | — | — | — | 10 | 10 | # |
| 139 | T05 | — | 10 | — | — | — | — | — | 15 | 10 | # |
| 140 | T05 | — | — | 15 | — | — | — | — | 15 | — | # |
| 141 | T05 | — | — | — | — | — | — | — | 10 | 10 | # |
| 142 | T05 | — | — | — | — | — | — | — | 20 | — | # |
| 143 | T05 | — | — | — | — | — | — | — | 240 | 60 | 160 |
| 144 | T06 | — | — | — | 10 | 160 | 640 | 3840 | ≧10240 | 40960 | 81920 |
| 145 | T06 | — | — | — | 15 | 120 | 960 | 2560 | ≧10240 | 20480 | 122880 |
| 146 | T06 | — | — | — | 15 | 80 | 80 | 320 | 10240 | 2560 | 10240 |
| 147 | T06 | — | — | — | 15 | 60 | 240 | 1280 | ≧10240 | 20480 | 61440 |
| 148 | T06 | — | — | — | 10 | 30 | 480 | 5120 | ≧10240 | ≧40960 | 61440 |
| 149 | T06 | — | — | — | 15 | 160 | 160 | 640 | 10240 | 10240 | 20480 |

0, titer <10.

TABLE 30
Summary of clinical symptoms

| | | | Digestion | Respiratory | |
|---|---|---|---|---|---|
| group | | Malaise | Problems | Problems | Bleedings |
| T01 | Control | (60%) 76% | (8%) 23% | (8%) 22% (33%) | (20%) 26% (38%) |
| | | (100%) | (38%) | | |
| T03 | E2-crossbeta | (58%) 65% | (13%) 37% | — | (7%) 8% (20%) |
| | adjuvated | (71%) | (60%) | | |

| | | | | |
|---|---|---|---|---|
| Student T-test | 0.092 | 0.078 | 0.006 | 0.002 |
| (P-value) | | | | |

TABLE 31
Clinical scores post challenge

| | | Days post infection | | | | | | | | | | | | | | |
|---|---|---|---|---|---|---|---|---|---|---|---|---|---|---|---|---|
| # | ThX | 0 | 1 | 2 | 3 | 4 | 5 | 6 | 7 | 8 | 9 | 10 | 11 | 12 | 13 | 14 |
| 114 | T01 | — | — | — | — | 1 | 3 | 3 | 3 | 3 | 5 | 5 | 4 | ‡ | | |
| 115 | T01 | 1 | 1 | 1 | 1 | 1 | 4 | 4 | 5 | 5 | 6 | 6 | 4 | 6, ‡ | | |
| 116 | T01 | — | — | — | — | 2 | 2 | 3 | 3 | 3 | 3 | 4 | 4 | 6, ‡ | | |
| 117 | T01 | — | — | — | — | 2 | 2 | 3 | 3 | 5 | 7, | | | | | |
| | | | | | | | | | | | ‡ | | | | | |
| 118 | T01 | — | — | — | — | 2 | 2 | 4 | 3 | 3 | 3 | 5 | 5 | ‡ | | |
| 119 | T01 | — | — | — | — | 2 | 2 | 4 | 4 | 4 | 5 | 5 | 5 | 6, ‡ | | |
| 126 | T03 | — | — | — | — | — | 2 | 5 | 4 | 3 | 3 | 4 | 2 | 4 | 3 | 2 |
| 127 | T03 | — | — | — | — | — | 1 | 3 | 3 | 3 | 4 | 4 | 4 | † | | |
| 128 | T03 | — | — | — | — | — | 2 | 3 | 3 | 4 | 5 | 5 | 2 | 3 | 4 | 5 |
| 129 | T03 | — | — | — | — | — | 1 | 3 | 2 | 2 | 2 | 3 | 2 | 2 | 1 | 1 |
| 130 | T03 | — | — | — | — | — | 1 | 3 | 3 | 2 | 2 | 4 | 2 | 3 | 3 | 3 |
| 131 | T03 | — | — | — | — | — | 2 | 4 | 4 | 4 | 3 | 3 | 4 | 4 | 4 | 4 |
| 143 | T05 | — | — | — | — | — | 2 | 4 | 3 | 4 | 3 | 4 | 4 | 4 | 3 | 2 |
| 144 | T06 | — | — | — | — | — | 1 | 1 | 1 | 1 | 1 | 1 | 1 | 1 | 1 | 1 |
| 145 | T06 | — | — | — | — | — | — | — | — | — | — | — | — | — | — | — |
| 146 | T06 | — | — | — | — | — | — | — | — | — | — | — | — | — | — | — |
| 147 | T06 | — | — | — | — | — | — | — | — | — | — | — | — | — | — | — |
| 148 | T06 | — | — | — | — | — | — | — | — | — | — | — | — | — | — | — |
| 149 | T06 | — | — | — | — | — | — | — | — | — | — | — | — | — | — | — |

† Died;
‡ euthanized.
Numbers indicate number of clinical signs.

TABLE 32
Temperature

| | | Days post infection | | | | | | | | | | | | | | |
|---|---|---|---|---|---|---|---|---|---|---|---|---|---|---|---|---|
| # | group | 1 | 2 | 3 | 4 | 5 | 6 | 7 | 8 | 9 | 10 | 11 | 12 | 13 | 14 | D |
| 114 | T01 | 39.4 | 39.8 | 40.2 | 41.3 | 41.1 | 41.5 | 41.5 | 41.6 | 40.8 | 40.2 | 40.2 | 38.3 | | | 9 |
| 115 | T01 | 39.2 | 39.4 | 39.9 | 41.3 | 41.1 | 43.0 | 42.0 | 41.6 | 41.0 | 41.4 | 40.4 | 40.4 | 39.4 | | 9 |
| 116 | T01 | 39.7 | 39.7 | 40.3 | 40.8 | 41.0 | 41.4 | 40.9 | 41.3 | 41.3 | 40.4 | 40.4 | 39.6 | 38.6 | | 9 |
| 117 | T01 | 39.2 | 39.5 | 40.2 | 41.8 | 41.4 | 42.0 | 43.0 | 41.6 | 41.1 | | | | | | 7 |
| 118 | T01 | 39.6 | 39.8 | 40.3 | 41.8 | 41.2 | 41.5 | 41.7 | 41.7 | 41.1 | 41.3 | 40.5 | 39.7 | | | 9 |
| 119 | T01 | 39.4 | 39.7 | 39.9 | 40.9 | 41.0 | 41.3 | 41.2 | 41.2 | 40.7 | 41.2 | 39.8 | 39.3 | 38.7 | | 7 |
| 126 | T03 | 39.4 | 39.8 | 40.6 | 41.7 | 41.5 | 41.2 | 40.1 | 40.8 | 40.3 | 39.9 | 39.9 | 40.8 | 39.1 | 38.5 | 8 |
| 127 | T03 | 39.6 | 39.7 | 40.0 | 41.3 | 41.3 | 41.8 | 41.6 | 40.0 | 40.9 | 41.2 | 41.1 | | | | 9 |
| 128 | T03 | 39.3 | 39.2 | 40.0 | 41.6 | 41.9 | 40.6 | 43.0 | 41.8 | 41.3 | 40.9 | 41.0 | 40.6 | 40.0 | 40.3 | 12 |
| 129 | T03 | 39.6 | 39.6 | 39.9 | 40.4 | 40.6 | 41.3 | 41.3 | 40.2 | 40.1 | 40.5 | 39.5 | 39.7 | 39.7 | 39.7 | 7 |
| 130 | T03 | 39.6 | 39.2 | 39.5 | 40.9 | 41.3 | 41.3 | 40.2 | 41.2 | 41.3 | 41.3 | 41.3 | 41.1 | 41.3 | 41.2 | 11 |
| 131 | T03 | 39.6 | 39.5 | 40.3 | 41.5 | 41.5 | 41.0 | 41.4 | 40.7 | 40.0 | 40.0 | 39.9 | 39.1 | 39.0 | 39.6 | 8 |
| 143 | T05 | 38.8 | 38.8 | 39.4 | 40.1 | 41.4 | 41.4 | 41.8 | 41.5 | 40.7 | 40.0 | 40.1 | 40.3 | 39.2 | 39.4 | 9 |
| 144 | T06 | 39.6 | 39.1 | 39.0 | 39.2 | 39.8 | 39.0 | 39.2 | 39.4 | 39.6 | 39.1 | 39.2 | 39.2 | 38.7 | 39.0 | 0 |
| 145 | T06 | 39.1 | 39.5 | 39.3 | 39.2 | 39.3 | 39.1 | 39.3 | 39.2 | 39.4 | 39.1 | 39.3 | 39.2 | 38.9 | 39.2 | 0 |
| 146 | T06 | 39.2 | 39.3 | 38.9 | 39.1 | 39.5 | 39.3 | 39.4 | 39.3 | 39.7 | 39.0 | 39.6 | 39.5 | 38.9 | 39.3 | 0 |
| 147 | T06 | 39.3 | 39.0 | 38.9 | 39.0 | 39.5 | 38.9 | 39.1 | 38.9 | 39.5 | 38.7 | 39.0 | 39.0 | 39.1 | 39.0 | 0 |
| 148 | T06 | 39.3 | 39.3 | 39.2 | 39.5 | 39.7 | 39.2 | 39.4 | 39.2 | 39.5 | 39.2 | 39.5 | 39.3 | 39.2 | 39.3 | 0 |
| 149 | T06 | 38.9 | 38.9 | 38.7 | 38.7 | 39.0 | 38.6 | 39.1 | 38.9 | 39.5 | 39.2 | 39.2 | 39.0 | 38.9 | 39.4 | 0 |

In the last column the number of days with fever (>40.0° C.) is given.

TABLE 33
Clinical symptoms scores

| | | | | | | | | | Respiratory | | | | | |
|---|---|---|---|---|---|---|---|---|---|---|---|---|---|---|
| | | | General Malaise | | | Digestion problems | | | Symptoms | | | bleedings | | |
| # | group | Survival | Start | End | Length | Start | End | Length | Start | End | Length | Start | End | Length |
| 114 | T01 | 12 | 4 | 12 | 8 | 10 | 10 | 1 | 9 | 12 | 4 | 9 | 12 | 3 |
| 115 | T01 | 13 | 0 | 12 | 13 | 5 | 12 | 5 | 10 | 13 | 4 | 10 | 13 | 3 |
| 116 | T01 | 13 | 4 | 13 | 10 | 10 | 11 | 2 | 13 | 13 | 1 | 11 | 13 | 3 |
| 117 | T01 | 10 | 4 | 9 | 6 | — | — | — | — | — | — | 8 | 9 | 2 |
| 118 | T01 | 12 | 4 | 12 | 9 | 6 | 12 | 2 | 10 | 11 | 2 | 10 | 12 | 3 |
| 119 | T01 | 13 | 4 | 13 | 10 | 6 | 12 | 5 | 11 | 13 | 3 | 8 | 13 | 5 |
| 126 | T03 | >15 | 5 | 14 | 10 | 6 | 12 | 4 | — | — | — | 11 | 11 | 1 |
| 127 | T03 | 12 | 5 | 11 | 7 | 7 | 11 | 5 | — | — | — | 11 | 11 | 1 |
| 128 | T03 | >15 | 5 | 14 | 10 | 8 | 14 | 6 | — | — | — | 12 | 14 | 3 |
| 129 | T03 | >15 | 5 | 13 | 9 | 9 | 10 | 2 | — | — | — | — | — | — |
| 130 | T03 | >15 | 5 | 14 | 10 | 7 | 14 | 6 | — | — | — | — | — | — |
| 131 | T03 | >15 | 5 | 14 | 10 | 6 | 14 | 9 | — | — | — | 11 | 14 | 2 |
| 143 | T05 | >15 | 5 | 13 | 9 | 6 | 14 | 8 | — | — | — | — | — | — |

Start: the first day a clinical sign of disease is observed.
End: the final day a clinical sign is observed.
Length: the number of days with a clinical sign.
Note:
Not corrected for survival.

TABLE 34
Summary of observed leucopenia en thrombocytopenia
after challenge infection

| group | | | Leucopenia | | | Thrombocytopenia | | |
|---|---|---|---|---|---|---|---|---|
| T01 | Placebo | N = 6* | (2) | 4 | (5) | (5) | 7 | (9) |
| T03 | crossbeta-E2 | N = 6 | (2) | 7 | (10) | (2) | 7 | (10) |
| T06 | E2 + DOE | N = 6 | — | — | — | (1) | 1 | (1) |

| | | |
|---|---|---|
| T03 versus T01 (T-test) | 0.240 | 0.814 |
| T03 versus T06 (T-test) | 0.005 | 0.048 |

*all pigs died or have been euthanized.

### Sequence Identities

### Sequence ID 1

DNA sequence of avian influenza haemagglutinin-5 (H5)

ggatccgatcagatttgcattggttaccatgcaaacaactcgacagagca
ggttgacacaataatggaaaagaatgttactgttacacatgcccaagaca
tactggaaaggacacacaacgggaagctctgcgatctaaatggagtgaag
cctctcattttgagggattgtagtgtagctggatggctcctcggaaaccc
tatgtgtgacgaattcatcaatgtgccggaatggtcttacatagtggaga
aggccagtccagccaatgacctctgttatccagggaatttcaacgactat
gaagaactgaaacacctattgagcagaataaaccattttgagaaaattca
gatcatccccaaaagttcttggtccaatcatgatgcctcatcaggggtga
gctcagcatgtccataccttgggaggtcctcctttttcagaaatgtggta
tggcttatcaaaaagaacagtgcatacccaacaataaagaggagctacaa
taataccaaccaagaagatcttttggtactgtgggggattcaccatccta
aagatgcggcagagcagacaaagctctatcaaaatccaaccacctacatt
tccgttggaacatcaacactgaaccagagattggttccagaaatagctac
tagacccaaagtaaacgggcaaagtggaagaatggagttcttctggacaa
ttttaaagccgaatgatgccatcaatttcgagagtaatggaaatttcatt
gctccagaatatgcatacaaaattgtcaagaaaggggactcaacaattat
gaagagtgaattggaatatggtaactgcaacaccaagtgtcaaactccaa
tgggggcgataaactctagtatgccattccacaacatacaccccctcacc
atcggggaatgccccaaatatgtgaaatcaaacagattagtccttgcgac
tggactcagaaatacccctcaaagagagagaagaagaaaaaagagaggac
tatttggagctatagcaggttttatagagggaggatggcagggaatggta
gatggttggtatgggtaccaccatagcaatgagcaggggagtggatacgc
tgcagacaaagaatccactcaaaaggcaatagatggagtcaccaataagg
tcaactcgatcattaacaaaatgaacactcagtttgaggccgttggaagg
gaatttaataacttggaaaggaggatagagaatttaaacaagaagatgga
agacggattcctagatgtctggacttacaatgctgaacttctggttctca
tggaaaatgagagaactctcgactttcatgactcaaatgtcaagaacctt
tacgacaaggtccgactacagcttagggataatgcaaaggagctgggtaa
tggttgtttcgaattctatcacaaatgtgataatgaatgtatggaaagtg
taaaaaacggaacgtatgactacccgcagtattcagaagaagcaagacta
aacagagaggaaataagtggagtaaaattggaatcaatgggaacatacca
aatactggcggccgc

### Sequence ID 2

Amino acid sequence of avian influenza haemagglutinin-5 (H5)

mrpwtwvllllllicapsyagsdqicigyhannsteqvdtimeknvtvth
aqdilerthngklcdlngvkplilrdcsvagwllgnpmcdefinvpewsy
ivekaspandlcypgnfndyeelkhllsrinhfekiqiipksswsnhdas
sgvssacpylgrssffrnvvwlikknsayptikrsynntnqedllvlwgi
hhpkdaaeqtklyqnpttyisvgtstlnqrlvpeiatrpkvngqsgrmef
fwtilkpndainfesngnfiapeyaykivkkgdstimkseleygncntkc
qtpmgainssmpfhnihpltigecpkyvksnrlvlatglrntpqrerrrk
krglfgaiagfieggwqgmvdgwygyhhsneqgsgyaadkestqkaidgv
tnkvnsiinkmntqfeavgrefnnlerrienlnkkmedgfldvwtynael
lvlmenertldfhdsnvknlydkvrlqlrdnakelgngcfefyhkcdnec
mesvkngtydypqyseearlnreeisgvklesmgtyqilaaa

[symbol]

[symbol]

[symbol]

[symbol]

[symbol]

### Sequence ID 3

DNA sequence of avian influenza haemagglutinin-7 (H7)

Agatctgacaaaatctgccttgggcatcatgccgtgtcaaacgggactaa
agtaaacacattaactgagagaggagtggaagtcgttaatgcaactgaaa
cggtggaacgaacaaacgttcccaggatctgctcaaaagggaaaaggaca
gttgacctcggtcaatgtggacttctggggacaatcactgggccacccc
aatgtgaccaattcctagaattttcggccgacttaattattgagaggcga
gaaggaagtgatgtctgttatcctgggaaattcgtgaatgaagaagctct
gaggcaaattctcagagagtcaggcggaattgacaaggagacaatgggat
tcacctacagcggaataagaactaatggagcaaccagtgcatgtaggaga
tcaggatcttcattctatgcagagatgaaatggctcctgtcaaacacaga
caatgctgctttcccgcaaatgactaagtcatacaagaacacaaggaaag
acccagctctgataatatgggggatccaccattccggatcaactacagaa
cagaccaagctatatgggagtggaaacaaactgataacagttgggagttc
taattaccaacagtcctttgtaccgagtccaggagcgagaccacaagtga
atggccaatctggaagaattgactttcattggctgatactaaaccctaat
gacacggtcactttcagtttcaatggggccttcatagctccagaccgtgc
aagctttctgagagggaagtccatgggaattcagagtgaagtacaggttg
atgccaattgtgaaggagattgctatcatagtggagggacaataataagt
aatttgccctttcagaacataaatagcaaggcagtaggaaaatgtccgag
atatgttaagcaagagagtctgctgttggcaacaggagtgaagaatgttc
ccgaaatcccaaagaggaggaggagaggcctatttggtgctatagcgggt
ttcattgaaaatggatgggaaggtttgattgatgggtggtatggcttcag
gcatcaaaatgcacaaggggagggaactgctgcagattacaaaagcaccc
aatcagcaattgatcaaataacagggaaattaaatcggcttatagaaaaa
actaaccaacagtttgagttaatagacaatgaattcactgaggttgaaaa
gcaaattggcaatgtgataaactggaccagagattccatgacagaagtgt
ggtcctataacgctgaactcttagtagcaatggagaatcagcacacaatt
gatctggccgactcagaaatgaacaaactgtacgaacgagtgaagagaca
actgagagagaatgccgaagaagatggcactggttgcttcgaaatatttc
acaagtgtgatgacgactgcatggccagtattagaaacaacacctatgat
cacagcaagtacagggaagaagcaatacaaaatagaatacagattgaccc
agtcaaactaagcagcggctacaaagatgtgatacttgcggccgc

### Sequence ID 4

Amino acid sequence of avian influenza haemagglutinin-7 (H7)

[symbol]

[symbol]

[symbol]

gsdkiclghhavsngtkvntltergvevvn
atetvertnvpricskgkrtvdlgqcgllgtitgppqcdqflefsadlii
erregsdvcypgkfvneealrqilresggidketmgftysgirtngatsa
crrsgssfyaemkwllsntdnaafpqmtksykntrkdpaliiwgihhsgs
tteqtklygsgnklitvgssnyqqsfvpspgarpqvngqsgridfhwlil
npndtvtfsfngafiapdrasflrgksmgiqsevqvdancegdcyhsggt
iisnlpfqnins [symbol] avgkcpryvkqeslllatg [symbol] knvpeipkrrrrglfga
iagfiengweglidgwygfrhqnaqgegtaadykstqsaidqitgklnrl
iektnqqfelidneftevekqignvinwtrdsmtevwsynaellvamenq
htidladsemnklyervkrqlrenaeedgtgcfeifhkcdddcmasirnn
tydhskyreeaiqnriqidpvklssgykdvilaaadykdhdgdykdhdid
ykdhdgaahhhhhh

### Sequence ID 5

DNA of Classical Swine Fever virus protein E2

GGTACCGGATCCATCAAGGTGCTGCGGGGCCAGGTGGTGCAGGGGGTGAT
CTGGCTGCTGCTGGTGACAGGCGCCCAGGGCCGGCTGGCCTGCAAAGAGG
ACCACAGATACGCCATCAGCACCACCAACGAGATCGGCCTGCTGGGCGCC
GAGGGCCTGACCACCACCTGGAAAGAGTACAACCACAACCTGCAGCTGGA
CGACGGCACCGTGAAGGCCATCTGCATGGCCGGCAGCTTCAAGGTGACCG
CCCTGAACGTGGTGTCCCGGCGCTACCTGGCCAGCCTGCACAAGGATGCC
CTGCCCACCTCCGTGACCTTCGAGCTGCTGTTCGACGGCACCAGCCCCCT
GACCGAGGAAATGGGCGACGACTTCGGCTTCGGCCTGTGCCCCTACGACA
CCAGCCCCGTGGTGAAGGGCAAGTACAACACCACCCTGCTGAACGGCAGC
GCCTTCTACCTGGTGTGCCCCATCGGCTGGACCGGCGTGATCGAGTGCAC
CGCCGTGAGCCCCACCACCCTGAGGACCGAGGTGGTGAAAACCTTCCGGC
GCGAGAAGCCCTTCCCCTACCGGCGGGACTGCGTGACCACCACAGTGGAG
AACGAGGACCTGTTCTACTGCAAGTGGGGCGGCAACTGGACCTGCGTGAA
GGGCGAGCCCGTGACCTACACCGGCGGACCCGTGAAGCAGTGCCGGTGGT
GCGGCTTCGACTTCAACGAGCCCGACGGCCTGCCCCACTACCCCATCGGC
AAGTGCATCCTGGCCAACGAGACCGGCTACCGGATCGTGGACAGCACCGA
CTGCAACCGGGACGGCGTGGTGATCAGCACCGAGGGCAGCCACGAGTGCC
TGATCGGCAACACCACAGTGAAGGTGCACGCCCTGGACGAGCGGCTGGGC
CCCATGCCCTGCCGGCCCAAAGAAATCGTGAGCAGCGCCGGACCCGTGCG
CAAGACCAGCTGCACCTTCAACTACGCCAAGACCCTGCGGAACCGGTACT
ACGAGCCCCGGGACAGCTACTTCCAGCAGTACATGCTGAAGGGCGAATAC
CAGTATTGGTTCGACCTGGACGTGACCGACCGGCACAGCGACTACTTCGC
CGAGTTTGCGGCCGCGAGCTC

### Sequence ID 6

Amino acid of Classical Swine Fever virus protein E2

GTGSIKVLRGQVVQGVIWLLLVTGAQGRLACKEDHRYAISTTNEIGLLGA
EGLTTTWKEYNHNLQLDDGTVKAICMAGSFKVTALNVVSRRYLASLHKDA
LPTSVTFELLFDGTSPLTEEMGDDFGFGLCPYDTSPVVKGKYNTTLLNGS
AFYLVCPIGWTGVIECTAVSPTTLRTEVVKTFRREKPFPYRRDCVTTTEN
EDLFYCKWGGNWTCVKGEPVTYTGGPVKQCRWCGFDFNE
PDGLPHYPIGKCILANETGYRIVDSTDCNRDGVVISTEGSHECLIGNTTV
KVHALDERLGPMPCRPKEIVSSAGPVRKTSCTFNYAKTLRNRYYEPRDSY
FQQYMLKGEYQYWFDLDVTDRHSDYFAEFAAAS

### Sequence ID 7

DNA and amino-acid sequence of Fasciola hepatica Cathepsin L3 (CL3 protein)

```
ggtaccggatccagcaacgacgtgagctggcacgagtggaagcggatgtacaacaaagag
  G T G S S N D V S W H E W K R M Y N K E
tacaacggcgccgacgaggaacaccggcggaacatctggggcaagaacgtgaagcacatc
  Y N G A D E E H R R N I W G K N V K H I
gaggaacacaacctgcggcacgaccggggcctggtgacctacaagctgggcctgaaccag
  E E H N L R H D R G L V T Y K L G L N Q
ttcaccgaccccaccttcgaggaattccaggccaagtacctgatggaaatgagccccgtg
  F T D P T F E E F Q A K Y L M E M S P V
agcgagagcctgagcgacggcgtgagctacgaggccgagggcaacgatgtgcccgccagc
  S E S L S D G V S Y E A E G N D V P A S
atcgactggcgggagtacggctacgtgaccgaggtgaaggaccagggccagtgcggcagc
  I D W R E Y G Y V T E V K D Q G Q C G S

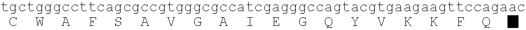

cagaccctgttcagcgagcagcagctggtcgactgcacccggcggttcggcaaccacggc
  Q T K F S E Q Q L V D C T R R F G N H G
tgtggcggcggatggatggaaaacgcctacaagtatctgaagaacagcggcctggaaacc
  C G G G W M E N A Y K Y L K N S G L E T
gccagctactacccctaccaggccgtggagtaccagtgccagtaccggaaagaactgggc
  A S Y Y P Y Q A V E Y Q C Q Y R K E L G
gtggccaaggtgaccggcgcctacaccgtgcacagcggcgacgagatgaagctgatgccc
  V A K V T G A Y T V H S G D E M K L M P
atggtgggccgggaaggccctgccgccgtggccgtggacgcccagagcgacttctacatg
  M V G R E G P A A V A V D A Q S D F Y M
tacgagagcggcatctttcagagccagacctgcaccagcagaagcgtgacccagcgggtc
  Y E S G I F Q S Q T C T S R S V T H A V
ctggccgtgggctacggcaccgagtccggcaccgactactggattctgaagaactcctgg
  L A V G Y G T E S G T D Y W I L K N S W
ggcaagtggtggggcgaggacggctacatgcggttcgcccggaaccggggcaacatgtgc
  G K W W G E D G Y M R F A R N R G N M C
gccatcgccagcgtggcctccgtgcctatggtggagcggttccccgcggccgcgagctc
  A I A S V A S V P M V E R F P A A A S
```

### Sequence ID 8

Amino acid sequence of Fasciola hepatica Cathepsin L3 peptide with an amino-terminal Cys extension (CL3 peptide)

CSNDVSWHEWKRMYNKEYNG

1. A method for producing composition comprising at least one peptide, polypeptide, protein, glycoprotein and/or lipoprotein, said method comprising:
providing said composition with at least one cross-β structure.

2. The method according to claim 1, wherein said cross-β structure is induced in at least part of said composition.

3. A method of preparing a vaccine for the prophylaxis of an infectious disease, the improvement comprising:
using cross-β structures in the preparation of the vaccine.

4. A method of preparing a composition that induces an immune response against an infectious agent, the improvement comprising:
using cross-β structures induced in a protein component of the infectious agent in the preparation of the composition.

5. The method according to claim 4, wherein said protein component is a viral protein and wherein said infectious agent is a virus.

6. The method according to claim 4, wherein said protein component is a bacterial protein and wherein said infectious agent is a bacterium.

7. A subunit vaccine comprising at least one viral protein, wherein at least 4-50% of said viral protein is in a conformation comprising cross-β structures.

8. A subunit vaccine comprising at least one bacterial protein, wherein at least 4-50% of said bacterial protein is in a conformation comprising cross-β structures.

9. The subunit vaccine of claim 7, comprising at least two viral proteins.

10. The subunit vaccine of claim 8, comprising at least two bacterial proteins.

11. A method for improving immunogenicity of a composition comprising at least one peptide, polypeptide, protein, glycoprotein and/or lipoprotein, said method comprising
contacting at least one of said peptide, polypeptide, protein, glycoprotein and/or lipoprotein with a cross-β inducing agent, thereby providing said composition with additional cross-β structures and improving the composition's immunogenicity.

12. A method for enhancing immunogenicity of a vaccine composition comprising at least one peptide, polypeptide, protein, glycoprotein and/or lipoprotein, said method comprising:
contacting at least one of said peptide, polypeptide, protein, glycoprotein and/or lipoprotein with a cross-β inducing agent, thereby providing said vaccine composition with additional cross-β structures and enhancing the vaccine composition's immunogenicity.

13. A method for determining the amount of cross-β structures in a vaccine composition, said method comprising
contacting said vaccine composition with at least one cross-β structure binding compound and relating the amount of bound cross-β structures to the amount of cross-β structures present in the vaccine composition.

14. A method of preparing a composition for the prophylaxis and/or treatment of cancer, the method comprising:
using cross-β structures in the preparation of the composition.

15. A method of preparing an immunogenic composition, the method comprising:
using cross-β structures in the preparation of an immunogenic composition for:
immuno-castration, and/or
the prophylaxis and/or treatment of atherosclerosis, amyloidoses, autoimmune diseases, graft-versus-host rejections and/or transplant rejections.

16. A composition comprising a bacterial or parasitic or viral antigen, said antigen comprising at least between 4-50% of said antigen is in a cross-β structure conformation.

17. The composition of claim 16 wherein said antigen comprises HPV E6 protein, HPV E7 protein, Influenza haemaglutinin H5, Influenza haemaglutinin H7, pestivirus E2 protein, Fasciola hepatica CL3 protein and/or Neisseria PorA protein.

18. (canceled)

19. The method according to claim 1, wherein said at least one peptide, polypeptide, protein, glycoprotein and/or lipoprotein, comprises HPV E6, HPV E7, Fasciola hepatica CL3, Influenza H5, Influenza H7, pestivirus E2 protein and/or Neisseria PorA protein.

20. A composition comprising a β2glycoprotein I or an antigenic peptide thereof, said immunogenic composition comprising at least between 4-67% of said β2glycoprotein I or antigenic peptide thereof in a cross-β structure conformation.

21. The composition of claim 20, wherein said β2glycoprotein I, or antigenic peptide thereof, is coupled to or mixed with another peptide of which at least between 4-67% of said peptide is in a cross-β structure conformation.

22. A method of treating or preventing an autoimmune disease in a subject, said method comprising:
administering, to the subject, the composition of claim 20 for the prophylaxis or treatment of an autoimmune disease.

23. A composition comprising a bacterial or parasitic or viral antigenic peptide wherein said antigenic peptide comprises at least between 4-67% of said antigenic peptide in a cross-β structure conformation.

24. The composition of claim 23, further comprising another peptide comprising at least between 4-67% of said other peptide in a cross-β structure conformation.

25. The composition according to claim 24, wherein said another protein comprises OVA or KLH or a combination of both.

26. The composition of claim 24, further comprising an adjuvant.
